(19) United States
(12) Patent Application Publication
(10) Pub. No.: US 2019/0135923 A1
(43) Pub. Date: May 9, 2019

(54) COMPOSITIONS AND TREATMENT METHODS FOR CANCER IMMUNOTHERAPY

(71) Applicant: Biograph 55, Inc., South San Francisco, CA (US)

(72) Inventors: Paul C. TUMEH, San Francisco, CA (US); Peter THOMPSON, Bellevue, WA (US)

(21) Appl. No.: 16/242,333

(22) Filed: Jan. 8, 2019

Related U.S. Application Data
(63) Continuation of application No. PCT/US2017/041256, filed on Jul. 7, 2017.
(60) Provisional application No. 62/360,280, filed on Jul. 8, 2016.
(60) Provisional application No. 62/360,244, filed on Jul. 8, 2016.
(60) Provisional application No. 62/423,741, filed on Nov. 17, 2016.
(60) Provisional application No. 62/435,041, filed on Dec. 15, 2016.
(60) Provisional application No. 62/490,517, filed on Apr. 26, 2017.

(51) Int. Cl. C07K 16/28; A61P 35/00

(52) U.S. Cl. CPC C07K 16/2827; C07K 16/2818; A61P 35/00

(57) ABSTRACT

Provided herein are analytical methods to be applied to samples, diagnostic methods, methods of determining a therapeutic regimens, and methods of treating cancer in a subject comprising compositions described herein.

### CROSS REFERENCE

This application claims priority to U.S. Application No. 62/360,244, filed Jul. 8, 2016, U.S. Application No. 62/360,280, filed Jul. 8, 2016, U.S. Application No. 62/423,741, filed Nov. 17, 2016, U.S. Application No. 62/435,041, filed Dec. 15, 2016, and U.S. Application No. 62/490,517, filed Apr. 26, 2017, which are entirely incorporated herein by reference.

### BACKGROUND

Cancer is a leading cause of death throughout the world. One approach to cancer treatment is cancer immunotherapy. Cancer immunotherapy involves the use of compositions and methods to elicit or enhance an individual's immune system against cancerous cells.

### BRIEF SUMMARY

In an aspect, the present disclosure provides a method of treating a subject in need thereof, comprising: administering a therapeutically effective amount of at least one B-cell activating factor (BAFF) inhibitor and/or at least one B-cell activating factor receptor (BAFF-R) inhibitor, or a salt thereof; and administering a therapeutically effective amount of at least one checkpoint inhibitor or a salt thereof. In some embodiments, the subject has been diagnosed with a cancer or a tumor. In some embodiments, the method is a method of treating a cancer or a tumor in the subject. In some embodiments, the subject has not previously undergone treatment for the cancer or the tumor. In some embodiments, the subject has previously or is currently undergoing treatment for the cancer or the tumor. In some embodiments, the subject has previously undergone treatment with surgery or radiation, and the subject remains in remission. In some embodiments, the subject has previously undergone treatment with surgery, radiation, an anticancer agent, and any combination thereof, and the cancer or the tumor was at least partially refractive to the treatment.

In some embodiments, the subject was previously treated with surgery, radiation therapy, an anticancer agent, and any combination thereof, and the cancer or the tumor re-occurred in the subject following a period of remission, wherein the period of remission was at least about: 1 month, 2 months, 3 months, 6 months, 1 year, 2 years, 3 years, 4 years, 5 years, 6 years, 7 years, 8 years, 9 years, or 10 years. In some embodiments, the subject was previously treated with a checkpoint inhibitor or a salt thereof, and the cancer or the tumor was at least partially refractive to the checkpoint inhibitor or a salt thereof. In some embodiments, the subject was previously treated with a checkpoint inhibitor or a salt thereof, and the cancer or the tumor re-occurred in the subject following a period of remission, wherein a period of the remission was at least about: 1 month, 2 months, 3 months, 6 months, 1 year, 2 years, 3 years, 4 years, 5 years, 6 years, 7 years, 8 years, 9 years, or 10 years. In some embodiments, the checkpoint inhibitor or a salt thereof is administered sequentially with the BAFF inhibitor and/or BAFF-R inhibitor or a salt thereof.

In some embodiments, the checkpoint inhibitor or a salt thereof is administered concurrently with the BAFF inhibitor and/or BAFF-R inhibitor or a salt thereof. In some embodiments, the checkpoint inhibitor is selected from the group consisting of an agent that binds to anti-programmed cell death protein 1 (PD-1), anti-programmed death ligand 1 (PD-L1), anti-programmed death ligand 2 (PD-L2), cytotoxic T-lymphocyte-associated protein 4 (CTLA4), cluster of differentiation 276 (B7-H3), V-set domain-containing T-cell activation inhibitor 1 (B7-H4), T-cell immunoglobulin and mucin-domain containing-3 (TIM-3), lymphocyte-activation gene 3 (LAG3), Indoleamine-pyrrole 2,3-dioxygenase (IDO), a salt of any one thereof, and any combination thereof. In some embodiments, the checkpoint inhibitor or a salt thereof is a PD-1 inhibitor or a salt thereof.

In some embodiments, the PD-1 inhibitor or a salt thereof is selected from the group consisting of nivolumab, pembrolizumab, pidilizumab, BGB-A31, MEDI0680 (AMP-514), and a salt of any one thereof. In some embodiments, the PD-1 inhibitor is nivolumab or a salt thereof. In some embodiments, the PD-1 inhibitor is pembrolizumab or a salt thereof.

In some embodiments, the PD-1 inhibitor is pidilizumab or a salt thereof. In some embodiments, the PD-1 inhibitor is BGB-A31 or a salt thereof. In some embodiments, the PD-1 inhibitor is MEDI0680 (AMP-514) or a salt thereof. In some embodiments, the checkpoint inhibitor or a salt thereof is a PD-L1 inhibitor or a salt thereof. In some embodiments, the PD-L1 inhibitor or a salt thereof is selected from the group consisting of atezolizumab, avelumab, durvalumab, MDX-1105, MSB0010718C, and a salt of any one thereof. In some embodiments, the PD-L1 inhibitor is atezolizumab or a salt thereof. In some embodiments, the PD-L1 inhibitor is avelumab or a salt thereof. In some embodiments, the PD-L1 inhibitor is durvalumab or a salt thereof.

In some embodiments, the PD-L1 inhibitor is MDX-1105 or a salt thereof. In some embodiments, the PD-L1 inhibitor is MSB0010718C or a salt thereof. In some embodiments, the checkpoint inhibitor is a CTLA4 inhibitor or a salt thereof. In some embodiments, the CTLA4 inhibitor or a salt thereof is selected from the group consisting of ipilimumab, tremelimumab, AGEN1884, and a salt of any one thereof. In some embodiments, the CTLA4 inhibitor is ipilimumab or a salt thereof. In some embodiments, the CTLA4 inhibitor is tremelimumab or a salt thereof. In some embodiments, the CTLA4 inhibitor is AGEN1884 or a salt thereof.

In some embodiments, the checkpoint inhibitor or a salt thereof is a LAG3 inhibitor or a salt thereof. In some embodiments, the LAG3 inhibitor is BMS-986016 or a salt thereof. In some embodiments, the checkpoint inhibitor or a salt thereof is a TIM3 inhibitor or a salt thereof. In some embodiments, the TIM3 inhibitor or a salt thereof is selected from the group consisting of MBG453, TSR-022, and a salt of any one thereof. In some embodiments, the TIM3 inhibitor is MBG453 or a salt thereof. In some embodiments, the TIM3 inhibitor is TSR-022 or a salt thereof.

In an aspect, the present disclosure provides a method of treating a subject in need thereof, comprising: administering a therapeutically effective amount of at least one BAFF and/or BAFF-R inhibitor or a salt thereof; and administering at least one immune agonist agent or a salt thereof. In some embodiments, the immune agonist agent or a salt thereof is selected from the group consisting of an agent that binds to glucocorticoid-induced tumor necrosis factor receptor-related protein (GITR), cluster of differentiation 134 (OX40), cluster of differentiation 137 (CD137), cluster of differentiation 40 (CD40), receptor (TLR), or a salt of any one thereof, and any combination thereof. In some embodiments, the immune agonist agent is a GITR agonist or a salt thereof.

In some embodiments, the GITR agonist or a salt thereof is selected from the group consisting of an agent that binds to TRX518, GWN323, MEDI1873, INCAGN01876, and a salt of any one thereof. In some embodiments, the GITR agonist is TRX518 or a salt thereof. In some embodiments, the GITR agonist is GWN323 or a salt thereof. In some embodiments, the GITR agonist is MEDI1873 or a salt thereof. In some embodiments, the GITR agonist is INCAGN01876 or a salt thereof. In some embodiments, the immune agonist agent is an OX40 agonist or a salt thereof.

In some embodiments, the OX40 agonist is selected from the group consisting of GSK3174998, PF-04518600, MEDI6469, INCAGN01949, and a salt of any one thereof. In some embodiments, the OX40 agonist is GSK3174998 or a salt thereof. In some embodiments, the OX40 agonist is PF-04518600 or a salt thereof. In some embodiments, the OX40 agonist is MEDI6469 or a salt thereof. In some embodiments, the OX40 agonist is INCAGN01949 or a salt thereof.

In some embodiments, the immune agonist agent is a cluster of differentiation 137 (4-1BB) agonist or a salt thereof. In some embodiments, the 4-1BB agonist is urelumab, utomilumab, or a salt of any one thereof. In some embodiments, the 4-1BB agonist is isurelumab or a salt thereof. In some embodiments, the 4-1BB agonist is isutomilumab or a salt thereof. In some embodiments, the immune agonist agent is a CD40 agonist or a salt thereof. In some embodiments, the CD40 agonist is APX005M, CP870893, or a salt thereof. In some embodiments, the CD40 is APX005M or a salt thereof. In some embodiments, the CD40 is CP870893 or a salt thereof. In some embodiments, the immune agonist agent is a TLR agonist or a salt thereof. In some embodiments, the TLR agonist is selected from the group consisting of an agent that hinds to TLR-1, TLR-2, TLR-3, TLR-4, TLR-5, TLR-6, TLR-7, TLR-8, TLR-9, TLR-13, a salt or any one thereof, and any combination thereof.

In an aspect, the present disclosure provides a method of treating a subject in need thereof, comprising: administering a therapeutically effective amount of at least one BAFF and/or BAFF-R inhibitor or a salt thereof; and administering at least one vaccine agent or a salt thereof. In some embodiments, the vaccine agent is selected from the group consisting of MAGE-3, NY-ESO-1, TRAG-3, p53, at least one or more α-actinin-4 and malic enzymes, carcinoembryonic antigen, HER2, MUC1, survivin, WT-1, PRAME, Survivin-2b, Bacillus Calmette-Guerin, MVAX, at least one or more heat shock proteins, keyhole limpet hemocyanin, interleukin-2, QS21, montanide ISA-51, granulocyte monocyte-colony stimulating factor, GVAX, GI-4000, CDX-1307, IMA910, TroVAX, CRS-207, CA-9, a salt of any one thereof, and any combination thereof.

In an aspect, the present disclosure provides a method of treating a subject in need thereof, comprising: administering a therapeutically effective amount of at least one BAFF and/or BAFF-R inhibitor or a salt thereof; and administering at least one oncolytic viral based agent or a salt thereof. In some embodiments, the oncolytic viral based agent is selected from the group consisting of enadenotucirev, talimogene laherparepvec, reolysin, CG0070, Pexastimogene devacirepvec, cavatak, oncolytic vesicular stomatitis virus, ONCOS-102, a salt of any one thereof, and any combination thereof.

In an aspect, the present disclosure provides a method of treating a subject in need thereof, comprising: administering a therapeutically effective amount of at least one BAFF and/or BAFF-R inhibitor or a salt thereof; and administering at least one cell-based therapy, any cell derivative thereof, or a salt of any one thereof. In some embodiments, the cell-based therapy is selected from the group consisting of: at least one or more autologous lymphocytes, at least one or more genetically engineered autologous lymphocytes, at least one or more chimeric antigen receptor cells, at least one or more chimeric antigen receptor T-cells, at least one or more dendritic cell based vaccines, a salt of any one thereof, and any combination thereof.

In an aspect, the present disclosure provides a method of treating a subject in need thereof, comprising: administering a therapeutically effective amount of at least one BAFF and/or BAFF-R inhibitor or a salt thereof; and administering at least one chemotherapeutic agent or a salt thereof. In some embodiments, the chemotherapeutic agent is an alkylating agent, an antimetabolite agent, a plant alkaloid agent, an antitumor antibiotic, a salt of any one thereof, and any combination thereof.

In an aspect, the present disclosure provides a method of treating a subject in need thereof, comprising: administering a therapeutically effective amount of at least one BAFF and/or BAFF-R inhibitor or a salt thereof; and administering at least one targeted therapeutic agent or a salt thereof. In some embodiments, the targeted therapeutic agent is an agent that inhibits signal transduction, angiogenesis, hormone expression, or any combination thereof. In some embodiments, the BAFF inhibitor and/or BAFF-R inhibitor prevents binding of BAFF or a BAFF-induced ligand to a BAFF-R, is a partial antagonist of a BAFF, is a partial agonist of a BAFF, is a competitive antagonist of a BAFF-R, is a non-competitive antagonist of a BAFF-R receptor, or any combination thereof.

In some embodiments, the BAFF inhibitor is an antibody, antigen binding fragment, a bispecific antibody, or a recombinant fusion protein. In some embodiments, the BAFF inhibitor antibody is human, humanized, chimeric, composite, polyclonal or monoclonal. In some embodiments, the BAFF antibody or antigen binding fragment blocks interaction of molecules to BAFF. In some embodiments, the BAFF inhibitor is selected from the group consisting of tabalumab, BAFF/IL-17 bispecific antibody, ardenermin, briobacept, blisibimod, belimumab, a transmembrane activator and calcium-modulator and cyclophilin ligand interactorcept, a salt of any one thereof, and any combination thereof.

In some embodiments, the BAFF inhibitor is selected from the group consisting of MEDI-700, NOV-5, rGel/BLyS, a salt of any one thereof, and any combination thereof. In some embodiments, the BAFF inhibitor is tabalumab or a salt thereof. In some embodiments, the BAFF inhibitor is blisibimod or a salt thereof. In some embodiments, the BAFF inhibitor is belimumab or a salt thereof.

In an aspect, the present disclosure provides a method of treating a subject in need thereof, comprising: administering a therapeutically effective amount of an immunotherapeutic agent or a salt thereof, wherein the immunotherapeutic agent or a salt thereof inhibits a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof; wherein the immunotherapeutic agent is co-administered with a checkpoint inhibitor. In some embodiments, the subject has been diagnosed with a cancer or a tumor. In some embodiments, the method is a method of treating a cancer or a tumor in the subject. In some embodiments, the subject has previously or is currently undergoing treatment for the cancer or the tumor. In some embodiments, the subject was previously treated with a checkpoint inhibitor, and the cancer or the tumor was at least partially refractive to the checkpoint inhibitor.

In some embodiments, the method further comprises administering a therapeutically effective amount of the checkpoint inhibitor. In some embodiments, the checkpoint inhibitor is a salt. In some embodiments, the checkpoint inhibitor is selected from the group consisting of an anti-programmed cell death protein 1 agent, an anti-programmed death ligand 1 agent, an anti-programmed death ligand 2 agent, a salt of any of these, and any combination thereof. In some embodiments, the method further comprises administering the anti-programmed cell death protein 1 agent or a salt thereof. In some embodiments, the anti-programmed cell death protein 1 agent or a salt thereof is selected from the group consisting of vemurafenib, erlotinib, docetaxel, nivolumab, pembrolizumab, and a salt of any of these.

The method of any one of the preceding claims, wherein the anti-programmed cell death protein 1 agent or a salt thereof is vemurafenib. In some embodiments, the anti-programmed cell death protein 1 agent or a salt thereof is erlotinib. In some embodiments, the anti-programmed cell death protein 1 agent or a salt thereof is pembrolizumab. In some embodiments, the method comprises administering the anti-programmed death ligand 1 agent or a salt thereof. In some embodiments, the anti-programmed death ligand 1 agent or a salt thereof is selected from the group consisting of atezolizumab, avelumab, durvalumab, and a salt of any of these.

In some embodiments, the immunotherapeutic agent or a salt thereof at least partially prevents binding of a ligand to a B-cell activating factor receptor, is a partial antagonist of a B-cell activating factor receptor, is a partial agonist of a B-cell activating factor receptor, is a competitive antagonist of a B-cell activating factor receptor, is a non-competitive antagonist of a B-cell activating factor receptor, or any combination thereof. In some embodiments, the immunotherapeutic agent or a salt thereof at least partially prevents ligand binding to a transmembrane activator and calcium-modulator and cyclophilin ligand interactor receptor, is a partial antagonist of a transmembrane activator and calcium-modulator and cyclophilin ligand interactor receptor, is a partial agonist of a transmembrane activator and calcium-modulator and cyclophilin ligand interactor receptor, or any combination thereof.

In some embodiments, the immunotherapeutic agent or a salt thereof at least partially prevents ligand binding to a B-cell maturation antigen receptor, is a partial antagonist of a B-cell maturation antigen receptor, is a partial agonist of a B-cell maturation antigen receptor, or any combination thereof. In some embodiments, the immunotherapeutic agent is an antibody or a salt thereof. In some embodiments, the immunotherapeutic agent is a human or humanized monoclonal antibody or a salt thereof. In some embodiments, the immunotherapeutic agent or a salt thereof is selected from the group consisting of ardenermin, briobacept, blisibimod, belimumab, atransmembrane activator and calcium-modulator and cyclophilin ligand interactorcept, tabalumab, a salt of any of these, and any combination thereof. In some embodiments, the immunotherapeutic agent or a salt thereof is selected from the group consisting of MEDI-700, NOV-5, rGel/BLyS, a salt of any of these, and any combination thereof.

In some embodiments, the immunotherapeutic agent is tabalumab or a salt thereof. In some embodiments, the immunotherapeutic agent is blisibimod or a salt thereof. In some embodiments, the immunotherapeutic agent is belimumab or a salt thereof. In some embodiments, the method further comprises, before the administering: determining a count of cluster of differentiation 8 (CD8) protein, and at least one selected from the group consisting of: cluster of differentiation 19 (CD19) protein, cluster of differentiation 20 protein (CD20), cluster of differentiation 138 (CD138) protein, B-cell activating factor protein, B-cell activating factor receptor protein, and paired box Pax-5 (PAX5) protein, in a cancer or a tumor or a cancer sample or a tumor sample of a subject. In some embodiments, the at least one of: CD19, CD20, CD138, B-cell activating factor, B-cell activating factor-R, and PAX5 is present in the cancer or the tumor or the cancer sample or the tumor sample of the subject. In some embodiments, the at least one of: CD19, CD20, CD138, B-cell activating factor. B-cell activating factor-R, and PAX5 is individually present in the cancer or the tumor or the cancer sample or the tumor sample of the subject in an amount ranging from 1 protein to at least about 10,000,000 proteins. In some embodiments, the method further comprises monitoring the treatment after the administering.

In some embodiments, the treatment maintains at least partial remission of the cancer or the tumor, wherein a period of the remission was at least about: 1 month, 2 months, 3 months, 6 months, 1 year, 2 years, 3 years, 4 years, 5 years, 6 years, 7 years, 8 years, 9 years, or 10 years. In some embodiments, the treatment comprises, after administering, delaying progression of the cancer or the tumor in the subject. In some embodiments, the treatment comprises, after administering, regression of the cancer or the tumor. In some embodiments, regression is a reduction in mass of the cancer or the tumor, a reduction in volume of the cancer or the tumor, or both. In some embodiments, the treatment comprises prolonging the subject's life. In some embodiments, the subject has previously shown at least partial refraction to a monotherapy for the cancer or the tumor. In some embodiments, the subject has shown refraction to a monotherapy for the cancer or the tumor. In some embodiments, the cancer has metastasized from a first location of the subject to a second location of the subject.

In some embodiments, the cancer at the first location of the body is less than fully responsive to the monotherapy, and wherein the cancer at the second location of the body is responsive to the treatment. In some embodiments, the checkpoint inhibitor and the immunotherapeutic agent are synergistic. In some embodiments, the synergy on the cancer or the tumor is at least 10% more than an additive effect. In some embodiments, a heatmap of a cancer sample or a tumor sample of the subject shows a profile substantially similar to that of FIG. 8.

In some embodiments, the anti-programmed cell death protein 1 agent, the anti-programmed death ligand 1 agent, the anti-programmed death ligand 2 agent, or a salt thereof or any combination thereof, and the immunotherapeutic agent or a salt thereof are administered concurrently. In some embodiments, the anti-programmed cell death protein 1 agent, the anti-programmed death ligand 1 agent, the anti-programmed death ligand 2 agent, or a salt thereof or any combination thereof, and the immunotherapeutic agent or a salt thereof are administered sequentially. In some embodiments, the anti-programmed cell death protein 1 agent, the anti-programmed death ligand 1 agent, the anti-programmed death ligand 2 agent, or a salt thereof or any combination thereof, and the immunotherapeutic agent or a salt thereof are administered in different formulations within a same treatment schedule.

In some embodiments, the anti-programmed cell death protein 1 agent, the anti-programmed death ligand 1 agent, the anti-programmed death ligand 2 agent, or a salt thereof or any combination thereof, is administered at least once a week. In some embodiments, the anti-programmed cell death protein 1 agent, the anti-programmed death ligand 1 agent, the anti-programmed death ligand 2 agent, or a salt thereof or any combination thereof, is administered at least once a day. In some embodiments, the anti-programmed cell death protein 1 agent, the anti-programmed death ligand 1 agent, the anti-programmed death ligand 2 agent, or a salt thereof or any combination thereof, is administered at least once during a treatment schedule.

In some embodiments, the immunotherapeutic agent or a salt thereof is administered at least once a week. In some embodiments, the immunotherapeutic agent or a salt thereof is administered at least once a day. In some embodiments, the immunotherapeutic agent or a salt thereof is administered at least once during a treatment schedule. In some embodiments, the immunotherapeutic agent or a salt thereof is administered in an amount from about 0.1 mg to about 1,000 mg per kg body weight.

In some embodiments, the immunotherapeutic agent or a salt thereof is administered in an amount from about 0.1 mg to about 100 mg per kg body weight. In some embodiments, the immunotherapeutic agent or a salt thereof is administered in an amount from about 1 mg to about 50 mg per kg body weight. In some embodiments, the immunotherapeutic agent or a salt thereof is administered in a pharmaceutical composition. In some embodiments, the anti-programmed cell death protein 1 agent, the anti-programmed death ligand 1 agent, the anti-programmed death ligand 2 agent, or a salt thereof or any combination thereof, is administered in an amount from about 0.1 mg to about 1,000 mg per kg body weight. In some embodiments, the anti-programmed cell death protein 1 agent, the anti-programmed death ligand 1 agent, the anti-programmed death ligand 2 agent, or a salt thereof or any combination thereof, is administered in an amount from about 1 mg to about 100 mg per kg body weight. In some embodiments, the anti-programmed cell death protein 1 agent, the anti-programmed death ligand 1 agent, the anti-programmed death ligand 2 agent, or a salt of any one thereof, or any combination thereof, are individually present as a pharmaceutical formulation.

In some embodiments, the pharmaceutical formulation is in unit dose form. In some embodiments, the pharmaceutical formulation further comprises a pharmaceutically-acceptable excipient. In some embodiments, at least one of the anti-programmed cell death protein 1 agent, the anti-programmed death ligand 1 agent, the anti-programmed death ligand 2 agent, or a salt of any one thereof, or any combination thereof; and the immunotherapeutic agent or salt thereof is isolated and purified. In some embodiments, the administration is oral. In some embodiments, the administration is topical, intravenous, intramuscular, or spinal. In some embodiments, the administration is administered directly to the cancer or the tumor. In some embodiments, the administration is an administration at a location different than the cancer or the tumor. In some embodiments, the subject has been previously diagnosed with the cancer or the tumor. In some embodiments, the cancer or the tumor is a solid tumor or a solid cancer. In some embodiments, the cancer or the tumor is a liquid cancer or a liquid tumor. In some embodiments, the cancer or the tumor is malignant. In some embodiments, the cancer or the tumor is present in an organ. In some embodiments, the tumor or the cancer is present in at least one of: blood, lymph, cerebral spinal fluid. In some embodiments, the cancer or the tumor is located in a head or neck region, abdominal region, a upper limb, a lower limb, skin, blood, digestive tract, germ cell, or nervous system. In some embodiments, the method comprises treating the cancer, wherein the cancer is prostate cancer, testicular cancer, breast cancer, brain cancer, pancreatic cancer, colon cancer, thyroid cancer, stomach cancer, lung cancer, melanoma, multiple myeloma, Hodgkin's lymphoma, or ovarian cancer.

In an aspect, the present disclosure provides a composition comprising an anti-programmed cell death protein 1 agent, an anti-programmed death ligand 1 agent, an anti-programmed death ligand 2 agent, or a salt of any one thereof, or any combination thereof, and an immunotherapeutic agent or a salt thereof. In some embodiments, the composition is a pharmaceutical composition. In some embodiments, the pharmaceutical composition is in unit dose form. In some embodiments, the immunotherapeutic agent or a salt thereof is an antibody or a salt thereof. In some embodiments, the immunotherapeutic agent or a salt thereof is a human or humanized monoclonal antibody or a salt thereof. In some embodiments, the immunotherapeutic agent or a salt thereof is selected from the group consisting of ardenermin, briobacept, blisibimod, belimumab, atransmembrane activator and calcium-modulator and cyclophilin ligand interactorcept, MEDI-0700, NOV-5, rGel/BLyS, tabalumab, and a salt of any of these. In some embodiments, the immunotherapeutic agent or a salt thereof is tabalumab or a salt thereof. In some embodiments, the method further comprises a pharmaceutically acceptable excipient.

In some embodiments, the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof are independently administered in an amount from about 0.1 mg to about 10 g. In some embodiments, the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof are independently 0.001% to 99% by weight of the composition. In some embodiments, the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof are independently administered in an amount of about 0.1 mg to about 100 mg per kg body weight. In some embodiments, the composition is in a form of a tablet, a capsule, a gel, or a liquid formulation.

In an aspect, the present disclosure provides a kit comprising a composition described herein. In some embodiments, the kit contains instructions for use.

In an aspect, the present disclosure provides a method of making a kit described herein, comprising contacting or combining the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof. In some embodiments, the composition is in a form of a tablet, a capsule, a gel, or a liquid formulation. In some embodiments, the method comprises contacting or combining the anti-programmed cell death protein 1 agent, the anti-programmed death ligand 1 agent, the anti-programmed death ligand 2 agent, or a salt thereof or any combination thereof, and the immunotherapeutic agent or a salt thereof.

In an aspect, the present disclosure provides a method of selecting a therapeutic regimen, comprising: determining the presence or absence of cluster of differentiation 8 (CD8) protein, and at least one of: B-cell activating factor (BAFF) protein, B-cell activating factor receptor (BAFF-R) protein, and paired box Pax-5 (PAX5) protein in a cancer or a tumor of a subject or a cancer sample or a tumor sample of the subject; if CD8 protein and at least one of B-cell activating factor, B-cell activating factor-R, and PAX5 are present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof; if CD8 protein and at least one of B-cell activating factor, B-cell activating factor-R, and PAX5 is not present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof. In some embodiments, the method further comprises determining whether cluster of differentiation 19 (CD19) protein, cluster of differentiation 20 (CD20) protein, cluster of differentiation 138 (CD138) protein, or any combination thereof is present. In some embodiments, the method further comprises treating the subject. In some embodiments, the treating comprises: administering a therapeutically effective amount of an immunotherapeutic agent or a salt thereof, wherein the immunotherapeutic agent or a salt thereof inhibits a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof; wherein the immunotherapeutic agent is co-administered with a checkpoint inhibitor.

In some embodiments, the subject has been diagnosed with a cancer or a tumor. In some embodiments, the subject has previously or is currently undergoing treatment for the cancer or the tumor. In some embodiments, the method comprises administering a therapeutically effective amount of the checkpoint inhibitor. In some embodiments, the checkpoint inhibitor is selected from the group consisting of an anti-programmed cell death protein 1 agent, an anti-programmed death ligand 1 agent, an anti-programmed death ligand 2 agent, a salt of any of these, and any combination thereof. In some embodiments, the method comprises administering the anti-programmed cell death protein 1 agent of a salt thereof.

In some embodiments, the anti-programmed cell death protein 1 agent or a salt thereof is selected from the group consisting of vemurafenib, erlotinib, docetaxel, nivolumab, pembrolizumab, and a salt of any of these. In some embodiments, the anti-programmed cell death protein 1 agent or a salt thereof is vemurafenib. In some embodiments, the anti-programmed cell death protein 1 agent or a salt thereof is erlotinib. In some embodiments, the anti-programmed cell death protein 1 agent or a salt thereof is pembrolizumab. In some embodiments, the immunotherapeutic agent is tabalumab or a salt thereof. In some embodiments, the immunotherapeutic agent is belimumab or a salt thereof.

In some embodiments, the cancer or the tumor is prostate cancer, testicular cancer, breast cancer, brain cancer, pancreatic cancer, colon cancer, thyroid cancer, stomach cancer, lung cancer, or ovarian cancer. In some embodiments, the cancer or the tumor contacts a blood vessel. In some embodiments, the cancer or the tumor is in the interior of a blood vessel.

In an aspect, the present disclosure provides a method of selecting a therapeutic regimen, comprising: determining a count of B cells, T cells, and plasma cells in a cancer or a tumor of a subject or a cancer sample or a tumor sample of the subject; if a count of T cells in the cancer or the tumor or the cancer sample or the tumor sample is greater than 500 cells/mm2 and a count of B cells is less than <150 cells/mm2, selecting a therapeutic regimen comprising an anti-programmed cell death protein 1 agent, an anti-programmed death ligand 1 agent, an anti-programmed death ligand 2 agent, or a salt thereof or any combination thereof; if a count of T cells in the cancer or the tumor or the cancer sample or the tumor sample is greater than 300 cells/mm2 and a count of B cells, a count of plasma cells, or a combination of B cells and plasma cells is greater than 100 cells/mm2, selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof. In some embodiments, the method further comprises conveying a result via a communication medium.

In an aspect, the present disclosure provides a method of treating or maintaining remission of a cancer or a tumor in a subject, comprising: administering an anti-programmed cell death protein 1 agent, an anti-programmed death ligand 1 agent, an anti-programmed death ligand 2 agent, or a salt thereof or any combination thereof, if a percentage of T cells in a cancer sample or a tumor sample of a subject is greater than 10% and a percentage of B cells is less than 5%, or administering an anti-programmed cell death protein 1 agent, an anti-programmed death ligand 1 agent, an anti-programmed death ligand 2 agent, or a salt thereof or any combination thereof, and an immunotherapeutic agent or a salt thereof if a percentage of T cells in a cancer or a tumor sample of the subject is less than 10% and a percentage of B cells, a percentage of plasma cells, or a combination of the percentage of B cells and the percentage of plasma cells is greater than 10% in the cancer sample of the subject. In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following is observed when compared to a count prior to treatment: a percentage of T cells, a percentage of B cells, and a percentage of plasma cells. In some embodiments, the cancer or the tumor is prostate cancer, testicular cancer, breast cancer, brain cancer, pancreatic cancer, colon cancer, thyroid cancer, stomach cancer, lung cancer, or ovarian cancer.

In an aspect, the present disclosure provides a method of assessing a likelihood of a subject having a cancer or a tumor exhibiting a clinically beneficial response to treatment, the method comprising: assessing a count of T cells, B cells, and plasma cells in a cancer or a tumor sample of a subject; and calculating, using a computer system, a probability of treatment responsiveness based on (1) a ratio between the count of T cells and a reference count of T cells, (2) a ratio between the count of B cells and a reference count of B cells, and/or (3) a ratio between the count of plasma cells and a reference count of plasma cells. In some embodiments, the method further comprises designating the subject as having a high probability of exhibiting a clinically beneficial response to treatment if the ratio between the count of B cells and the reference count of B cells is less than 1. In some embodiments, the method further comprises designating the subject as having a high probability of exhibiting a clinically beneficial response to treatment if the ratio between the count of plasma cells and the reference count of plasma cells is less than 1. In some embodiments, wherein after the administration of the anti-programmed cell death protein 1 agent, the anti-programmed death ligand 1 agent, the anti-programmed death ligand 2 agent, or a salt thereof or any combination thereof, a decrease in at least one of the following is observed when compared to a count prior to treatment: a count of T cells, a count of B cells, and a count of plasma cells. In some embodiments, the cancer or the tumor is prostate cancer, testicular cancer, breast cancer, brain cancer, pancreatic cancer, colon cancer, thyroid cancer, stomach cancer, lung cancer, or ovarian cancer. In some embodiments, the method further comprises surgery, radiation therapy, or administering a pharmaceutical agent, wherein the pharmaceutical agent is an immunotherapeutic agent, a chemotherapeutic agent, a targeted therapeutic agent, a hormonal therapy agent, cell-based therapy agent, radiation therapy agent, or any salt thereof, or any combination thereof.

In an aspect, the present disclosure provides a method of treating a cancer or a tumor in a subject, comprising: administering an anti-programmed cell death protein 1 agent, an anti-programmed death ligand 1 agent, an anti-programmed death ligand 2 agent, or a salt thereof or any combination thereof, if a cancer or a tumor sample of a subject contains at least one cell contains a cluster of differentiation 8 (CD8) protein and no cell contains a cluster of differentiation 19 (CD19) protein or a cluster of differentiation 138 (CD138) protein; or administering an anti-programmed cell death protein 1 agent, an anti-programmed death ligand 1 agent, an anti-programmed death ligand 2 agent, or a salt thereof or any combination thereof, and an immunotherapeutic agent or a salt thereof if a cancer or a tumor sample of a subject contains at least one cell contains a CD8 protein and at least one cell contains a CD19 or CD138 protein. In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following is observed when compared to a count prior to treatment: a count of T cells, a count of B cells, and a count of plasma cells. In some embodiments, after the administration of the anti-programmed cell death protein 1 agent, the anti-programmed death ligand 1 agent, the anti-programmed death ligand 2 agent, or a salt thereof or any combination thereof, and an immunotherapeutic agent or a salt thereof, a decrease in at least one of the following is observed when compared to a count prior to treatment: a count of T cells, a count of B cells, and a count of plasma cells. In some embodiments, a decrease in a count of T cells, a decrease in a count of B cells, a decrease in a count of plasma cells, or any combination thereof, is observed.

In some embodiments, the method is a method of treating a cancer or a tumor in the subject. In some embodiments, the cancer or the tumor is prostate cancer, testicular cancer, breast cancer, brain cancer, pancreatic cancer, colon cancer, thyroid cancer, stomach cancer, lung cancer, or ovarian cancer.

In an aspect, the present disclosure provides a method of treating a cancer or a tumor in a subject, comprising administering a therapeutically effective amount of a pharmaceutical agent and an immunotherapeutic agent. In some embodiments, the presence of the CD8 protein, the CD19 protein, the CD20 protein, the CD139 protein, the B-cell activating factor protein, the B-cell activating factor-R protein, and the PAX5 protein is determined by staining, imaging after staining, microscopy, or any combination thereof. In some embodiments, the staining comprises binding of an antibody to the protein In some embodiments, the staining further comprising binding of a second antibody to a first antibody, wherein the second antibody contains a fluorescence marker. In some embodiments, the method is repeated after administration of the therapeutic. In some embodiments, the cancer sample or the tumor sample comprises at least one cell comprising a CD8 protein and at least one cell comprising a CD19 protein, wherein the CD8 protein and the CD19 protein have a distance apart of at most 100 microns. In some embodiments, the cancer sample or the tumor sample comprises at least one cell comprising a CD8 protein and at least one cell comprising a CD19 protein, wherein the CD8 protein-containing cell and the CD19 protein-containing cell have a distance apart of at most 100 microns.

In an aspect, the present disclosure provides a method of treating a solid cancer in a subject in need thereof, comprising: administering a therapeutically effective amount of at least one B-cell activating factor (BAFF) inhibitor and/or at least one B-cell activating factor receptor (BAFF-R) inhibitor, or a salt thereof; and administering a therapeutically effective amount of at least one checkpoint inhibitor or a salt thereof. In some embodiments, the subject is not an appropriate candidate for a monotherapy comprising the at least one checkpoint inhibitor. In some embodiments, the solid cancer is at least partially resistant to a monotherapy comprising the at least one checkpoint inhibitor. In some embodiments, the solid cancer has an incomplete response to a monotherapy comprising the at least one checkpoint inhibitor. In some embodiments, the subject is currently receiving treatment of a monotherapy comprising the at least one checkpoint inhibitor.

In some embodiments, the BAFF inhibitor is tabalumab or a salt thereof. In some embodiments, the BAFF inhibitor is blisibimod or a salt thereof. In some embodiments, the BAFF inhibitor is belimumab or a salt thereof. In some embodiments, the BAFF inhibitor is an anti-BAFF-R antibody or a portion thereof. In some embodiments, the anti-BAFF-R antibody is an anti-BAFF-R3 antibody. In some embodiments, the at least one checkpoint inhibitor or a salt thereof is selected from the group consisting of a monoclonal antibody, a humanized antibody, a chimeric antibody, a fully human antibody, a fusion protein, and any combination thereof. In some embodiments, the checkpoint inhibitor is a biologic or a small molecule. In some embodiments, the checkpoint inhibitor inhibits a checkpoint protein selected from the group consisting of PD1, PDL1, CTLA-4, PDL2, TIM3, LAG3, VISTA, B7-H3, B7-H4, BTLA, HVEM, GAL9, KIR, 2B4, CD160, CGEN-15049, CHK 1, CHK2, A2aR, B-7 family ligands, and any combination thereof. In some embodiments, the checkpoint inhibitor interacts with a ligand of a checkpoint protein selected from the group consisting of PD1, PDL1, CTLA-4, PDL2, TIM3, LAG3, VISTA, B7-H3, B7-H4, BTLA, HVEM, GALS, KIR, 2B4, CD160, CGEN-15049, CHK 1, CHK2, A2aR, B-7 family ligands, and a combination thereof.

In some embodiments, the solid cancer is selected from the group consisting of urogenital, gynecological, lung, gastrointestinal, head and neck cancer, malignant glioblastoma, malignant mesothelioma, non-metastatic or metastatic breast cancer, malignant melanoma, Merkel Cell Carcinoma or bone and soft tissue sarcoma, non-small cell lung cancer (NSCLC), breast cancer, metastatic colorectal cancers, hormone sensitive or hormone refractory prostate cancer, colorectal cancer, ovarian cancer, hepatocellular cancer, renal cell cancer, pancreatic cancer, gastric cancer, esophageal cancer, hepatocellular cancer, cholangiocellular cancer, and small cell lung cancer. In some embodiments, the method further comprises administering an immunotherapeutic agent, a chemotherapeutic agent, or a targeted therapy to the subject prior to, simultaneously with, or after the treating. In some embodiments, the treating is evaluated by a decrease in tumor growth, induction of tumor cell death, tumor regression, preventing or delaying tumor recurrence, preventing or delaying tumor spread, or any combination thereof.

In an aspect, the current disclosure provides a method for treating a cancer in a subject, comprising: screening a cancer sample or a tumor sample isolated from the subject, wherein the screening comprises determining a presence or an absence of a genomic polymorphism or genotype that is correlative to a treatment outcome of the cancer; administering a therapeutically effective amount of at least one B-cell activating factor (BAFF) inhibitor, or a salt thereof, and/or at least one B-cell activating factor receptor (BAFF-R) inhibitor, or a salt thereof; and administering a therapeutically effective amount of at least one PD-L1 inhibitor or a salt thereof, wherein the administering of the at least one B-cell activating factor (BAFF) inhibitor, or salt thereof, and/or the at least one B-cell activating factor receptor (BAFF-R), or salt thereof, and the PD-L1 inhibitor, or salt thereof is based on the screening for the presence or absence of the genomic polymorphism or genotype that is correlative to the treatment outcome of the cancer.

In some embodiments, the PD-L1 inhibitor or a salt thereof is selected from the group consisting of nivolumab, pembrolizumab, pidilizumab, BGB-A31, MEDI0680 (AMP-514), and a salt of any one thereof. In some embodiments, the method further comprises, before the administering: determining a number of an aggregate in the cancer sample or the tumor sample isolated from the subject, wherein the aggregate comprises at least a first cell and a second cell, wherein the first cell and the second cell are of a different cell type, and wherein a population of the first cells and a population of the second cells are in contact with each other.

In some embodiments, the determining of the number of the aggregate is determined by staining, microscopy, imaging after staining, or any combination thereof. In some embodiments, the aggregate comprises at least about 5 cells. In some embodiments, the aggregate comprises about 5 to about 100,000 cells. In some embodiments, the cancer sample or the tumor sample comprises at least 1 aggregate. In some embodiments, the cancer sample or the tumor sample comprises about 1 aggregate to about 20 aggregates. In some embodiments, the aggregate is an immune cell aggregate. In some embodiments, the cell type of the first cell is a lymphoid cell, a plasma cell, a myeloid cell, or a mast cell. In some embodiments, the cell type of the second cell is a lymphoid cell, a plasma cell, a myeloid cell, or a mast cell. In some embodiments, the aggregate is a lymphoid aggregate.

In some embodiments, the aggregate comprises a germinal center. In some embodiments, the aggregate does not comprise a germinal center. In some embodiments, the aggregate is located at an invasive margin, a perivascular space, or a tumor parenchyma of the cancer, or any combination thereof. In some embodiments, the first cell is a B-cell, and wherein the aggregate comprises a B-cell density of at least about 1 cell/mm² by area of the aggregate. In some embodiments, the first cell is a B-cell, and wherein the aggregate comprises a B-cell density of about 1 cell/mm² area of the aggregate to about 1000 cells/mm² by area of the aggregate.

In some embodiments, the first cell is a B-cell, and wherein the aggregate comprises a B-cell density of at least about 100 cells/mm² by area of the aggregate. In some embodiments, the first cell is a B-cell, and wherein the aggregate comprises at least about 1 B-cell. In some embodiments, the first cell is a B-cell, and wherein the aggregate comprises at least about 10 B-cells. In some embodiments, the second cell is a T-cell. In some embodiments, the second cell is a T-cell, and wherein the aggregate comprises at least about 10 T-cells. In some embodiments, the second cell is a T-cell, and wherein the aggregate comprises about 100 T-cells. In some embodiments, the aggregate comprises BAFF and BAFF-R. In some embodiments, the aggregate comprises BAFF and at least one of: CD19, CD20, or CD138. In some embodiments, the first cell or second cell in the aggregate expresses a protein. In some embodiments, the protein is BAFF, BAFF-R, CD3, CD19, CD20, PAX5, or PD-L1. In some embodiments, the determining of the number of the aggregate is assessed by hematoxylin staining, eosin staining, or DAPI staining.

In an aspect, the current disclosure provides a method for selecting a therapeutic regimen for treating a cancer in a subject, the method comprising screening a tissue sample isolated from the subject for evidence of preexisting immunity. In some embodiments, the method further comprises screening the tissue sample for evidence of tumor-induced immunosuppression. In some embodiments, the tumor-induced immunosuppression is BAFF/BAFF-R mediated tumor-induced immunosuppression.

In an aspect, the current disclosure provides a method of detecting a number aggregates in a tissue sample of a subject, the method comprising staining the tissue sample that has been previously diagnosed with a cancer with hemotoxylin, eosin, or DAPI, wherein the staining detects CD3, CD19, CD20, PAX5, BAFF-R, BAFF, or PD-L1, or any combination thereof.

In an aspect, the current disclosure provides a method comprising determining a presence of at least one aggregate of T-cells in a cancer sample or a tumor sample of a subject, wherein the at least one aggregate T-cells is in proximity to at least one B-cell, wherein the B-cell expresses at least one protein selected from the group consisting of: cluster of differentiation 19 (CD19) protein, cluster of differentiation 20 protein (CD20), and cluster of differentiation 138 (CD138) protein. In some embodiments, the determining comprises staining, microscopy, imaging after staining, or any combination thereof. In some embodiments, the aggregate comprises at least about 5 cells. In some embodiments, the aggregate comprises about 5 to about 100,000 cells.

In some embodiments, the cancer sample or the tumor sample comprises at least 1 aggregate. In some embodiments, the aggregate is an immune cell aggregate. In some embodiments, the aggregate is a lymphoid aggregate. In some embodiments, the aggregate comprises a germinal center. In some embodiments, the aggregate does not comprise a germinal center. In some embodiments, the aggregate is located at an invasive margin, a perivascular space, or a tumor parenchyma of the cancer, or any combination thereof. In some embodiments, the method is a method of determining if a cancer or a tumor will be responsive to a single therapeutic agent. In some embodiments, the single therapeutic agent is an anti-programmed cell death protein 1 agent, an anti-programmed death ligand 1 agent, an anti-programmed death ligand 2 agent, or a salt thereof or any combination thereof.

In some embodiments, the at least one aggregate of T-cells is in proximity to at least one B-cell, wherein the B-cell expresses at least one protein selected from the group consisting of: cluster of differentiation 19 (CD19) protein, cluster of differentiation 20 protein (CD20), and cluster of differentiation 138 (CD138) protein. In some embodiments, the method further comprises selecting a therapeutic regimen comprising: an anti-programmed cell death protein 1 agent, an anti-programmed death ligand 1 agent, an anti-programmed death ligand 2 agent, or a salt thereof or any combination thereof; and an therapeutic agent or a salt thereof; wherein the at least one aggregate of T-cells is in proximity to at least one B-cell, wherein the B-cell expresses at least one protein selected from the group consisting of: cluster of differentiation 19 (CD19) protein, cluster of differentiation 20 protein (CD20), and cluster of differentiation 138 (CD138) protein.

In some embodiments, the anti-programmed cell death protein 1 agent or a salt thereof is selected from the group consisting of vemurafenib, erlotinib, docetaxel, nivolumab, pembrolizumab, and a salt of any of these. In some embodiments, the therapeutic agent or a salt thereof is selected from the group consisting of ardenermin, briobacept, blisibimod, belimumab, atransmembrane activator and calcium-modulator and cyclophilin ligand interactorcept, tabalumab, a salt of any of these, and any combination thereof. In some embodiments, at least one of the following proteins are present in the at least one B-cell, the at least one aggregate of T-cells, or both: CD3, CD19, CD20, PAX5, BAFF-R, BAFF, and PD-L1. In some embodiments, the method is a method of diagnosing a cancer or a tumor in the subject. In some embodiments, the method is a method of determining if the cancer or the tumor is becoming non-responsive to a single agent therapy.

In an aspect, the current disclosure provides a method comprising obtaining a cancer sample or a tumor sample of a subject and identifying a presence of a T-cell interacting with at least one cell bearing at least one protein selected from the group consisting of: cluster of differentiation 19 (CD19) protein, cluster of differentiation 20 protein (CD20), and cluster of differentiation 138 (CD138) protein. In some embodiments, the at least one cell is a B-cell. In some embodiments, staining the B-cell detects CD3, CD19, CD20, PAX5, BAFF-R, BAFF, or PD-L1, or any combination thereof.

In an aspect, the current disclosure provides a method of treating a cancer in a subject, comprising: administering a therapeutically effective amount of an immunotherapeutic agent or a salt thereof and a therapeutically effective amount of a checkpoint inhibitor. In some embodiments, the cancer or the tumor comprises a T-cell interacting with at least one cell bearing at least one protein selected from the group consisting of: cluster of differentiation 19 (CD19) protein, cluster of differentiation 20 protein (CD20), and cluster of differentiation 138 (CD138) protein. In some embodiments, the at least one cell is a B-cell. In some embodiments, staining the B-cell detects CD3, CD19, CD20, PAX5, BAFF-R, BAFF, or PD-L1, or any combination thereof.

In some aspects, the current disclosure provides a method comprising: obtaining a cancer sample or a tumor sample from a subject; identifying a presence of B-cell activating factor (BAFF) and B-cell activating factor receptor (BAFF-R) in the cancer sample or the tumor sample; or identifying a presence of B-cell activating factor (BAFF) and at least one protein selected from the group consisting of: cluster of differentiation 19 (CD19) protein, cluster of differentiation 20 protein (CD20), and cluster of differentiation 138 (CD138) protein, in the cancer sample or the tumor sample. In some embodiments, the method further comprises administering a therapeutically effective amount of an anti-programmed cell death protein 1 agent, an anti-programmed death ligand 1 agent, an anti-programmed death ligand 2 agent, or a salt thereof or any combination thereof, and an immunotherapeutic agent or a salt thereof.

In some embodiments, the cancer or the tumor comprises a T-cell interacting with at least one cell bearing at least one protein selected from the group consisting of: cluster of differentiation 19 (CD19) protein, cluster of differentiation 20 protein (CD20), and cluster of differentiation 138 (CD138) protein. In some embodiments, the cancer sample or the tumor sample comprises at least one aggregate of T-cells in proximity to at least one B-cell, wherein the B-cell expresses at least one protein selected from the group consisting of: cluster of differentiation 19 (CD19) protein, cluster of differentiation 20 protein (CD20), and cluster of differentiation 138 (CD138) protein.

In some aspects, the current disclosure provides a method of determining a first amount of cluster of differentiation 8 protein in a cancer sample or a tumor sample of a subject prior to a single agent treatment and determining a second amount of cluster of differentiation 8 protein in the cancer sample or the tumor sample of the subject on or after day 20 of the single agent treatment.

In some aspects, the current disclosure provides a method of selecting combination therapeutic regimen, comprising: selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof wherein the second amount of cluster of differentiation 8 protein in the cancer sample or the tumor sample of the subject on or after day 20 of the single agent treatment is higher than the first amount of cluster of differentiation 8 protein in the cancer sample or the tumor sample of the subject prior to the single agent treatment.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “figure” and “FIG.” herein), of which:

FIG. 1 provides a representative example of a matrix that defines cellular boundaries;

FIG. 2 provides a 9×9 representative example which corresponds with FIG. 1 and is an example of an intensity matrix, wherein the relative intensity of each marker of interest per pixel is defined;

FIG. 3. provides an example of pixel labeling;

FIG. 4 provides frequency polygons of pixel staining intensities;

FIG. 5 shows an example of positive intensity thresholding with heatmap visualization;

FIG. 6. provides an example of an adjacency graph;

FIG. 7 parts A-C provides an example heatmap;

FIG. 8 shows an adjacency heatmap of a subject that did not respond to treatment, or a non-responder subject;

FIG. 9 shows a fraction of adjacency heatmap;

FIG. 10 shows an adjacency heatmap of the difference between the heatmap of a responder minus the heatmap of a non-responder;

FIG. 11 illustrates the use of pixel-based analytics for diagnostic and therapeutic development;

FIG. 12 shows a computer control system;

FIGS. 13A-13C and FIGS. 14A-14C illustrate three types of tumors before and after treatment, including Type II resistant tumors;

FIGS. 15A-15B illustrate the proliferation of Type II resistant tumors;

FIG. 16 illustrates the proliferation of Type I and Type II resistant tumors;

FIG. 17 illustrates the Type II resistant tumors are associated with high PD-L1 expression at baseline;

FIG. 18 illustrates BAFF expression in samples obtained before anti-PD1 therapy according to clinical response;

FIG. 19 illustrates BAFF-R expression in samples obtained before anti-PD1 therapy according to clinical response; and

FIG. 20 illustrates BAFF/BAFF-R expression in breast cancer, non-small cell lung cancer (squamous), non-small cell lung cancer (adeno), and head and neck squamous cell carcinoma.

### DETAILED DESCRIPTION

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes: (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar, (14) buffering agents, such as magnesium hydroxide and aluminum hydroxide; (15) alginic acid; (16) pyrogen-free water; (17) isotonic saline; (18) Ringer's solution; (19) ethyl alcohol: (20) phosphate buffer solutions; and (21) other non-toxic compatible substances employed in pharmaceutical formulations.

In certain embodiments, the term “prevent” or “preventing” as related to a disease or disorder may refer to a compound or composition that, in a statistical sample, reduces the occurrence of the disorder or condition in the treated sample relative to an untreated control sample, or delays the onset or reduces the severity of one or more symptoms of the disorder or condition relative to the untreated control sample.

The terms “treat,” “treating” or “treatment,” as used herein, may include at least partially: alleviating, abating or ameliorating a disease or condition symptom, preventing an additional symptom, ameliorating or preventing the underlying causes of symptom, inhibiting the disease or condition, e.g., at least partially arresting the development of the disease or condition, relieving the disease or condition, causing regression of the disease or condition, relieving a condition caused by the disease or condition, or stopping a symptom of the disease or condition either prophylactically and/or therapeutically. In some embodiments, treatment can include stopping the growth of a cancer, shrinking the mass of a cancer, shrinking the volume of a cancer, and prolonging the life span of a subject when compared to an otherwise substantially identical subject who is not treated.

Included in the present disclosure are salts, including pharmaceutically acceptable salts, of the compositions described herein. The compounds or compositions of the present disclosure that possess a sufficiently acidic, a sufficiently basic, or both functional groups, can react with any of a number of inorganic bases, and inorganic and organic acids, to form a salt. Alternatively, compositions containing compounds that are inherently charged, such as those with a quaternary nitrogen, can form a salt with an appropriate counterion, e.g., a halide such as bromide, chloride, or fluoride, particularly bromide.

As used herein, “agent” or “biologically active agent” can refer to a biological, pharmaceutical or chemical compound or a salt of any of these. Non-limiting examples include a simple or complex organic or inorganic molecule, a peptide, a protein, an oligonucleotide, an antibody, an antibody derivative, antibody fragment, a vitamin derivative, a carbohydrate, a toxin, and a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like.

As used herein, an “immunotherapeutic agent” can refer to an agent that may be used on or used to modify an immune mechanism or immune response.

As used herein, a “pharmaceutical agent” can refer to an agent or a therapy that may be used to prevent, diagnose, treat, or cure a disease, or combinations thereof.

The methods and compositions described herein include the use of amorphous forms as well as crystalline forms or polymorphs. The compounds described herein may be in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

### Methods

In some embodiments, a method of the current disclosure comprises administering a therapeutically effective amount of an immunotherapeutic agent or a salt thereof, wherein the immunotherapeutic agent or a salt thereof inhibits a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof; wherein the immunotherapeutic agent is co-administered with a checkpoint inhibitor.

In some embodiments, a method of the current disclosure comprises treating a subject in need thereof, comprising: administering a therapeutically effective amount of an immunotherapeutic agent or a salt thereof, wherein the immunotherapeutic agent or a salt thereof inhibits a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof; and administering a checkpoint inhibitor.

In some embodiments, a method of treatment of the current disclosure results in at least partial remission of a cancer or a tumor. After administration of a composition described herein, progression of a cancer or a tumor may be delayed. Methods described herein may delay progression of a cancer or a tumor. After administration of a composition, the mass of a cancer or a tumor may be reduced, or the volume of a cancer or a tumor may be reduced. A method described herein may prolong the lifespan, the expected lifespan, or the life of a subject.

In one aspect, the present disclosure provides a method of treating a subject in need thereof, comprising: administering a therapeutically effective amount of at least one B-cell activating factor (BAFF) inhibitor and/or at least one B-cell activating factor receptor (BAFF-R) inhibitor, or a salt thereof; and administering a therapeutically effective amount of at least one checkpoint inhibitor or a salt thereof. In some embodiments, the subject has been diagnosed with a cancer or a tumor. In some embodiments, the method is a method of treating a cancer or a tumor in the subject. In some embodiments, the subject has not previously undergone treatment for the cancer or the tumor. In some embodiments, the subject has previously or is currently undergoing treatment for the cancer or the tumor. In some embodiments, the subject has previously undergone treatment with surgery or radiation, and the subject remains in remission. In some embodiments, the subject has previously undergone treatment with surgery, radiation, an anticancer agent, and any combination thereof, and the cancer or the tumor was at least partially refractive to the treatment. In some embodiments, the subject was previously treated with surgery, radiation therapy, an anticancer agent, and any combination thereof, and the cancer or the tumor re-occurred in the subject following a period of remission, wherein the period of remission was at least about: 1 month, 2 months, 3 months, 6 months, 1 year, 2 years, 3 years, 4 years, 5 years, 6 years, 7 years, 8 years, 9 years, or 10 years.

In some embodiments, the subject was previously treated with a checkpoint inhibitor or a salt thereof, and the cancer or the tumor was at least partially refractive to the checkpoint inhibitor or a salt thereof. In some embodiments, the subject was previously treated with a checkpoint inhibitor or a salt thereof, and the cancer or the tumor re-occurred in the subject following a period of remission, wherein a period of the remission was at least about: 1 month, 2 months, 3 months, 6 months, 1 year, 2 years, 3 years, 4 years, 5 years, 6 years, 7 years, 8 years, 9 years, or 10 years. In some embodiments, the checkpoint inhibitor or a salt thereof is administered sequentially with the BAFF inhibitor and/or BAFF-R inhibitor or a salt thereof. In some embodiments, the checkpoint inhibitor or a salt thereof is administered concurrently with the BAFF inhibitor and/or BAFF-R inhibitor or a salt thereof. In some embodiments, the checkpoint inhibitor is selected from the group consisting of an agent that binds to anti-programmed cell death protein 1 (PD-1), anti-programmed death ligand 1 (PD-L1), anti-programmed death ligand 2 (PD-L2), cytotoxic T-lymphocyte-associated protein 4 (CTLA4), cluster of differentiation 276 (B7-H3), V-set domain-containing T-cell activation inhibitor 1 (B7-H4), T-cell immunoglobulin and mucin-domain containing-3 (TIM-3), lymphocyte-activation gene 3 (LAG3), Indoleamine-pyrrole 2,3-dioxygenase (IDO), a salt of any one thereof, and any combination thereof.

In some embodiments, the BAFF-inhibitor may comprise the sequence of any of SEQ ID NOs: 1-16.

SEQ ID NO: 1→polynucleotide sequence encoding
light chain variable region
GAAATTGTGTTGACGCAGTCTCCAGCCACCCTGTCTTTGTCTCCAGGGGA
AAGAGCCACCCTCTCCTGCAGGGCCAGTCAGAGTGTTAGCCGCTACTTAG
CCTGGTACCAGCAGAAACCTGGCCAGGCTCCCAGGCTCCTCATCTATGAT
GCATCCAACAGGGCCACTGGCATCCCAGCCAGGTTCAGTGGCAGTGGGTC
TGGGACAGACTCCACTCTCACCATCAGCAGCCTAGAGCCTGAAGATTTTG
CAGTTTATTACTGTCAGCAGCGTAGCAACTGGCCTCGGACGTTCGGCCAA
GGGACCAAGGTGGAAATCAAACGAACT
SEQ ID NO: 2→amino acid sequence encoding light
chain variable region
EIVLTQSPATLSLSPGERATLSCRASQSVSRYLAWYQQKPGQAPRLLIYD
ASNRATGIPARFSGSGSGTDSTLTISSLEPEDFAVYYCQQRSNWPRTFGQ
GTKVEIKRT
SEQ ID NO: 3→polynucleotide sequence encoding
light chain CDR1
AGGGCCAGTCAGAGTGTTAGCCGCTACTTAGCC
SEQ ID NO: 4→amino acid sequence encoding light
chain CDR1
RASQSVSRYLA
SEQ ID NO: 5→polynucleotide sequence encoding
light chain CDR2
GATGCATCCAACAGGGCCACT
SEQ ID NO: 6→amino acid sequence encoding
light chain CDR2
DASNRAT
SEQ ID NO: 7→polynucleotide sequence encoding
light chain CDR3
CAGCAGCGTAGCAACTOGCCICGOACG
SEQ ID NO: 8→amino acid sequence encoding
light chain CDR3
QQRSNWPRT
SEQ ID NO: 9→polynucleotide sequence encoding
heavy chain variable region
ATGAAA
CACCTGTGGTTCTTCCTCCTCCTGGTGGCAGCTCCCAGATGGGTCCTGTC
CCAGGTGCAACTACAGCAGTGGGGCGCAGGACTGTTGAAGCCTTCGGAGA
CCCTGTCCCTCACCTGCGCTGTCTATGGTGGGTCCTTCAGTGGTTACTAC
TGGAGCTGGATCCGCCAGCCCCCAGGGAAGGGGCTGGAGTGGATTGGGGA
AATCAATCATAGTGGAAGCACCAACTACAACCCGTCCCTCAAGAGTCGAG
TCACCATATCAGTAGACACGTCCAAGAACCAGTTCTCCCTGAAACTGAGC
TCTGTGACCGCCGCGGACACGGCTGTGTATTACTGTGCGAGAGGGTATTA
CGATATTTTGACTGGTTATTATTACTACTTTGACTACTGGGGCCAGGGAA
CCCTGGTCACCGTCTCCTCA
SEQ ID: 10→amino acid sequence encoding heavy
chain variable region
MKHLWFFLLLVAAPRWVLSQVQLQQWGAGLLKPSETLSLTCAVYGGSFSG
YYWSWIRQPPGKGLEWIGEINHSGSTNYNPSLKSRVTISVDTSKNQFSLK
LSSVTAADTAVYYCARGYYDILTGYYYYFDYWGQGTLVTVSS
SEQ ID NO: 11→polynucleotide sequence encoding
heavy chain CDR1
GGTGGGTCCTTCAGTGGTTACTACTGGAGC
SEQ ID NO: 12→amino acid sequence encoding heavy
chain CDR1
GGSFSGYYWS
SEQ ID NO: 13→polynucleotide sequence encoding
heavy chain CDR2
GAAATCAATCATAGTGGAAGCACCAACTACAACCCGTCCCTCAAGAGT
SEQ ID NO: 14→amino acid sequence encoding heavy
chain CDR2
EINHSGSTNYNPSLKS
SEQ ID NO: 15→polynucleotide sequence encoding
heavy chain CDR3
GGGTATTACGATATTTTGACTGGTTATTATTACTACTTTGACTAC
SEQ ID NO: 16 amino acid sequence encoding heavy
chain CDR3
GYYDILTGYYYYFDY

In some embodiments, the checkpoint inhibitor or a salt thereof is a PD-1 inhibitor or a salt thereof. In some embodiments, the PD-1 inhibitor or a salt thereof is selected from the group consisting of nivolumab, pembrolizumab, pidilizumab, BGB-A31, MEDI0680 (AMP-514), and a salt of any one thereof. In some embodiments, the PD-1 inhibitor is nivolumab or a salt thereof. In some embodiments, the PD-1 inhibitor is pembrolizumab or a salt thereof. In some embodiments, the PD-1 inhibitor is pidilizumab or a salt thereof. In some embodiments, the PD-1 inhibitor is BGB-A31 or a salt thereof. In some embodiments, the PD-1 inhibitor is MEDI0680 (AMP-514) or a salt thereof.

In some embodiments, the checkpoint inhibitor or a salt thereof is a PD-L1 inhibitor or a salt thereof. In some embodiments, the PD-L inhibitor or a salt thereof is selected from the group consisting of atezolizumab, avelumab, durvalumab, MDX-1105, MSB0010718C, and a salt of any one thereof. In some embodiments, the PD-L1 inhibitor is atezolizumab or a salt thereof. In some embodiments, the PD-L1 inhibitor is avelumab or a salt thereof. In some embodiments, the PD-L1 inhibitor is durvalumab or a salt thereof. In some embodiments, the PD-L1 inhibitor is MDX-1105 or a salt thereof. In some embodiments, the PD-L1 inhibitor is MSB0010718C or a salt thereof.

In some embodiments, the checkpoint inhibitor is a CTLA4 inhibitor or a salt thereof. In some embodiments, the CTLA4 inhibitor or a salt thereof is selected from the group consisting of ipilimumab, tremelimumab, AGEN1884, and a salt of any one thereof. In some embodiments, the CTLA4 inhibitor is ipilimumab or a salt thereof. In some embodiments, the CTLA4 inhibitor is tremelimumab or a salt thereof. In some embodiments, the CTLA4 inhibitor is AGEN1884 or a salt thereof.

In some embodiments, the checkpoint inhibitor or a salt thereof is a LAG3 inhibitor or a salt thereof. In some embodiments, the LAG3 inhibitor is BMS-986016 or a salt thereof. In some embodiments, the checkpoint inhibitor or a salt thereof is a TIM3 inhibitor or a salt thereof. In some embodiments, the TIM3 inhibitor or a salt thereof is selected from the group consisting of MBG453, TSR-022, and a salt of any one thereof. In some embodiments, the TIM3 inhibitor is MBG453 or a salt thereof. In some embodiments, the TIM3 inhibitor is TSR-022 or a salt thereof.

Another aspect of the current disclosure provides a method of treating a subject in need thereof, comprising: administering a therapeutically effective amount of at least one BAFF and/or BAFF-R inhibitor or a salt thereof; and administering at least one immune agonist agent or a salt thereof. In some embodiments, the immune agonist agent or a salt thereof is selected from the group consisting of an agent that binds to glucocorticoid-induced tumor necrosis factor receptor-related protein (GITR), cluster of differentiation 134 (OX40), cluster of differentiation 137 (CD137), cluster of differentiation 40 (CD40), Toll-like receptor (TLR), or a salt of any one thereof, and any combination thereof.

In some embodiments, the immune agonist agent is a GITR agonist or a salt thereof. In some embodiments, the GITR agonist or a salt thereof is selected from the group consisting of an agent that hinds to TRX518, GWN323, MEDI1873, INCAGN01876, and a salt of any one thereof. In some embodiments, the GITR agonist is TRX518 or a salt thereof. In some embodiments, the GITR agonist is GWN323 or a salt thereof. In some embodiments, the GITR agonist is MEDI1873 or a salt thereof. In some embodiments, the GITR agonist is INCAGN01876 or a salt thereof.

In some embodiments, the immune agonist agent is an OX40 agonist or a salt thereof. In some embodiments, the OX40 agonist is selected from the group consisting of GSK3174998, PF-04518600, MEDI6469, INCAGN01949, and a salt of any one thereof. In some embodiments, the OX40 agonist is GSK3174998 or a salt thereof. In some embodiments, the OX40 agonist is PF-04518600 or a salt thereof. In some embodiments, the OX40 agonist is MEDI6469 or a salt thereof. In some embodiments, the OX40 agonist is INCAGN01949 or a salt thereof.

In some embodiments, the immune agonist agent is a cluster of differentiation 137 (4-1BB) agonist or a salt thereof. In some embodiments, the 4-1BB agonist is urelumab, utomilumab, or a salt of any one thereof. In some embodiments, the 4-1BB agonist is isurelumab or a salt thereof. In some embodiments, the 4-1BB agonist is isutomilumab or a salt thereof.

In some embodiments, the immune agonist agent is a CD40 agonist or a salt thereof. In some embodiments, the CD40 agonist is APX005M, CP870893, or a salt thereof. In some embodiments, the CD40 is APX005M or a salt thereof. In some embodiments, the CD40 is CP870893 or a salt thereof.

In some embodiments, the immune agonist agent is a TLR agonist or a salt thereof. In some embodiments, the TLR agonist is selected from the group consisting of an agent that binds to TLR-1, TLR-2, TLR-3, TLR-4, TLR-5, TLR-6, TLR-7, TLR-8, TLR-9, TLR-13, a salt or any one thereof, and any combination thereof.

Yet another aspect of the current disclosure provides a method of treating a subject in need thereof, comprising: administering a therapeutically effective amount of at least one BAFF and/or BAFF-R inhibitor or a salt thereof; and administering at least one vaccine agent or a salt thereof. In some embodiments, the vaccine agent is selected from the group consisting of MAGE-3, NY-ESO-1, TRAG-3, p53, at least one or more a-actinin-4 and malic enzymes, carcinoembryonic antigen, HER2, MUC1, survivin, WT-1, PRAME, Survivin-2b, Bacillus Calmette-Guerin, MVAX, at least one or more heat shock proteins, keyhole limpet hemocyanin, interleukin-2, QS21, montanide ISA-51, granulocyte monocyte-colony stimulating factor, GVAX, GI-4000, CDX-1307, IMA910, TroVAX, CRS-207, CA-9, a salt of any one thereof, and any combination thereof.

An aspect of the current disclosure provides a method of treating a subject in need thereof, comprising: administering a therapeutically effective amount of at least one BAFF and/or BAFF-R inhibitor or a salt thereof; and administering at least one oncolytic viral based agent or a salt thereof. In some embodiments, the oncolytic viral based agent is selected from the group consisting of enadenotucirev, talimogene laherparepvec, reolysin, CG0070, Pexastimogene devacirepvec, cavatak, oncolytic vesicular stomatitis virus, ONCOS-102, a salt of any one thereof, and any combination thereof.

An aspect of the current disclosure provides a method of treating a subject in need thereof, comprising: administering a therapeutically effective amount of at least one BAFF and/or BAFF-R inhibitor or a salt thereof; and administering at least one cell-based therapy, any cell derivative thereof, or a salt of any one thereof. In some embodiments, the cell-based therapy is selected from the group consisting of: at least one or more autologous lymphocytes, at least one or more genetically engineered autologous lymphocytes, at least one or more chimeric antigen receptor cells, at least one or more chimeric antigen receptor T-cells, at least one or more dendritic cell based vaccines, a salt of any one thereof, and any combination thereof.

Another aspect of the current disclosure provides a method of treating a subject in need thereof, comprising: administering a therapeutically effective amount of at least one BAFF and/or BAFF-R inhibitor or a salt thereof; and administering at least one chemotherapeutic agent or a salt thereof. In some embodiments, the chemotherapeutic agent is an alkylating agent, an antimetabolite agent, a plant alkaloid agent, an antitumor antibiotic, a salt of any one thereof, and any combination thereof.

One aspect of the current disclosure provides for a method of treating a subject in need thereof, comprising: administering a therapeutically effective amount of at least one BAFF and/or BAFF-R inhibitor or a salt thereof; and administering at least one targeted therapeutic agent or a salt thereof. In some embodiments, the targeted therapeutic agent is an agent that inhibits signal transduction, angiogcncsis, hormone expression, or any combination thereof. In some embodiments, the BAFF inhibitor and/or BAFF-R inhibitor prevents binding of BAFF or a BAFF-induced ligand to a BAFF-R, is a partial antagonist of a BAFF, is a partial agonist of a BAFF, is a competitive antagonist of a BAFF-R, is a non-competitive antagonist of a BAFF-R receptor, or any combination thereof. In some embodiments, the BAFF inhibitor is an antibody, antigen binding fragment, a bispecific antibody, or a recombinant fusion protein.

In some embodiments, the BAFF inhibitor antibody is human, humanized, chimeric, composite, polyclonal or monoclonal. In some embodiments, the BAFF antibody or antigen binding fragment blocks interaction of molecules to BAFF. In some embodiments, the BAFF inhibitor is selected from the group consisting of tabalumab. BAFF/IL-17 bispecific antibody, ardenermin, briobacept, blisibimod, belimumah, a transmembrane activator and calcium-modulator and cyclophilin ligand interactorcept, a salt of any one thereof, and any combination thereof. In some embodiments, the BAFF inhibitor is selected from the group consisting of MEDI-700, NOV-5, rGel/BLyS, a salt of any one thereof, and any combination thereof. In some embodiments, the BAFF inhibitor is tabalumab or a salt thereof. In some embodiments, the BAFF inhibitor is blisibimod or a salt thereof. In some embodiments, the BAFF inhibitor is belimumab or a salt thereof.

One aspect of the current disclosure provides a method of treating a subject in need thereof, comprising: administering a therapeutically effective amount of an immunotherapeutic agent or a salt thereof, wherein the immunotherapeutic agent or a salt thereof inhibits a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof; wherein the immunotherapeutic agent is co-administered with a checkpoint inhibitor.

In some embodiments, the subject has been diagnosed with a cancer or a tumor. In some embodiments, the method is a method of treating a cancer or a tumor in the subject. In some embodiments, the subject has previously or is currently undergoing treatment for the cancer or the tumor. In some embodiments, the subject was previously treated with a checkpoint inhibitor, and the cancer or the tumor was at least partially refractive to the checkpoint inhibitor. In some embodiments, a method comprises administering a therapeutically effective amount of the checkpoint inhibitor.

In some embodiments, the checkpoint inhibitor is a salt. In some embodiments, the checkpoint inhibitor is selected from the group consisting of an anti-programmed cell death protein 1 agent, an anti-programmed death ligand 1 agent, an anti-programmed death ligand 2 agent, a salt of any of these, and any combination thereof.

In some embodiments, a method comprises administering the anti-programmed cell death protein 1 agent or a salt thereof. In some embodiments, the anti-programmed cell death protein 1 agent or a salt thereof is selected from the group consisting of vemurafenib, erlotinib, docetaxel, nivolumab, pembrolizumab, and a salt of any of these. In some embodiments, the anti-programmed cell death protein 1 agent or a salt thereof is vemurafenib. In some embodiments, the anti-programmed cell death protein 1 agent or a salt thereof is erlotinib. In some embodiments, the anti-programmed cell death protein 1 agent or a salt thereof is pembrolizumab. In some embodiments, a method comprises administering the anti-programmed death ligand 1 agent or a salt thereof.

In some embodiments, the anti-programmed death ligand 1 agent or a salt thereof is selected from the group consisting of atezolizumab, avelumab, durvalumab, and a salt of any of these. In some embodiments, the immunotherapeutic agent or a salt thereof at least partially prevents binding of a ligand to a B-cell activating factor receptor, is a partial antagonist of a B-cell activating factor receptor, is a partial agonist of a B-cell activating factor receptor, is a competitive antagonist of a B-cell activating factor receptor, is a non-competitive antagonist of a B-cell activating factor receptor, or any combination thereof. In some embodiments, the immunotherapeutic agent or a salt thereof at least partially prevents ligand binding to a transmembrane activator and calcium-modulator and cyclophilin ligand interactor receptor, is a partial antagonist of a transmembrane activator and calcium-modulator and cyclophilin ligand interactor receptor, is a partial agonist of a transmembrane activator and calcium-modulator and cyclophilin ligand interactor receptor, or any combination thereof.

In some embodiments, the immunotherapeutic agent or a salt thereof at least partially prevents ligand binding to a B-cell maturation antigen receptor, is a partial antagonist of a B-cell maturation antigen receptor, is a partial agonist of a B-cell maturation antigen receptor, or any combination thereof. In some embodiments, the immunotherapeutic agent is an antibody or a salt thereof. In some embodiments, the immunotherapeutic agent is a human or humanized monoclonal antibody or a salt thereof. In some embodiments, the immunotherapeutic agent or a salt thereof is selected from the group consisting of ardenermin, briobacept, blisibimod, belimumab, atransmembrane activator and calcium-modulator and cyclophilin ligand interactorcept, tabalumab, a salt of any of these, and any combination thereof.

In some embodiments, the immunotherapeutic agent or a salt thereof is selected from the group consisting of MEDI-700, NOV-5, rGel/BLyS, a salt of any of these, and any combination thereof. In some embodiments, the immunotherapeutic agent is tabalumab or a salt thereof. In some embodiments, the immunotherapeutic agent is blisibimod or a salt thereof. In some embodiments, the immunotherapeutic agent is belimumab or a salt thereof.

In some embodiments, a method described herein further comprising, before the administering: determining a count of cluster of differentiation 8 (CD8) protein, and at least one selected from the group consisting of: cluster of differentiation 19 (CD19) protein, cluster of differentiation 20 protein (CD20), cluster of differentiation 138 (CD138) protein, B-cell activating factor protein, B-cell activating factor receptor protein, and paired box Pax-5 (PAX5) protein, in a cancer or a tumor or a cancer sample or a tumor sample of a subject.

In some embodiments, the at least one of: CD19, CD20, CD138, B-cell activating factor, B-cell activating factor-R, and PAX5 is present in the cancer or the tumor or the cancer sample or the tumor sample of the subject. In some embodiments, the at least one of: CD19, CD20, CD138, B-cell activating factor, B-cell activating factor-R, and PAX5 is individually present in the cancer or the tumor or the cancer sample or the tumor sample of the subject in an amount ranging from 1 protein to at least about 10,000,000 proteins.

In some embodiments, a method described herein further comprising monitoring the treatment after the administering. In some embodiments, the treatment maintains at least partial remission of the cancer or the tumor, wherein a period of the remission was at least about: 1 month, 2 months, 3 months, 6 months, 1 year, 2 years, 3 years, 4 years, 5 years, 6 years, 7 years, 8 years, 9 years, or 10 years. In some embodiments, the treatment comprises, after administering, delaying progression of the cancer or the tumor in the subject. In some embodiments, the treatment comprises, after administering, regression of the cancer or the tumor.

In some embodiments, the regression is a reduction in mass of the cancer or the tumor, a reduction in volume of the cancer or the tumor, or both. In some embodiments, the treatment comprises prolonging the subject's life. In some embodiments, the subject has previously shown at least partial refraction to a monotherapy for the cancer or the tumor. In some embodiments, the subject has shown refraction to a monotherapy for the cancer or the tumor. In some embodiments, the cancer has metastasized from a first location of the subject to a second location of the subject. In some embodiments, the cancer at the first location of the body is less than fully responsive to the monotherapy, and wherein the cancer at the second location of the body is responsive to the treatment. In some embodiments, the checkpoint inhibitor and the immunotherapeutic agent are synergistic. In some embodiments, the synergy on the cancer or the tumor is at least 10% more than an additive effect.

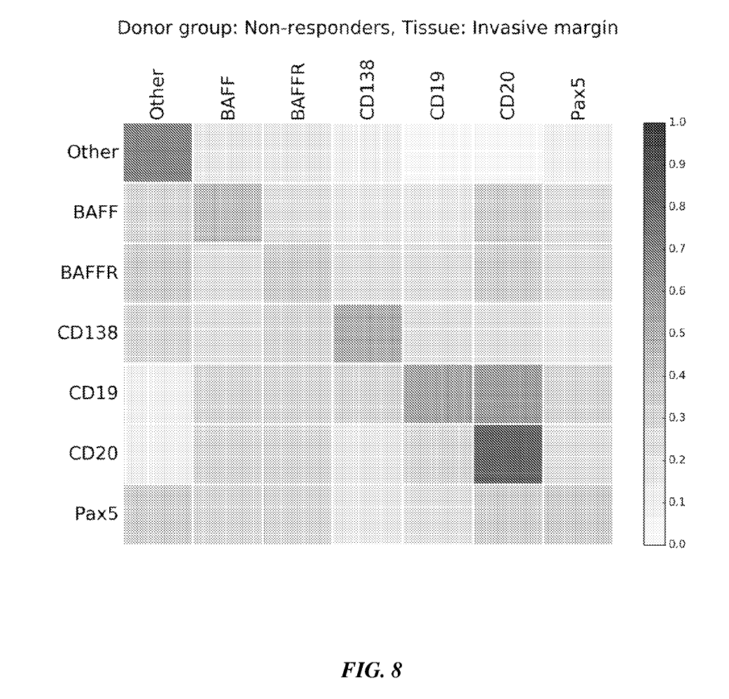

In some embodiments, a heatmap of a cancer sample or a tumor sample of the subject shows a profile substantially similar to that of FIG. 8.

In some embodiments, the anti-programmed cell death protein 1 agent, the anti-programmed death ligand 1 agent, the anti-programmed death ligand 2 agent, or a salt thereof or any combination thereof, and the immunotherapeutic agent or a salt thereof are administered concurrently. In some embodiments, the anti-programmed cell death protein 1 agent, the anti-programmed death ligand 1 agent, the anti-programmed death ligand 2 agent, or a salt thereof or any combination thereof, and the immunotherapeutic agent or a salt thereof are administered sequentially. In some embodiments, the anti-programmed cell death protein 1 agent, the anti-programmed death ligand 1 agent, the anti-programmed death ligand 2 agent, or a salt thereof or any combination thereof, and the immunotherapeutic agent or a salt thereof are administered in different formulations within a same treatment schedule.

In some embodiments, the anti-programmed cell death protein 1 agent, the anti-programmed death ligand 1 agent, the anti-programmed death ligand 2 agent, or a salt thereof or any combination thereof, is administered at least once a week. In some embodiments, the anti-programmed cell death protein 1 agent, the anti-programmed death ligand 1 agent, the anti-programmed death ligand 2 agent, or a salt thereof or any combination thereof, is administered at least once a day. In some embodiments, the anti-programmed cell death protein 1 agent, the anti-programmed death ligand 1 agent, the anti-programmed death ligand 2 agent, or a salt thereof or any combination thereof, is administered at least once during a treatment schedule.

In some embodiments, the immunotherapeutic agent or a salt thereof is administered at least once a week. In some embodiments, the immunotherapeutic agent or a salt thereof is administered at least once a day. In some embodiments, the immunotherapeutic agent or a salt thereof is administered at least once during a treatment schedule. In some embodiments, the immunotherapeutic agent or a salt thereof is administered in an amount from about 0.1 mg to about 1,000 mg per kg body weight. In some embodiments, the immunotherapeutic agent or a salt thereof is administered in an amount from about 0.1 mg to about 100 mg per kg body weight. In some embodiments, the immunotherapeutic agent or a salt thereof is administered in an amount from about 1 mg to about 50 mg per kg body weight.

In some embodiments, the anti-programmed cell death protein 1 agent, the anti-programmed death ligand 1 agent, the anti-programmed death ligand 2 agent, or a salt thereof or any combination thereof, is administered in an amount from about 0.1 mg to about 1,000 mg per kg body weight. In some embodiments, the anti-programmed cell death protein 1 agent, the anti-programmed death ligand 1 agent, the anti-programmed death ligand 2 agent, or a salt thereof or any combination thereof, is administered in an amount from about 1 mg to about 100 mg per kg body weight. In some embodiments, the anti-programmed cell death protein 1 agent, the anti-programmed death ligand 1 agent, the anti-programmed death ligand 2 agent, or a salt of any one thereof, or any combination thereof, are individually present as a pharmaceutical formulation. In some embodiments, the pharmaceutical formulation is in unit dose form. In some embodiments, the pharmaceutical formulation further comprises a pharmaceutically-acceptable excipient.

The method of any one of the preceding claims, comprising treating the cancer, wherein the cancer is prostate cancer, testicular cancer, breast cancer, brain cancer, pancreatic cancer, colon cancer, thyroid cancer, stomach cancer, lung cancer, melanoma, multiple myeloma. Hodgkin's lymphoma, or ovarian cancer.

In one aspect, the present disclosure provides a method of selecting a therapeutic regimen, comprising: determining the presence or absence of cluster of differentiation 8 (CD8) protein, and at least one of: B-cell activating factor (BAFF) protein, B-cell activating factor receptor (BAFF-R) protein, and paired box Pax-5 (PAX5) protein in a cancer or a tumor of a subject or a cancer sample or a tumor sample of the subject; if CD8 protein and at least one of B-cell activating factor, B-cell activating factor-R, and PAX5 are present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof; if CD8 protein and at least one of B-cell activating factor, B-cell activating factor-R, and PAX5 is not present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof.

In some embodiments, the method further comprises determining whether cluster of differentiation 19 (CD19) protein, cluster of differentiation 20 (CD20) protein, cluster of differentiation 138 (CD138) protein, or any combination thereof is present. In some embodiments, the method further comprises treating the subject.

In some embodiments, the treating comprises: administering a therapeutically effective amount of an immunotherapeutic agent or a salt thereof, wherein the immunotherapeutic agent or a salt thereof inhibits a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof; wherein the immunotherapeutic agent is co-administered with a checkpoint inhibitor.

In some embodiments, the subject has been diagnosed with a cancer or a tumor. In some embodiments, the subject has previously or is currently undergoing treatment for the cancer or the tumor. In some embodiments, the method further comprises administering a therapeutically effective amount of the checkpoint inhibitor.

In some embodiments, the checkpoint inhibitor is selected from the group consisting of an anti-programmed cell death protein 1 agent, an anti-programmed death ligand 1 agent, an anti-programmed death ligand 2 agent, a salt of any of these, and any combination thereof. In some embodiments, a method further comprises administering the anti-programmed cell death protein 1 agent of a salt thereof.

In some embodiments, the anti-programmed cell death protein 1 agent or a salt thereof is selected from the group consisting of vemurafenib, erlotinib, docetaxel, nivolumab, pembrolizumab, and a salt of any of these. In some embodiments, the anti-programmed cell death protein 1 agent or a salt thereof is vemurafenib. In some embodiments, the anti-programmed cell death protein 1 agent or a salt thereof is erlotinib. In some embodiments, the anti-programmed cell death protein 1 agent or a salt thereof is pembrolizumab. In some embodiments, the immunotherapeutic agent is tabalumab or a salt thereof. In some embodiments, the immunotherapeutic agent is belimumab or a salt thereof.

In some embodiments, the cancer or the tumor is prostate cancer, testicular cancer, breast cancer, brain cancer, pancreatic cancer, colon cancer, thyroid cancer, stomach cancer, lung cancer, or ovarian cancer. In some embodiments, the cancer or the tumor contacts a blood vessel.

In some embodiments, the cancer or the tumor is in the interior of a blood vessel.

In one aspect, the present disclosure provides a method of selecting a therapeutic regimen, comprising: determining a count of B cells, T cells, and plasma cells in a cancer or a tumor of a subject or a cancer sample or a tumor sample of the subject; if a count of T cells in the cancer or the tumor or the cancer sample or the tumor sample is greater than 500 cells/mm2 and a count of B cells is less than <150 cells/mm2, selecting a therapeutic regimen comprising an anti-programmed cell death protein 1 agent, an anti-programmed death ligand 1 agent, an anti-programmed death ligand 2 agent, or a salt thereof or any combination thereof; if a count of T cells in the cancer or the tumor or the cancer sample or the tumor sample is greater than 300 cells/mm2 and a count of B cells, a count of plasma cells, or a combination of B cells and plasma cells is greater than 100 cells/mm2, selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof.

In some embodiments, a method further comprises conveying a result via a communication medium.

In one aspect, the current disclosure provides a method of treating or maintaining remission of a cancer or a tumor in a subject, comprising: administering an anti-programmed cell death protein 1 agent, an anti-programmed death ligand 1 agent, an anti-programmed death ligand 2 agent, or a salt thereof or any combination thereof, if a percentage of T cells in a cancer sample or a tumor sample of a subject is greater than 10% and a percentage of B cells is less than 5%, or administering an anti-programmed cell death protein 1 agent, an anti-programmed death ligand 1 agent, an anti-programmed death ligand 2 agent, or a salt thereof or any combination thereof, and an immunotherapeutic agent or a salt thereof if a percentage of T cells in a cancer or a tumor sample of the subject is less than 10% and a percentage of B cells, a percentage of plasma cells, or a combination of the percentage of B cells and the percentage of plasma cells is greater than 10% in the cancer sample of the subject.

In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following is observed when compared to a count prior to treatment: a percentage of T cells, a percentage of B cells, and a percentage of plasma cells. In some embodiments, the cancer or the tumor is prostate cancer, testicular cancer, breast cancer, brain cancer, pancreatic cancer, colon cancer, thyroid cancer, stomach cancer, lung cancer, or ovarian cancer.

In some embodiments, a sample may be obtained from a human from the peripheral blood, peripheral venous blood, peripheral arterial blood, peripheral whole blood monocytes, peripheral mononuclear cell enriched monocytes, red blood cell lysate of whole peripheral blood, serum, plasma, tears, hair, sputum, bronchoalveoli, cerebrospinal fluid, pericardial fluid, pleural fluid, peritoneal fluid, synovial fluid, vaginal fluid, urethral fluid, pericarditis fluid, pleural effusion fluid, ascites fluid, saliva, sweat, tumor, lymph, lymphatic vessels, lymph node tissue, adenoid tissue, spleen, spleen cells, or cancer tissue.

A sample may comprise cells that are intact, cells that are dissociated, extracellular products that are cell-derived, or a combination thereof.

Extracellular products may be derived from cells that are dissociated from cells and extracellular in form and may comprise nucleic acids, proteins, lipids, carbohydrates, nanovesicles, microvesicles, glycated end-products, enzymes, chemical products of metabolism, chemical by-products of metabolism, cations, and anions.

### Diagnostic Methods

In some embodiments, a method of the current disclosure comprises selecting a therapeutic regimen. A method of selecting a therapeutic regimen may comprise: determining the presence or absence of cluster of differentiation 8 (CD8) protein, and at least one of: B-cell activating factor (BAFF) protein. B-cell activating factor receptor (BAFF-R) protein, and paired box Pax-5 (PAX5) protein in a cancer or a tumor of a subject or a cancer sample or a tumor sample of the subject; if CD8 protein and at least one of B-cell activating factor, B-cell activating factor-R, and PAX5 are present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof; if CD8 protein and at least one of B-cell activating factor, B-cell activating factor-R, and PAX5 is not present in the cancer or the tumor or the cancer sample or the tumor sample, selecting a therapeutic regimen comprising an anti-programmed cell death protein 1 agent, an anti-programmed death ligand 1 agent, an anti-programmed death ligand 2 agent, or a salt thereof or any combination thereof.

The method may further comprise determining the presence or absence of a number of proteins, or cell-surface proteins, wherein the proteins may be, but are not limited to, cluster of differentiation 19 (CD19) protein, cluster of differentiation 20 (CD20) protein, cluster of differentiation 138 (CD138) protein, or any combination thereof.

The method may be a method of treatment, wherein the treatment comprises: administering a therapeutically effective amount of an immunotherapeutic agent or a salt thereof, wherein the immunotherapeutic agent or a salt thereof inhibits a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof; wherein the immunotherapeutic agent is co-administered with a checkpoint inhibitor.

In some embodiments, the checkpoint inhibitor is selected from the group consisting of an anti-programmed cell death protein 1 agent, an anti-programmed death ligand 1 agent, an anti-programmed death ligand 2 agent, a salt of any of these, and any combination thereof. In some embodiments, the method comprises administering the anti-programmed cell death protein 1 agent of a salt thereof. In some embodiments, the anti-programmed cell death protein 1 agent or a salt thereof is selected from the group consisting of vemurafenib, erlotinib, docetaxel, nivolumab, pembrolizumab, and a salt of any of these. In some embodiments, the anti-programmed cell death protein 1 agent or a salt thereof is vemurafenib, erlotinib, or pembrolizumab. In some embodiments, the immunotherapeutic agent is tabalumab or a salt thereof. In some embodiments, the immunotherapeutic agent is belimumab or a salt thereof.

In some embodiments, a method of the current disclosure is a method of selecting a therapeutic regimen, comprising: determining a count of B cells. T cells, and plasma cells in a cancer or a tumor of a subject or a cancer sample or a tumor sample of the subject; if a count of T cells in the cancer or the tumor or the cancer sample or the tumor sample is greater than 500 cells/mm2 and a count of B cells is less than <150 cells/mm2, selecting a therapeutic regimen comprising an anti-programmed cell death protein 1 agent, an anti-programmed death ligand 1 agent, an anti-programmed death ligand 2 agent, or a salt thereof or any combination thereof; if a count of T cells in the cancer or the tumor or the cancer sample or the tumor sample is greater than 300 cells/mm2 and a count of B cells, a count of plasma cells, or a combination of B cells and plasma cells is greater than 100 cells/mm2, selecting a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof. In some embodiments, a method of the current disclosure comprises conveying a result via a communication medium.

In another aspect, the current disclosure provides a method of treating or maintaining remission of a cancer or a tumor in a subject, comprising: administering an anti-programmed cell death protein 1 agent, an anti-programmed death ligand 1 agent, an anti-programmed death ligand 2 agent, or a salt thereof or any combination thereof, if a percentage of T cells in a cancer sample or a tumor sample of a subject is greater than 10% and a percentage of B cells is less than 5%, or administering an anti-programmed cell death protein 1 agent, an anti-programmed death ligand 1 agent, an anti-programmed death ligand 2 agent, or a salt thereof or any combination thereof, and an immunotherapeutic agent or a salt thereof if a percentage of T cells in a cancer or a tumor sample of the subject is less than 10% and a percentage of B cells, a percentage of plasma cells, or a combination of the percentage of B cells and the percentage of plasma cells is greater than 10% in the cancer sample of the subject.

In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following is observed when compared to a count prior to treatment: a percentage of T cells, a percentage of B cells, and a percentage of plasma cells. In some embodiments, the cancer or the tumor is prostate cancer, testicular cancer, breast cancer, brain cancer, pancreatic cancer, colon cancer, thyroid cancer, stomach cancer, lung cancer, or ovarian cancer.

In another aspect, the current disclosure provides a method of assessing a likelihood of a subject having a cancer or a tumor exhibiting a clinically beneficial response to treatment, the method comprising: assessing a count of T cells, B cells, and plasma cells in a cancer or a tumor sample of a subject; and calculating, using a computer system, a probability of treatment responsiveness based on (1) a ratio between the count of T cells and a reference count of T cells, (2) a ratio between the count of B cells and a reference count of B cells, and/or (3) a ratio between the count of plasma cells and a reference count of plasma cells. In some embodiments, the method further comprises designating the subject as having a high probability of exhibiting a clinically beneficial response to treatment if the ratio between the count of B cells and the reference count of B cells is less than 1. In some embodiments, the method further comprises designating the subject as having a high probability of exhibiting a clinically beneficial response to treatment if the ratio between the count of plasma cells and the reference count of plasma cells is less than 1. In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following is observed when compared to a count prior to treatment: a count of T cells, a count of B cells, and a count of plasma cells.

In some embodiments, a method comprising surgery, radiation therapy, or administering a pharmaceutical agent, wherein the pharmaceutical agent is an immunotherapeutic agent, a chemotherapeutic agent, a targeted therapeutic agent, a hormonal therapy agent, cell-based therapy agent, radiation therapy agent, or any salt thereof, or any combination thereof.

In some embodiments, a method is described herein to determine a count of cluster of differentiation 8 (CD8) protein, a count of cluster of differentiation 19 (CD19) protein, a count of cluster of differentiation 20 protein (CD20), a count of cluster of differentiation 138 (CD138) protein, a count of B-cell activating factor protein, a count of B-cell activating factor receptor protein, a count of paired box Pax-5 (PAX5) protein, or any combination thereof, in a cancer or a tumor or a cancer sample or a tumor sample of a subject.

In some embodiments, at least one of a count of cluster of differentiation 19 (CD19) protein, a count of cluster of differentiation 20 protein (CD20), a count of cluster of differentiation 138 (CD138) protein, a count of B-cell activating factor protein, a count of B-cell activating factor receptor protein, a count of paired box Pax-5 (PAX5) protein, or any combination thereof, is present in a cancer or a tumor or a cancer sample or a tumor sample of a subject. In some embodiments, a least one of CD19, CD20, CD138, B-cell activating factor, B-cell activating factor-R, and PAX5 is individually present in a cancer or a tumor or a cancer sample or a tumor sample of a subject in an amount ranging from 1 protein to at least about 10,000,000 proteins. An amount of protein in a cancer or a tumor or a cancer sample or a tumor sample may range between 1 protein to 100,000,000 proteins.

In one aspect, the current disclosure provides a method of assessing a likelihood of a subject having a cancer or a tumor exhibiting a clinically beneficial response to treatment, the method comprising: assessing a count of T cells, B cells, and plasma cells in a cancer or a tumor sample of a subject; and calculating, using a computer system, a probability of treatment responsiveness based on (1) a ratio between the count of T cells and a reference count of T cells, (2) a ratio between the count of B cells and a reference count of B cells, and/or (3) a ratio between the count of plasma cells and a reference count of plasma cells.

In some embodiments, the method further comprises designating the subject as having a high probability of exhibiting a clinically beneficial response to treatment if the ratio between the count of B cells and the reference count of B cells is less than 1. In some embodiments, the method further comprises designating the subject as having a high probability of exhibiting a clinically beneficial response to treatment if the ratio between the count of plasma cells and the reference count of plasma cells is less than 1.

In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following is observed when compared to a count prior to treatment: a count of T cells, a count of B cells, and a count of plasma cells. In some embodiments, the cancer or the tumor is prostate cancer, testicular cancer, breast cancer, brain cancer, pancreatic cancer, colon cancer, thyroid cancer, stomach cancer, lung cancer, or ovarian cancer. In some embodiments, the method further comprises surgery, radiation therapy, or administering a pharmaceutical agent, wherein the pharmaceutical agent is an immunotherapeutic agent, a chemotherapeutic agent, a targeted therapeutic agent, a hormonal therapy agent, cell-based therapy agent, radiation therapy agent, or any salt thereof, or any combination thereof.

In another aspect, the current disclosure provides a method of treating a cancer or a tumor in a subject, comprising: administering an anti-programmed cell death protein 1 agent, an anti-programmed death ligand 1 agent, an anti-programmed death ligand 2 agent, or a salt thereof or any combination thereof, if a cancer or a tumor sample of a subject contains at least one cell contains a cluster of differentiation 8 (CD8) protein and no cell contains a cluster of differentiation 19 (CD19) protein or a cluster of differentiation 138 (CD138) protein; or administering an anti-programmed cell death protein 1 agent, an anti-programmed death ligand 1 agent, an anti-programmed death ligand 2 agent, or a salt thereof or any combination thereof, and an immunotherapeutic agent or a salt thereof if a cancer or a tumor sample of a subject contains at least one cell contains a CD8 protein and at least one cell contains a CD19 or CD138 protein.

In some embodiments, after the administration of the anti-programmed cell death protein 1 agent, the anti-programmed death ligand 1 agent, the anti-programmed death ligand 2 agent, or a salt thereof or any combination thereof, a decrease in at least one of the following is observed when compared to a count prior to treatment: a count of T cells, a count of B cells, and a count of plasma cells. In some embodiments, after the administration of the anti-programmed cell death protein 1 agent, the anti-programmed death ligand 1 agent, the anti-programmed death ligand 2 agent, or a salt thereof or any combination thereof, and an immunotherapeutic agent or a salt thereof, a decrease in at least one of the following is observed when compared to a count prior to treatment: a count of T cells, a count of B cells, and a count of plasma cells.

In some embodiments, a decrease in a count of T cells, a decrease in a count of B cells, a decrease in a count of plasma cells, or any combination thereof, is observed. In some embodiments, the method is a method of treating a cancer or a tumor in the subject. In some embodiments, the cancer or the tumor is prostate cancer, testicular cancer, breast cancer, brain cancer, pancreatic cancer, colon cancer, thyroid cancer, stomach cancer, lung cancer, or ovarian cancer.

In one aspect, the current disclosure provides a method of treating a cancer or a tumor in a subject, comprising administering a therapeutically effective amount of a pharmaceutical agent and an immunotherapeutic agent. In some embodiments, the presence of the CD8 protein, the CD19 protein, the CD20 protein, the CD139 protein, the B-cell activating factor protein, the B-cell activating factor-R protein, and the PAX5 protein is determined by staining, imaging after staining, microscopy, or any combination thereof. In some embodiments, the staining comprises binding of an antibody to the protein. In some embodiments, the staining further comprising binding of a second antibody to a first antibody, wherein the second antibody contains a fluorescence marker.

In some embodiments, the method is repeated after administration of the therapeutic. In some embodiments, the cancer sample or the tumor sample comprises at least one cell comprising a CD8 protein and at least one cell comprising a CD19 protein, wherein the CD8 protein and the CD19 protein have a distance apart of at most 100 microns. In some embodiments, the cancer sample or the tumor sample comprises at least one cell comprising a CD8 protein and at least one cell comprising a CD19 protein, wherein the CD8 protein-containing cell and the CD19 protein-containing cell have a distance apart of at most 100 microns.

In some embodiments, a method described herein comprises determining the number of aggregates of cells within a tumor sample or cancer sample of a subject. An aggregate of cells may be a cluster of cells. An aggregate may comprise at least about 1, 2, 3, 4, 5, 10, 15, 20, 30, 40, 50, 60, 70, 80, 90, 100, 200, 300, 400, 500, 600, 700, 800, 900, 1000, 10,000, 100,000, 1,000,000 or more cells. In some embodiments, an aggregate of cells may comprise about 1 cell to about 100 cells, about 10 cells to about 50 cells, or about 20 cells to about 40 cells.

In some embodiments, an aggregate of cells may be an aggregate of T-cells. In some embodiments, an aggregate of cells may be an aggregate of B-cells. In some embodiments, an aggregate of cells may comprise T-cells, B-cells, or a combination thereof. In some embodiments, a type of cell, or a cell type, may refer to a B-cell. In some embodiments, a type of cell, or a cell type, may refer to a T-cell.

In some embodiments, an aggregate comprises a cell of hematopoetic lineage. In some embodiments, an aggregate may comprise myeloid-derived cells, monocytes, macrophages, dendritic cells, mast cells, granulocytes, neutrophils, eosinophils, basophils, megakaryocytes, lymphoid-derived cells, NK-cells, T-cells, CD4, CD8, Gamma/delta T-cells, B-cells, plasma cells, or a combination thereof.

In some embodiments, the number of aggregates may be determined by: immunoassays, polymerase chain reaction, sequencing, including next generation sequencing, or flow cytometry. In some embodiments, the number of aggregates may be determined by: immunohistochemistry-chromogenic based, quantitative-based on the cellular level, quantitative-based on the single pixel level immunohistochemistry-fluorescence-based, quantitative-based on the cellular level, quantitative-based on the single pixel level, RNA in situ hybridization-chromogenic based, quantitative-based on the dot level, quantitative-based on the single pixel level, RNA in situ hybridization-fluorescent based, quantitative-based on the dot level, quantitative-based on the single pixel level or a combination thereof. Assays may be performed in single or multiplex formats. An aggregate may be detected using a nuclear counterstain.

Assay reagents that may be used include: antibody-based reagents, primary antibody+secondary antibody, detectable label: enzymatic, chromogenic-based, detectable label: enzymatic, fluorescent-based, primary antibody+oligonucleotide, detectable label: complementary hybridization+PCR amplification, detectable label: complementary hybridization without PCR amplification, RNA-based, DNA-based reagents.

Proximity of cells may be defined as the closest distance between an edge of a first cell and an edge of a second cell. Proximity of cells may be defined as at least about 0.01 micrometer (μm), 0.1, 0.5, 1, 2, 3, 4, 5, 6, 7, 8, 9, 10, or more in distance. In some embodiments, a first cell may be proximal in distance to a second cell, wherein the distance is about 0.01 μm to about 10 μm, about 0.1 μm to about 7 μm, or about 1 μm to about 5 μm. In some embodiments, a first cell may be proximal in distance to a second cell, wherein the distance between the two cells is less than about 1 μm, 0.1 μm, or 0.01 μm. In some cases, the distance between the two cells may be small such that the cell walls are in contact with each other.

In some embodiments, a tumor or cancer may be classified as a Type I resistant tumor or cancer or a Type II resistant tumor or cancer. A Type I resistant tumor or cancer may possess low counts of CD8 when imaged both before and during anti-PDL1 single agent treatment. A Type II resistant tumor or cancer may possess high counts of CD8 when imaged both before and during anti-PDL1 single agent treatment.

If a tumor or cancer is classified as a Type II resistant tumor or cancer, then a single agent treatment may not be as therapeutically effective as desired. In some embodiments, if a tumor or cancer is classified as a Type II resistant tumor or cancer, then a combination therapy, or a therapeutic regimen comprising more than one active agent, may be administered. In some embodiments, a Type II resistant tumor or cancer may be administered a therapeutic regimen comprising an anti-programmed cell death protein 1 agent, an anti-programmed death ligand 1 agent, an anti-programmed death ligand 2 agent, or a salt thereof or any combination thereof, and an immunotherapeutic agent or a salt thereof.

### Pharmaceutical Compositions

Provided herein, in certain embodiments, are compositions comprising a therapeutically effective amount of an immunotherapeutic agent and a checkpoint inhibitor.

In some embodiments, an inhibitor may refer to an agent or a therapy that may be an agonist, an antagonist, a partial agonist, a partial antagonist, or any combination thereof.

In some embodiments, an immunotherapeutic agent or a salt thereof inhibits a biological cascade selected from the group consisting of a B-cell activating factor receptor cascade, a transmembrane activator and calcium-modulator and cyclophilin ligand interactor receptor cascade, a B-cell maturation antigen receptor cascade, and any combination thereof. In some embodiments, an immunotherapeutic agent or a salt thereof.

A checkpoint inhibitor may be an anti-programmed cell death protein 1 agent, an anti-programmed death ligand 1 agent, an anti-programmed death ligand 2 agent, a salt of any of these, and any combination thereof.

An anti-programmed cell death protein 1 (PD-1) agent may be an agent that inhibits or at least partially inhibits the binding of the PD-1 cell surface receptor to a ligand, wherein the ligand may be anti-programmed death ligand 1 (PD-L1) or anti-programmed death ligand 2 (PD-L2). While not wishing to be bound by theory, inhibition of the binding of the PD-1 receptor to a ligand may activate an immune response.

An immunotherapeutic agent may be an agent that at least partially blocks a B-cell activating factor (BAFF) receptor cascade.

In some embodiments, the immunotherapeutic agent or salt thereof inhibits a B-cell activating factor receptor (BAFF) cascade, a transmembrane activator and calcium-modulator and cyclophilin ligand interactor receptor (TACI) cascade, a B-cell maturation antigen receptor (BCMA) cascade, and any combination thereof.

In some embodiments, an immunotherapeutic agent or a salt thereof at least partially prevents binding of a ligand to a B-cell activating factor receptor, is a partial antagonist of a B-cell activating factor receptor, is a partial agonist of a B-cell activating factor receptor, is a competitive antagonist of a B-cell activating factor receptor, is a non-competitive antagonist of a B-cell activating factor receptor, or any combination thereof. In some embodiments, immunotherapeutic agent or a salt thereof at least partially prevents ligand binding to a transmembrane activator and calcium-modulator and cyclophilin ligand interactor receptor, is a partial antagonist of a transmembrane activator and calcium-modulator and cyclophilin ligand interactor receptor, is a partial agonist of a transmembrane activator and calcium-modulator and cyclophilin ligand interactor receptor, or any combination thereof. In some embodiments, an immunotherapeutic agent or a salt thereof at least partially prevents ligand binding to a B-cell maturation antigen receptor, is a partial antagonist of a B-cell maturation antigen receptor, is a partial agonist of a B-cell maturation antigen receptor, or any combination thereof.

An immunotherapeutic agent may be a small molecule, an antibody, a partial antibody, or any combination thereof. In some embodiments, an immunotherapeutic agent is an antibody or a salt thereof. The immunotherapeutic agent may be a human antibody, a humanized monoclonal antibody, or a salt thereof.

In some embodiments, the immunotherapeutic agent or a salt thereof is ardenermin, briobacept, blisibimod, belimumab, a transmembrane activator and calcium-modulator and cyclophilin ligand interactorcept, MEDI-0700, NOV-5, rGel/BLyS, tabalumab, a salt of any of these, and any combination thereof. In some embodiments, an immunotherapeutic agent or a salt thereof is selected from the group consisting of MEDI-700, NOV-5, rGel/BLyS, a salt of any of these, and any combination thereof. In some embodiments, the immunotherapeutic agent is tabalumab or a salt thereof. In some embodiments, the immunotherapeutic agent is blisibimod or a salt thereof. In some embodiments, the immunotherapeutic agent is belimumab or a salt thereof. In some embodiments, a BAFF/BAFF-R inhibitor may be combined with avelumab. In some embodiments, a BAFF/BAFF-R inhibitor may be combined with pembrolizumab.

In some embodiments, a PD-L1 inhibitor may be avelumab. In some embodiments, a PD-1 inhibitor may be pembrolizumab. In some embodiments, a PD-L1 inhibitor may be atezoluzumab.

An immunotherapeutic agent may be an antibody. An immunotherapeutic agent may be a human monoclonal antibody. An immunotherapeutic agent may be a humanized monoclonal antibody, chimeric antibody, or an isolated or purified version of any of the above. An immunotherapeutic agent may be tabalumab or a fragment or a salt thereof. Any compound, molecule, protein, antibody, antibody fragment, or salt of any thereof, described herein can be isolated and purified.

An immunotherapeutic agent can be a checkpoint inhibitor, an immune agonist, a vaccine, a virus, or a T-cell.

Provided herein, in certain embodiments, are compositions comprising a therapeutically effective amount of a pharmaceutical agent and an immunotherapeutic agent. Provided herein, in certain embodiments, are compositions comprising a therapeutically effective amount of a pharmaceutical agent and an immunotherapeutic agent, wherein the immunotherapeutic agent is tabalumab or a fragment or a salt thereof.

In certain embodiments are compositions comprising a therapeutically effective amount of a pharmaceutical agent and an immunotherapeutic agent, wherein the immunotherapeutic agent is briobacept, rGel/BLyS, BLyS radiolabeled, Xencor, NOV-5, ardenermin, anti-BR3, blisibimod, belimumab, IL-17-bispecific antibody, anti-BLyS/APRIL antibody fusion protein, or a fragment or a salt thereof.

In certain embodiments are compositions comprising a therapeutically effective amount of a pharmaceutical agent and an immunotherapeutic agent, wherein the immunotherapeutic agent is florbetapir, solanezumab, scyllo-inositol, gantenerumab, flutafuranol F18, cromolyn sodium+ibuprofen. AZTherapics, crenezumab, CAD-106, aducanumab, UB-311, GSK-933776A, BAN-2401, ACI-24, VM-100, SAR-228810, NGP-555, MRZ-99030, MEDI-1814, LY-3002813, Lu-AF-20513, HSRx 888+donepezil hydrochloride, Exebryl-1, bisnorcymserine, anti-amyloid beta antibody, Kyowa Hakko Kirin, ALZ-801, Affitope AD-03, AAB-003, (+)-phenserine, TRV-101, RV-03, P-8, NPT440-1, MRK-560, KAL-ABP, IN-NO1-OX2, IN-N01, CB-301, C12, Pharma Bio, CT-01344, BAN-2502, AZP-2006, anti-beta amyloid, Aerie, anti-amyloid beta programme, anti-amyloid beta antibody-2, anti-amyloid beta antibody, ALZT-Patch, ALZ-201, AGT-160, ADx, ACU-193, ACI-812, ACI-260, ZT-331, ACC-001, tramiprosate, SIB-1281, ponezumab, PF-4382923, GT3001, GT2501, GT2342, bapineuzumab, AN-1792, ACU-244, V-950, U-101033E, CHF-5022, ScV-2-0401, SEN-1576, SEN-1500, SEN-1269, SEN-1176, RV-02, RV-01, RS-0406, PX-106, PTTI-L07665, PTT-48579, PTI-3001, PTI-2001, PTI-00703, protease nexin-1, SIBIA, phenserine(−)-eseroline phenylcarbamate(−)-phenserine, Pharmaprojects No. 5550, Pharmaprojects No. 4246, DP-74, NU-700, NP-0361, MPI-442690, MPI-423948MPI-127585, MK-3328, MER-5101, KMS-88009KMS-88016, INM-176, ESBA-212, EDN-OL1, DP-68, DLX-212, DBT-1339, Cymserine, N1-phenethylnorcymserine, CLR-01, CaprospinolSP-233, BAN-2203AD-2203, AZD-2995, AZD-2184, ARC-031, amyloid inhibiting peptides, amyloid beta modulator, AMDL receptor antagonist, ALZT-OP2, SPI-008SPI-0090, SPI-019, AGT-3100, AGT-100, Affitope AD-02, Affitope AD-01, ACU-CD0061, ACU-5A5, ACU-347, ACU-193, ABP-102, DRM-106, or a fragment or a salt thereof.

A pharmaceutical agent may be an immunotherapeutic agent, a chemotherapeutic agent, a targeted therapeutic agent, a hormonal therapy agent, cell-based therapy agent, or radiation therapy agent.

In some embodiments, a composition may comprise a pharmaceutical agent and an immunotherapeutic agent, wherein the immunotherapeutic agent is a B-cell activating factor inhibitor and at least partially blocks the BAFF receptor cascade.

Pharmaceutical compositions may be formulated using one or more physiologically acceptable carriers including excipients and auxiliaries which facilitate processing of the pharmaceutical composition into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa., Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

The compositions and methods of the present disclosure may be utilized to treat an individual in need thereof. In certain embodiments, the individual is a mammal such as a human, or a non-human mammal. In some embodiments, a subject is a human. In certain embodiments, the human is an adult male, an adult female, a female child, or a male child. When administered to an animal, such as a human, the composition or the pharmaceutical composition, can be administered as a pharmaceutical composition comprising, for example, a therapeutically effective amount of an anti-programmed cell death protein 1 agent and an immunotherapeutic agent and a pharmaceutically acceptable carrier or excipient. Pharmaceutically acceptable carriers can include, for example, aqueous solutions such as water or physiologically buffered saline or other solvents or vehicles such as glycols, glycerol, oils such as olive oil, or injectable organic esters. In an embodiment, when such pharmaceutical compositions are for human administration, particularly for invasive routes of administration, e.g., routes, such as injection or implantation, that circumvent transport or diffusion through an epithelial barrier, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule, granule, lyophile for reconstitution, powder, solution, syrup, suppository, injection or the like. The composition can also be present in a transdermal delivery system. e.g., a skin patch. The composition can also be present in a solution suitable for topical administration, such as an eye drop.

A pharmaceutically acceptable excipient can contain physiologically acceptable agents that act, for example, to stabilize, increase solubility or to increase the absorption of a composition such as a therapeutically effective amount of an anti-programmed cell death protein 1 agent and an immunotherapeutic agent. Such physiologically acceptable agents caninclude, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable excipient, including a physiologically acceptable agent, depends, for example, on the route of administration of the composition. The preparation or pharmaceutical composition can be a self emulsifying drug delivery system or a self microemulsifying drug delivery system. The pharmaceutical composition (preparation) also can be a liposome or other polymer matrix, which can have incorporated therein, for example, a therapeutically effective amount of an anti-programmed cell death protein 1 agent and an immunotherapeutic agent. Liposomes, for example, which comprise phospholipids or other lipids, are nontoxic, physiologically acceptable and metabolizable carriers that are relatively simple to make and administer.

In some embodiments, the immunotherapeutic agent or a salt thereof is administered in a pharmaceutical composition.

In one aspect, the current disclosure provides a composition comprising an anti-programmed cell death protein 1 agent, an anti-programmed death ligand 1 agent, an anti-programmed death ligand 2 agent, or a salt of any one thereof, or any combination thereof, and an immunotherapeutic agent or a salt thereof.

In some embodiments, the composition is a pharmaceutical composition. In some embodiments, the pharmaceutical composition is in unit dose form. In some embodiments, the immunotherapeutic agent or a salt thereof is an antibody or a salt thereof. In some embodiments, the immunotherapeutic agent or a salt thereof is a human or humanized monoclonal antibody or a salt thereof. In some embodiments, the immunotherapeutic agent or a salt thereof is selected from the group consisting of ardenermin, briobacept, blisibimod, belimumab, atransmembrane activator and calcium-modulator and cyclophilin ligand interactorcept, MEDI-0700, NOV-5, rGel/BLyS, tabalumab, and a salt of any of these. In some embodiments, the immunotherapeutic agent or a salt thereof is tabalumab or a salt thereof. In some embodiments, a composition further comprising a pharmaceutically acceptable excipient.

In some embodiments, the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof are independently administered in an amount from about 0.1 mg to about 10 g. In some embodiments, the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof are independently 0.001% to 99% by weight of the composition, the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof are independently administered in an amount of about 0.1 mg to about 100 mg per kg body weight.

In some embodiments, the composition is in a form of a tablet, a capsule, a gel, or a liquid formulation.

In one aspect, the present disclosure provides for a kit comprising a composition of any one of the preceding claims. In some embodiments, the kit contains instructions for use.

In one aspect, the present disclosure provides a method of making a kit disclosed herein, comprising contacting or combining the anti-programmed cell death protein 1 agent, the anti-programmed death ligand 1 agent, the anti-programmed death ligand 2 agent, or a salt of any one thereof, or any combination thereof, and the immunotherapeutic agent or a salt thereof. In some embodiments, the composition is in a form of a tablet, a capsule, a gel, or a liquid formulation.

In one aspect, the present disclosure provides a method of making a composition of any one of the preceding claims, comprising contacting or combining the anti-programmed cell death protein 1 agent, the anti-programmed death ligand 1 agent, the anti-programmed death ligand 2 agent, or a salt thereof or any combination thereof, and the immunotherapeutic agent or a salt thereof.

### Routes of Administration

A pharmaceutical composition can be administered to a subject by any of a number of routes of administration including, for example, orally, for example, drenches as in aqueous or non-aqueous solutions or suspensions, tablets, capsules, including sprinkle capsules and gelatin capsules, boluses, powders, granules, pastes for application to the tongue; absorption through the oral mucosa. e.g., sublingually; anally, rectally or vaginally, for example, as a pessary, cream or foam; parenterally, including intramuscularly, intravenously, subcutaneously or intrathecally as, for example, a sterile solution or suspension; nasally: intraperitoneally; subcutaneously; transdermally, for example, as a patch applied to the skin; and topically, for example, as a cream, ointment or spray applied to the skin, or as an eye drop. The pharmaceutical composition may also be formulated for inhalation. In certain embodiments, a therapeutically effective amount of an anti-programmed cell death protein 1 agent and an immunotherapeutic agent may be simply dissolved or suspended in sterile water. In some embodiments, a composition is administered directly to a cancer or a tumor. In some embodiments, administration of a composition is to a different location of the body than where the cancer or tumor is detected or present.

A pharmaceutical composition may be a sterile aqueous or non-aqueous solution, suspension or emulsion, e.g., a microemulsion. The excipients described herein are examples and are in no way limiting. An effective amount or therapeutically effective amount can refer to an amount of the one or more pharmaceutical compositions administered to a subject, either as a single dose or as part of a series of doses, which is effective to produce a desired therapeutic effect.

Subjects may generally be monitored for therapeutic effectiveness using assays and diagnostic methods suitable for the condition being treated. Pharmacokinetics of a therapeutically effective amount of an anti-programmed cell death protein 1 agent and an immunotherapeutic agent, or one or more metabolites thereof, that is administered to a subject may be monitored by determining the level of the pharmaceutical composition or metabolite in a biological fluid, for example, in the blood, blood fraction, e.g., serum, and/or in the urine, and/or other biological sample or biological tissue from the subject.

The dose of a therapeutically effective amount of an anti-programmed cell death protein 1 agent and an immunotherapeutic agent described herein for treating a cancer may depend upon the subject's condition, that is, stage of the disease, severity of symptoms caused by the disease, general health status, as well as age, gender, and weight, and other factors apparent to a person skilled in the medical art. In some embodiments, a subject has been previously diagnosed with a cancer or a tumor. In some embodiments, a subject has previously undergone treatment for a cancer or a tumor. In some embodiments, the subject is currently undergoing treatment for a cancer or a tumor. In some embodiments, the current treatment of the subject is less than therapeutically effective. In some embodiments, the subject was previously treated with a checkpoint inhibitor, and the cancer or the tumor was at least partially refractive. In some embodiments, the cancer or tumor may be considered partially refractive if the mass or volume of the cancer or tumor continues to grow at a constant rate, or if the mass or volume of the cancer or tumor does not substantially change or decrease.

Pharmaceutical compositions may be administered in a manner appropriate to the disease to be treated as determined by persons skilled in the medical arts. In addition to the factors described herein and above related to use of pharmaceutical composition for treating a cancer, suitable duration and frequency of administration of the pharmaceutical composition may also be determined or adjusted by such factors such as the condition of the subject, the type and severity of the subject's disease, the particular form of the active ingredient, and the method of administration. Optimal doses of an agent may generally be determined using experimental models and/or clinical trials. The optimal dose may depend upon the body mass, weight, or blood volume of the subject. Design and execution of pre-clinical and clinical studies for a pharmaceutical composition, including when administered for prophylactic benefit, described herein are within the skill of a person skilled in the relevant art.

When two or more pharmaceutical compositions are administered to treat a cancer, the optimal dose of each pharmaceutical composition may be different, such as less than when either agent is administered alone as a single agent therapy. In certain particular embodiments, two pharmaceutical compositions in combination may act synergistically, and either agent may be used in a lesser amount than if administered alone. In certain particular embodiments, administration of a checkpoint inhibitor and an immunotherapeutic agent may act synergistically or more than additively, when compared to either agent administered alone. In particular embodiments, a composition comprising a checkpoint inhibitor and an immunotherapeutic agent may produce a more than additive effect on a tumor or a cancer. The synergy of a combination of the current disclosure may be at least 1%., 2%, 3%, 5%, 10%, 20%., 30%, 40%, 50%, 60%, 70%., 80%, 90%, or more greater than the additive effect.

In some embodiments, a composition of the disclosure comprising an anti-programmed cell death protein 1 agent and an immunotherapeutic agent is more effective as compared to the corresponding additive effect of the anti-programmed cell death protein 1 agent and an immunotherapeutic agent. A composition of the disclosure can be at least 15% more effective, at least 20% more effective, at least 25% more effective, at least 30% more effective, at least 35% more effective, at least 40% more effective, at least 45% more effective, at least 50% more effective, at least 55% more effective, at least 60% more effective, at least 65% more effective, at least 70% more effective, at least 75% more effective, at least 80% more effective, at least 85% more effective, at least 95% more effective, or at least 100% more effective than the effect of a monotherapy comprising an anti-programmed cell death protein 1 agent, and/or the effect of a monotherapy comprising an immunotherapeutic agent.

A composition of the disclosure can be at least 1% more effective, at least 5% more effective, at least 10% more effective, at least 15% more effective, at least 20% more effective, at least 25% more effective, at least 30% more effective, at least 35% more effective, at least 40% more effective, at least 45% more effective, at least 50% more effective, at least 55% more effective, at least 60% more effective, at least 65% more effective, at least 70% more effective, at least 75% more effective, at least 80% more effective, at least 85% more effective, at least 95% more effective, or at least 100% more effective than the corresponding additive effect of the anti-programmed cell death protein 1 agent and an immunotherapeutic agent.

An amount of a pharmaceutical composition or active therein that may be administered per day may be, for example, between about 0.01 mg/kg and 100 mg/kg, e.g., between about 0.1 to 1 mg/kg, between about 1 to 10 mg/kg, between about 10-50 mg/kg, between about 50-100 mg/kg body weight. In other embodiments, the amount of a pharmaceutical composition that may be administered per day may be between about 0.01 mg/kg and 1000 mg/kg, between about 100-500 mg/kg, or between about 500-1000 mg/kg body weight. The optimal dose, per day or per course of treatment, may be different for the cancer or tumor to be treated and may also vary with the administrative route and therapeutic regimen.

Pharmaceutical compositions comprising a pharmaceutical composition can be formulated in a manner appropriate for the delivery method by using techniques routinely practiced in the art. The composition may be in the form of a solid, e.g., tablet, capsule, semi-solid, e.g., gel, liquid, or gas, e.g., aerosol. In other embodiments, the pharmaceutical composition is administered as a bolus infusion.

Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used. The type of excipient selected may be based on the mode of administration, as well as the chemical composition of the pharmaceutical composition. Alternatively, compositions described herein may be formulated as a lyophilizate. A composition described herein may be lyophilized or otherwise formulated as a lyophilized product using one or more appropriate excipient solutions for solubilizing and/or diluting the pharmaceutical composition of the composition upon administration. In other embodiments, the pharmaceutical composition may be encapsulated within liposomes. Pharmaceutical compositions may be formulated for any appropriate manner of administration described herein and in the art.

In certain particular embodiments, a pharmaceutical composition, which may be combined with at least one pharmaceutically acceptable excipient to form a pharmaceutical composition, is administered directly to the target tissue or organ comprising tumor cells that contribute to manifestation of the disease or disorder.

A pharmaceutical composition, e.g., for oral administration or for injection, infusion, subcutaneous delivery, intramuscular delivery, intraperitoneal delivery or other method, may be in the form of a liquid. A liquid pharmaceutical composition may include, for example, one or more of the following: a sterile diluent such as water, saline solution, physiological saline solution, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline can be used, and an injectable pharmaceutical composition can be sterile. In another embodiment, for treatment of an ophthalmological condition or disease, a liquid pharmaceutical composition may be applied to the eye in the form of eye drops. A liquid pharmaceutical composition may be delivered orally.

For oral formulations, at least one of the pharmaceutical compositions described herein can be used alone or in combination with appropriate additives to make tablets, powders, granules or capsules, and if desired, with diluents, buffering agents, moistening agents, preservatives, coloring agents, and flavoring agents. The pharmaceutical compositions may be formulated with a buffering agent to provide for protection of the pharmaceutical composition from low pH of the gastric environment and/or an enteric coating. A pharmaceutical composition included in a pharmaceutical composition may be formulated for oral delivery with a flavoring agent, e.g., in a liquid, solid or semi-solid formulation and/or with an enteric coating.

A pharmaceutical composition comprising any one of the pharmaceutical compositions described herein may be formulated for sustained release, slow release, timed release, or controlled release. Such compositions may generally be prepared and administered by, for example, oral, rectal, intradermal, or subcutaneous implantation, or by implantation at the desired target site. Sustained-release formulations may contain the pharmaceutical composition dispersed in a carrier matrix and/or contained within a reservoir surrounded by a rate controlling membrane. Excipients for use within such formulations are biocompatible, and may also be biodegradable. The amount of pharmaceutical composition contained within a sustained release formulation depends upon the site of implantation, the rate and expected duration of release, and the nature of the condition, disease or disorder to be treated or prevented.

In certain embodiments, the pharmaceutical compositions comprising a pharmaceutical composition are formulated for transdermal, intradermal, or topical administration. The compositions can be administered using a syringe, bandage, transdermal patch, insert, or syringe-like applicator, as a powder/talc or other solid, liquid, spray, aerosol, ointment, foam, cream, gel, paste. The active compositions can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

Pharmaceutical compositions can be formulated as emulsions for topical application. In some embodiments, the pharmaceutical composition described herein can be formulated as in inhalant. Inhaled methods can deliver medication directly to the airway. In some embodiments, the pharmaceutical composition can be formulated with oleaginous bases or ointments to form a semisolid composition with a desired shape.

Controlled or sustained release transdermal or topical formulations can be achieved by the addition of time-release additives, such as polymeric structures, and matrices. For example, the compositions may be administered through use of hot-melt extrusion articles, such as bioadhesive hot-melt extruded film. The formulation can comprise a cross-linked polycarboxylic acid polymer formulation. A cross-linking agent may be present in an amount that provides adequate adhesion to allow the system to remain attached to target epithelial or endothelial cell surfaces for a sufficient time to allow the desired release of the pharmaceutical composition.

A polymer formulation can also be utilized to provide controlled or sustained release. Bioadhesive polymers may be used. By way of example, a sustained-release gel and the pharmaceutical composition may be incorporated in a polymeric matrix, such as a hydrophobic polymer matrix. Examples of a polymeric matrix include a microparticle. The microparticles can be microspheres, and the core may be of a different material than the polymeric shell. Alternatively, the polymer may be cast as a thin slab or film, a powder produced by grinding or other standard techniques, or a gel such as a hydrogel. The polymer can also be in the form of a coating or part of a bandage, stent, catheter, vascular graft, or other device to facilitate delivery of the pharmaceutical composition. The matrices can be formed by solvent evaporation, spray drying, or solvent extraction.

Kits with unit doses of one or more of the agents described herein, usually in oral or injectable doses, are provided. Such kits may include a container containing the unit dose, an informational package insert describing the use and attendant benefits of the drugs in treating a tumor or cancer, instructions for use, and optionally an appliance or device for delivery of the composition.

### Dosing and Treatment Regimens

The pharmaceutical compositions described herein can be used in the preparation of medicaments for the prevention or treatment of a cancer. In addition, a method for treating any of the diseases or conditions described herein in a subject in need of such treatment, involves administration of pharmaceutical compositions containing at least a therapeutically effective amount of an anti-programmed cell death protein 1 agent and an immunotherapeutic agent, or a pharmaceutically acceptable salt, pharmaceutically acceptable prodrug, or pharmaceutically acceptable solvate thereof, in therapeutically effective amounts to the subject.

In some embodiments, the present disclosure provides a method for treating any of the diseases or conditions described herein in a subject in need of such treatment, wherein the treatment involves administration of pharmaceutical composition containing at least a therapeutically effective amount of an anti-programmed cell death protein 1 agent and an immunotherapeutic agent, wherein the immunotherapeutic agent is tabalumab or a fragment or a salt thereof.

A count of B cells, or a component thereof, or plasma cells, or a component thereof, may be observed in a sample of whole blood, blood serum, blood plasma, tumor, saliva, sweat, or urine of a subject. A specific treatment method for each individual subject may be determined based on a count of B cells or plasma cells or T cells, or any combination thereof.

In some embodiments, the present disclosure provides a method of administering an anti-programmed cell death protein 1 agent or a salt thereof or an anti-PD-L1 agent or a salt thereof if a count of T cells in a cancer or a tumor sample of a subject is greater than 10 cells/mm², 100 cells/mm², 200 cells/mm², 300 cells/mm², 400 cells/mm², 500 cells/mm², 600 cells/mm², 700 cells/mm², 800 cells/mm², 900 cells/mm², or 1000 cells/mm², and a count of B cells is less than 1000 cells/mm², 900 cells/mm², 800 cells/mm², 700 cells/mm², 600 cells/mm², 500 cells/mm², 400 cells/mm², 300 cells/mm², 200 cells/mm², 150 cells/mm², 100 cells/mm², 90 cells/mm², or 75 cells/mm².

In some embodiments, the present disclosure provides a method of administering an anti-programmed cell death protein 1 agent or a salt thereof or an anti-PD-L1 agent or a salt thereof, and an immunotherapeutic agent at a salt thereof if a count of T cells in a cancer or a tumor sample of a subject is greater than 10 cells/mm², 100 cells/mm², 200 cells/mm², 300 cells/mm², 400 cells/mm², 500 cells/mm², 600 cells/mm², 700 cells/mm², 800 cells/mm², 900 cells/mm², or 1000 cells/mm² and a count of B cells, a count of plasma cells, or a combination of B cells and plasma cells is greater than 10 cells/mm², 100 cells/mm², 200 cells/mm², 300 cells/mm², 400 cells/mm², 500 cells/mm², 600 cells/mm², 700 cells/mm², 800 cells/mm², 900 cells/mm², or 1000 cells/mm² in a cancer or a tumor sample of a subject.

In some embodiments, the present disclosure provides a method of administering an anti-programmed cell death protein 1 agent or a salt thereof or an anti-PD-L1 agent or a salt thereof if a percentage of T cells in a cancer sample or a tumor sample of a subject is greater than 5%, 10%, 15%., 20%, 30%, 40%, 50%, 60%, 70%, 80%, 90%, or more, and a percentage of B cells is less than 50%, 40%, 30%, 20%, 15%, 10%, 5%, or less.

In some embodiments, the present disclosure provides a method of administering an anti-programmed cell death protein 1 agent or a salt thereof or an anti-PD-L1 agent or a salt thereof, and an immunotherapeutic agent or a salt thereof if a percentage of T cells in a cancer or a tumor sample of a subject is less than 50%, 40%, 30%, 20%, 15%, 10%, 5%, or less, and a percentage of B cells, a percentage of plasma cells, or a combination of a percentage of B cells and a percentage of plasma cells is greater than 5%, 10%, 15%, 20%, 30%, 40%, 50%, 60%, 70%, 80%, or 90% in a cancer sample of a subject.

The compositions containing the pharmaceutical compositions described herein can be administered for prophylactic and/or therapeutic treatments. In therapeutic applications, the compositions are administered to a subject already suffering from a disease or condition, in an amount sufficient to cure or at least partially arrest the symptoms of the disease or condition. Amounts effective for this use may depend on the severity and course of the disease or condition, previous therapy, the subject's health status, weight, and response to the drugs, and the judgment of the treating physician.

In the case wherein the subject's condition does not improve, upon the doctor's discretion the administration of the pharmaceutical composition may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the administration of the pharmaceutical composition may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% relative to a dosage administered before or after the drug holiday.

In some cases, the administration of the pharmaceutical composition may be given indefinitely.

In the cases, a subject may exhibit a desired response to an anti-programmed cell death protein 1 therapy. In some cases, a subject may exhibit a partial desired response to an anti-programmed cell death protein 1 therapy. In some cases, a subject may become resistant to a monotherapy. A subject may become resistant, or show signs of resistance, after 1 day, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, 400 days, 500 days, 600 days, 700 days, 800 days, 900 days, or more after treatment of a monotherapy.

Once improvement of the subject's conditions has occurred, a maintenance dose is administered if necessary. Subsequently, the dosage or the frequency of administration, or both, can be reduced, as a function of the symptoms, to a level at which the improved disease, disorder or condition is retained. Subjects can, however, require intermittent treatment on a long-term basis upon any recurrence of symptoms.

The amount of a given agent that may correspond to such an amount may vary depending upon factors such as the particular pharmaceutical composition, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment may typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more pharmaceutical compositions. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The single dosages appropriate for the pharmaceutical compositions described herein are from about 0.01 mg/kg to about 20 mg/kg. In one embodiment, the single dosage of a checkpoint inhibitor is from about 0.1 mg/kg to about 10 mg/kg. In one embodiment, the single dosage of an anti-programmed cell death protein 1 agent, an anti-programmed death ligand 1 agent, an anti-programmed death ligand 2 agent, a salt of any one thereof, or any combination thereof, is from about 0.1 mg/kg to about 10 mg/kg. An indicated single dosage in the larger mammal, including, but not limited to, humans, is in the range from about 0.5 mg to about 1000 mg, conveniently administered in a single dose or in divided doses, including, but not limited to, up to four times a day or in extended release form. Suitable unit dosage forms for administration include from about 1 to about 500 mg active ingredient. In one embodiment, the unit dosage is about 1 mg, about 5 mg, about, 10 mg, about 20 mg, about 50 mg, about 100 mg, about 200 mg, about 250 mg, about 400 mg, or about 500 mg. In some embodiments, the an anti-programmed cell death protein 1 agent, an anti-programmed death ligand 1 agent, an anti-programmed death ligand 2 agent, a salt of any one thereof, or any combination thereof, may be 0.001% to 99% by weight of a pharmaceutical composition.

In one embodiment, a single dose of an immunotherapeutic agent is from about 0.1 mg/kg to about 10 mg/kg. In one embodiment, a single dose of blisibimod, belimumab, tabalumab, or a salt of any one thereof, is from about 0.1 mg/kg to about 10 mg/kg.

The foregoing ranges are merely suggestive, as the number of variables in regard to an individual treatment regime is large, and considerable excursions from these recommended values are not uncommon. Such dosages may be altered depending on a number of variables, not limited to the activity of the pharmaceutical composition used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. The data obtained from cell culture assays and animal studies can be used in formulating a range of dosage for use in human. The dosage may vary within this range depending upon the dosage form employed and the route of administration utilized.

The method used for cancer therapy with a pharmaceutical composition described herein may comprise one or more of a decreased single dose, decreased cumulative dose over a single therapeutic cycle, or decreased cumulative dose of the pharmaceutical composition over multiple therapeutic cycles compared with the amount required for cancer therapy.

The treatment regimen of the methods for cancer therapy comprises administering a pharmaceutical composition for a time sufficient and in an amount sufficient that kills tumor cells. In certain embodiments, the pharmaceutical composition is administered within a treatment cycle, which treatment cycle comprises a treatment course followed by a non-treatment interval. A treatment course of administration refers herein to a finite time frame over which one or more doses of the pharmaceutical composition on one or more days are administered. The finite time frame may be also called herein a treatment window.

Ingredients of a pharmaceutical composition described herein may be administered concurrently (e.g., simultaneously, essentially simultaneously or within the same treatment protocol) or sequentially. In some embodiments, an immunotherapeutic agent and a checkpoint inhibitor are administered concurrently. In some embodiments, an immunotherapeutic agent and a checkpoint inhibitor are administered sequentially. In some embodiments, an immunotherapeutic agent and a checkpoint inhibitor are administered within the same formulation. In some embodiments, an immunotherapeutic agent and a checkpoint inhibitor are administered in different formulations within a single treatment schedule, or the immunotherapeutic agent and a checkpoint inhibitor are administered on different treatment schedules.

In some embodiments, a composition of the current disclosure can be made by contacting combining, mixing, adding, or any combination thereof, an anti-PD-1, an anti-PD-L1, an anti-PD-L2, or a salt of any one thereof, or any combination thereof, and an immunotherapeutic agent or a salt thereof. In some embodiments, a kit of the current disclosure can be made by contacting combining, mixing, adding, or any combination thereof, an anti-PD-1, an anti-PD-L1, an anti-PD-L2, or a salt of any one thereof, or any combination thereof, and an immunotherapeutic agent or a salt thereof.

In a particular embodiment, a method of the current disclosure comprises administering the pharmaceutical composition in at least two treatment cycles. In a specific embodiment, the non-treatment interval may be at least about 2 weeks or between at least about 0.5-12 months, such as at least about one month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months (i.e., 1 year). In certain embodiments, the non-treatment interval is between 1-2 years or between 1-3 years, or longer. In certain embodiments, each treatment course is no longer than about 1 month, no longer than about 2 months, or no longer than about 3 months; or is no longer than 1, 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days.

In certain embodiments, the treatment window (i.e., treatment course) is only one day. In other certain embodiments, a single treatment course occurs over no longer than 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days. During such treatment windows, the pharmaceutical composition may be administered at least on two days (i.e., two days or more) with a variable number of days on which the agent is not administered between the at least two days of administration. Stated another way, within a treatment course when the pharmaceutical composition is administered on two or more days, the treatment course may have one or more intervals of one or more days when the pharmaceutical composition, is not administered. By way of non-limiting example, when the pharmaceutical composition is administered on 2 or more days during a treatment course not to exceed 21 days, the agent may be administered on any total number of days between from 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days. In certain embodiments, the pharmaceutical composition is administered to a subject during a treatment course of 3 days or more, and the agent may be administered every 2^{nd} day (i.e., every other day). In certain embodiments, when the pharmaceutical composition is administered to a subject for a treatment window of 4 days or more, the pharmaceutical composition may be administered every 3^{rd} day. In one embodiment, the pharmaceutical composition is administered on at least two days during a treatment course that is at least 2 days and no more than about 21 days (i.e., from about 2-21 days); at least 2 days and no longer than about 14 days (i.e., from about 2-14 days); at least 2 days and no longer than about 10 days (i.e., from about 2-10 days); or at least 2 days and no longer than about 9 days (i.e., from about 2-9 days); or at least 2 days and no longer than about 8 days (i.e., from about 2-8 days). In other specific embodiments, the pharmaceutical composition is administered on at least two days (i.e., 2 or more) during a treatment window is at least 2 days and no longer than about 7 days (i.e., from about 2-7 days); at least 2 days and no longer than about 6 days (i.e., from about 2-6 days) or at least 2 days and no more than about 5 days (i.e., from about 2-5 days) or at least 2 days and no longer than about 4 days (i.e., from about 2-4 days). In yet another embodiment, the treatment window is at least 2 days and no longer than 3 days (i.e., 2-3 days), or 2 days. In certain particular embodiments, the treatment course is no longer than 3 days. In other embodiments, the treatment course is no longer than 5 days. In still other specific embodiments, the treatment course is no longer than 7 days, 10 days, or 14 days or 21 days. In certain embodiments, the pharmaceutical composition is administered on at least two days (i.e., 2 or more days) during a treatment window that is at least 2 days and no longer than about 11 days (i.e., 2-11 days); or the pharmaceutical composition is administered on at least two days (i.e., 2 or more days) during a treatment window that is at least 2 days and no longer than about 12 days (i.e., 2-12 days); or the pharmaceutical composition is administered on at least two days (i.e., 2 or more days) during a treatment window that is at least 2 days and no more than about 13 days (i.e., 2-13 days); or the pharmaceutical composition is administered on at least two days (i.e., 2 or more days) during a treatment course that is at least 2 days and no more than about 15 days (i.e., 2-15 days); or the pharmaceutical composition is administered on at least two days (i.e., 2 or more days) during a treatment course that is at least 2 days and no longer than about 16 days, 17 days, 18 days, 19 days, or 20 days (i.e., 2-16, 2-17, 2-18, 2-19, 2-20 days, respectively). In other embodiments, the pharmaceutical composition may be administered on at least 3 days over a treatment course of at least 3 days and no longer than any number of days between 3 and 21 days; or is administered on at least 4 days over a treatment course of at least 4 days and no longer than any number of days between 4 and 21 days; or is administered on at least 5 days over a treatment course of at least 5 days and no longer than any number of days between 5 and 21 days; or is administered on at least 6 days over a treatment course of at least 6 days and no longer than any number of days between 6 and 21 days; or is administered at least 7 days over a treatment course of at least 7 days and no longer than any number of days between 7 and 21 days; or is administered at least 8 or 9 days over a treatment course of at least 8 or 9 days, respectively, and no longer than any number of days between 8 or 9 days, respectively, and 21 days; or is administered at least 10 days over a treatment course of at least 10 days and no longer than any number of days between 10 and 21 days; or is administered at least 14 days over a treatment course of at least 14 days and no longer than any number of days between 14 and 21 days; or is administered at least 11 or 12 days over a treatment course of at least 11 or 12 days, respectively, and no longer than any number of days between 11 or 12 days, respectively, and 21 days; or is administered at least 15 or 16 days over a treatment course of at least 15 or 16 days, respectively, and no longer than any number of days between 15 or 16 days, respectively, and 21 days. By way of additional example, when the treatment course is no longer than 14 days, a pharmaceutical composition may be administered on at least 2, 3, 4, 5, 6, 7, 8, 9, 10, 11, 12, 13, and 14 days over a treatment of window of at least 2, 3, 4, 5, 6, 7, 8, 9, 10, 11, 12, 13, and 14 days, respectively, and no longer than 14 days. When the treatment course is no longer than 10 days, a pharmaceutical composition may be administered on at least 2, 3, 4, 5, 6, 7, 8, 9, or 10 days over a treatment of window of at least 2, 3, 4, 5, 6, 7, 8, 9, or 10 days, respectively, and no longer than 10 days. Similarly, when the treatment course is no longer than 7 days, a pharmaceutical composition may be administered on at least 2, 3, 4, 5, 6, or 7 days over a treatment window of at least 2, 3, 4, 5, 6, or 7 days, respectively, and no longer than 7 days. In still another example, when the treatment course is no longer than 5 days, a pharmaceutical composition may be administered on at least 2, 3, 4, or 5 days over a treatment of window of at least 2, 3, 4, or 5 days, respectively, and no longer than 5 days.

With respect to a treatment course of three or more days, doses of the pharmaceutical composition may be administered for a lesser number of days than the total number of days within the particular treatment window. By way of non-limiting example, when a course of treatment has a treatment course of no more than 7, 10, 14, or 21 days, the number of days on which the pharmaceutical composition may be administered is any number of days between 2 days and 7, 10, 14, or 21 days, respectively, and at any interval appropriate for the particular disease being treated, the pharmaceutical composition being administered, the health status of the subject and other relevant factors, which are discussed in greater detail herein. A person may appreciate that when the pharmaceutical composition is administered on two or more days over a treatment window, the agent may be delivered on the minimum number days of the window, the maximum number of days of the window, or on any number of days between the minimum and the maximum.

In certain specific embodiments, a treatment course is one day or the treatment course is of a length not to exceed 2, 3, 4, 5, 6, 7, 8, 9, 10, 11, 12, 13, or 14 days, which are examples of a course wherein the pharmaceutical composition is administered on two or more days over a treatment course not to exceed (i.e., no longer than) 2, 3, 4, 5, 6, 7, 8, 9, 10, 11, 12, 13, or 14 days, respectively. In other certain embodiments, the treatment course is about 2 weeks (about 14 days or 0.5 months), about 3 weeks (about 21 days), about 4 weeks (about one month), about 5 weeks, about 6 weeks (about 1.5 months), about 2 months (or about 60 days), or about 3 months (or about 90 days). In a particular embodiment, a treatment course is a single daily dosing of the pharmaceutical composition. In other embodiments, with respect to any treatment course a daily dose of the pharmaceutical composition may be as a single administration or the dose may be divided into 2, 3, 4, or 5 separate administrations to provide the total daily dose of the agent.

As described herein, in certain specific embodiments, within a treatment window when the pharmaceutical composition is administered on two are more days, the treatment course may have one or more intervals of one or more days when the pharmaceutical composition, is not administered. Solely as a non-limiting example, when a treatment window is between two and seven days, a first dose may be administered on the first day of the treatment window and a second dose may be administered on the third day of the course, and a third dose may be administered on the seventh day of the treatment window. A person may appreciate that varying dosing schedules may be used during a particular treatment window. In other specific embodiments, the pharmaceutical composition is administered daily on each consecutive day for the duration of the treatment course. A daily dose may be administered as a single dose or the daily dose may be divided into 2, 3, or 4, or 5 separate administrations to provide the total daily dose of the pharmaceutical composition.

In certain embodiments, the treatment course comprises a length of time during which the pharmaceutical composition is administered daily. In one specific embodiment, the pharmaceutical composition is administered daily for 2 days. In another specific embodiment, the pharmaceutical composition is administered daily for 3 days. In yet another particular embodiment, the pharmaceutical composition is administered daily for 4 days. In one specific embodiment, the pharmaceutical composition is administered daily for 5 days. In yet another particular embodiment, the pharmaceutical composition is administered daily for 6 days. In another specific embodiment, the pharmaceutical composition is administered daily for 7 days. In yet another particular embodiment, the pharmaceutical composition is administered daily for 8 days. In still another specific embodiment, the pharmaceutical composition is administered daily for 9 days. In yet another particular embodiment, the pharmaceutical composition is administered daily for 10 days. In yet another particular embodiment, the pharmaceutical composition is administered daily for 11 days. In yet another particular embodiment, the pharmaceutical composition is administered daily for 12 days. In yet another particular embodiment, the pharmaceutical composition is administered daily for 13 days. In yet another particular embodiment, the pharmaceutical composition is administered daily for 14 days. The treatment window (i.e., course) for each of the above examples is no longer than 2, 3, 4, 5, 6, 7, 8, 9, 10, 11, 12, 13, 14 days, respectively.

In other specific embodiments, the pharmaceutical composition is administered every 2^{nd} day (i.e., every other day) for 3, 4, 5, 6, 7, 8, 9, 10, 11, 12, 13, or 14 days. In still other specific embodiments, the pharmaceutical composition is administered every 3^{nd} day (i.e., one day receiving the agent followed by two days without receiving the agent) for 4, 5, 6, 7, 8, 9, 10, 11, 12, 13, or 14 days. In still other specific embodiments, the pharmaceutical composition may be administered on every 2^{nd}-3^{rd} day during a treatment window of 3, 4, 5, 6, 7, 8, 9, 10, 11, 12, 13, or 14 days. In yet other embodiments, the pharmaceutical composition may be administered every 4^{th} day during a treatment course of 5, 6, 7, 8, 9, 10, 11, 12, 13, or 14 days; or every 5^{th} day during a treatment course of 6, 7, 8, 9, 10, 11, 12, 13, or 14 days. A person may appreciate the minimum numbers of days in a treatment window when the pharmaceutical composition is administered every 6^{th}, 7^{th}, etc. day over a treatment window of a finite number of days as described herein.

In certain particular embodiments, a pharmaceutical composition may be administered daily for a longer duration than 14 days and may be administered at least 15, 16, 17, 18, 19, 20, or at least 21 days. In other specific embodiments, the pharmaceutical composition may be administered daily on each of the 15, 16, 17, 18, 19, 20, or 21 days. In another specific embodiment, the pharmaceutical composition may be administered every second day during a treatment window of 15, 16, 17, 18, 19, 20, or 21 days. In another specific embodiment, the pharmaceutical composition may be administered every third day during a treatment window of 15, 16, 17, 18, 19, 20, or 21 days. In still other specific embodiments, the pharmaceutical composition may be administered on every 2^{nd}-3^{rd} day during a treatment window of 15, 16, 17, 18, 19, 20, or 21 days. In yet other embodiments, the pharmaceutical composition may be administered every 4^{th} day during a treatment course of 15, 16, 17, 18, 19, 20, or 21 days; or every 5^{th} day during a treatment course of 15, 16, 17, 18, 19, 20, or 21 days. A person can readily appreciate the minimum numbers of days in a treatment window when the pharmaceutical composition is administered every 6^{th}, 7^{th}, etc. day over a treatment window of a finite number of days as described herein.

In another certain particular embodiment, a pharmaceutical composition may be administered daily for a longer duration than 14 days and may be administered at least 15, 16, 17, 18, 19, 20, or at least 21 days. In other specific embodiments, the pharmaceutical composition may be administered daily on each of the 15, 16, 17, 18, 19, 20, or 21 days. In another specific embodiment, the pharmaceutical composition may be administered every second day during a treatment window of 15, 16, 17, 18, 19, 20, or 21 days. In another specific embodiment, the pharmaceutical composition may be administered every third day during a treatment window of 15, 16, 17, 18, 19, 20, or 21 days. In still other specific embodiments, the pharmaceutical composition may be administered on every 2^{nd}-3^{rd} day during a treatment window of 15, 16, 17, 18, 19, 20, or 21 days. In yet other embodiments, the pharmaceutical composition may be administered every 4^{th} day during a treatment course of 15, 16, 17, 18, 19, 20, or 21 days; or every 5^{th} day during a treatment course of 15, 16, 17, 18, 19, 20, or 21 days. A person can readily appreciate the minimum numbers of days in a treatment window when the pharmaceutical composition is administered every 6^{th}, 7^{th}, etc. day over a treatment window of a finite number of days as described herein.

In another certain particular embodiment, a pharmaceutical composition may be administered in a treatment course daily for a longer duration than 14 days or 21 days and may be administered in a treatment course of about one month, about two months, or about three months. In other specific embodiments, the pharmaceutical composition may be administered daily on each of a one month, two month, or three month treatment course. In another specific embodiment, the pharmaceutical composition may be administered every second day during a treatment course of about one month, about two months, or about three months. In another specific embodiment, the pharmaceutical composition may be administered every third day during a treatment course of about one month, about two months, or about three months. In still other specific embodiments, the pharmaceutical composition may be administered on every 2^{nd}-3^{rd} day during a treatment course of about one month, about two months, or about three months. In yet other embodiments, the pharmaceutical composition may be administered every 4^{th} day during a treatment course of about one month, about two months, or about three months; or every 5^{th} day during a treatment course of about one month, about two months, or about three months s. A person can readily appreciate the minimum numbers of days in a treatment course when the pharmaceutical composition is administered every 6^{th}, 7^{th}, etc. day over a treatment window of a finite number of days as described herein.

By way of non-limiting example, a longer treatment window with a decreased dose per day may be a treatment option for a subject. In other particular embodiments and by way of example, the stage or severity of the cancer or tumor may indicate that a longer term course may provide clinical benefit. In certain embodiments, the pharmaceutical composition is administered daily, or optionally, every other day (every 2^{nd} day) or every 3^{rd} day, or greater interval (i.e., every 4^{th} day, 5^{th} day, 6^{th} day) during a treatment course of about 1-2 weeks (e.g., about 5-14 days), about 1-3 weeks (e.g., about 5-21 days), about 1-4 weeks (e.g., about 5-28 days, about 5-36 days, or about 5-42 days, 7-14 days, 7-21 days, 7-28 days, 7-36 days, or 7-42 days; or 9-14 days, 9-21 days, 9-28 days, 9-36 days, or 9-42 days. In other certain embodiments, the treatment course is between about 1-3 months. In a specific embodiment, the pharmaceutical composition is administered daily for at least five days, and in another particular embodiment, the pharmaceutical composition is administered daily for 5-14 days. In other particular embodiments, the pharmaceutical composition is administered for at least seven days, for example, for 7-14, 7-21, 7-28 days, 7-36 days, or 7-42 days. In other particular embodiments, the pharmaceutical composition is administered for at least nine days, for example, for 9-14 days, 9-21 days, 9-28 days, 9-36 days, or 9-42 days.

Even though as discussed herein and above, a treatment course comprising administering a pharmaceutical composition provides clinical benefit, in other certain embodiments, a treatment course is repeated with a time interval between each treatment course when the pharmaceutical composition is not administered (i.e., non-treatment interval, off-drug treatment). A treatment cycle as described herein and in the art comprises a treatment course followed by a non-treatment interval. A treatment cycle may be repeated as often as needed. For example, a treatment cycle may be repeated at least once, at least twice, at least three times, at least four times, at least five times, or more often as needed. In certain specific embodiments, a treatment cycle is repeated once (i.e., administration of the pharmaceutical composition comprises 2 treatment cycles). In other certain embodiments, the treatment cycle is repeated twice or repeated 3 or more times. Accordingly, in certain embodiments, one, two, three, four, five, six, seven, eight, nine, ten, or more treatment cycles of treatment with a pharmaceutical composition are performed. In particular embodiments, a treatment course or a treatment cycle may be repeated, such as when the cancer or tumor recurs, or when symptoms or sequelae of the disease or disorder that were significantly diminished by one treatment course as described above have increased or are detectable, or when the symptoms or sequelae of the disease or disorder are exacerbated, a treatment course may be repeated. In other embodiments when the pharmaceutical composition is administered to a subject to prevent (i.e., reduce likelihood of occurrence or development) or to delay onset, progression, or severity of the cancer or tumor, a subject may receive the pharmaceutical composition over two or more treatment cycles. Accordingly, in certain embodiments, one cycle of treatment is followed by a subsequent cycle of treatment. Each treatment course of a treatment cycle or each treatment course of two or more treatment cycles are typically the same in duration and dosing of the pharmaceutical composition. In other embodiments, the duration and dosing of the pharmaceutical composition during each treatment course of a treatment cycle may be adjusted as determined by a person skilled in the medical art depending, for example, on the particular disease or disorder being treated, the pharmaceutical composition being administered, the health status of the subject and other relevant factors, which are discussed in greater detail herein. Accordingly, a treatment course of a second or any subsequent treatment cycle may be shortened or lengthened as deemed medically necessary or prudent. In other words, as would be appreciated by a person, each treatment course of two or more treatment cycles are independent and the same or different; and each non-treatment interval of each treatment cycle is independent and the same or different.

As described herein, each course of treatment in a treatment cycle is separated by a time interval of days, weeks, or months without treatment with a pharmaceutical composition (i.e., non-treatment time interval or off-drug interval; called non-treatment interval herein). The non-treatment interval (such as days, weeks, months) between one treatment course and a subsequent treatment course is typically greater than the longest time interval (i.e., number of days) between any two days of administration in the treatment course. By way of example, if a treatment course is no longer than 14 days and the agent is administered every other day during this treatment course, the non-treatment interval between two treatment courses is greater than 2 days, such as 3, 4, 5, 6, 7, 8, 9, 10, 11, 12, 13, or 14 days or about 3 weeks, about 4 weeks, about 6 weeks, or about 2 months or longer as described herein. In particular embodiments, the non-treatment interval between two treatment courses is about 5 days, about 1 week, about 2 weeks, about 3 weeks, about 1 month, about 6 weeks, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In certain specific embodiments, the non-treatment interval is about 2 years or about 3 years. In certain specific embodiments, the non-treatment time interval is at least about 14 days, at least about 21 days, at least about 1 month, at least about 2 months, at least about 3 months, at least about 4 months, at least about 5 months, at least about 6 months, or at least about 1 year. In certain embodiments, a course of treatment (whether daily, every other day, every 3^{rd} day, or other interval between administrations within the treatment course as described above (e.g., 1-14 days, 2-14 days, 2-21 days, or 1-21 days)) is administered about every 14 days (i.e., about every 2 weeks) (i.e., 14 days without pharmaceutical composition treatment), about every 21 days (i.e., about every 3 weeks), about every 28 days (i.e., about every 4 weeks), about every one month, about every 36 days, about every 42 days, about every 54 days, about every 60 days, or about every month (about every 30 days), about every two months (about every 60 days), about every quarter (about every 90 days), or about semi-annually (about every 180 days). In other certain embodiments, a course of treatments (e.g., by way of non-limiting example, administration on at least one day or on at least two days during a course for about 2-21 days, about 2-14, days, about 5-14 days, about 7-14 days, about 9-14 days, about 5-21 days, about 7-21 days, about 9-21 days) is administered every 28 days, every 36 days, every 42 days, every 54 days, every 60 days, or every month (about every 30 days), every two months (about every 60 days), every quarter (about every 90 days), or semi-annually (about every 180 days), or about every year (about 12 months). In other embodiments, a course of treatment (such as by way of non-limiting examples, e.g., for about 5-28 days, about 7-28 days, or about 9-28 days whether daily, every other day, every 3^{rd} day, or other interval between administrations within the treatment course) is administered every 36 days, 42 days, 54 days, 60 days, or every month (about every 30 days), every two months (about every 60 days), every quarter (about every 90 days), or semi-annually (about every 180 days). In other particular embodiments, a course of treatment (e.g., for about 5-36 days, 7-36 days, or 9-36 days whether daily, every other day, every 3^{rd} day, or other interval between administrations within the treatment course) is administered every 42 days, 54 days, 60 days, or every month (about every 30 days), every two months (about every 60 days), every quarter (about every 90 days), or semi-annually (about every 180 days), or about every year (about 12 months).

In a particular embodiment, the treatment course is one day and the non-treatment interval is at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In other certain embodiments, the treatment course is at least two days or is at least 3 days and no longer than 10 days, and the non-treatment interval is at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In still another embodiment, the treatment course is at least three days and no longer than 10 days, no longer than 14 days, or no longer than 21 days, and the non-treatment interval is at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In still another embodiment, a treatment course (e.g., for about 5-42, 7-42, or 9-42 days whether daily, every other day, every 3^{rd} day, or other interval between administrations within the treatment course) is administered every 42 days, 60 days, or every month (about every 30 days), every two months (about every 60 days), every quarter (about every 90 days), or semi-annually (about every 180 days), or about every year (about 12 months). In a particular embodiment, the pharmaceutical composition is administered daily for 5-14 days every 14 days (about every 2 weeks), or every 21-42 days. In another particular embodiment, the pharmaceutical composition is administered daily for 5-14 days quarterly. In another particular embodiment, the pharmaceutical composition is administered daily for 7-14 days every 21-42 days. In another particular embodiment, the pharmaceutical composition is administered daily for 7-14 days quarterly. In still other particular embodiments, the pharmaceutical composition is administered daily for 9-14 days every 21-42 days or every 9-14 days quarterly. In still other embodiments, the non-treatment interval may vary between treatment courses. By way of non-limiting example, the non-treatment interval may be 14 days after the first course of treatment and may be 21 days or longer after the second, third, or fourth (or more) course of treatment. In other particular embodiments, the pharmaceutical composition is administered to the subject in need thereof once every 0.5-12 months. In other certain embodiments, the pharmaceutical composition is administered to the subject in need once every 4-12 months.

In certain embodiments, a pharmaceutical composition is administered to a subject to reduce the likelihood or the risk that the subject will develop a cancer. In certain embodiments, the pharmaceutical composition is administered for one or more days (e.g., any number of consecutives days between and including 2-3, -4, -5, -6, -7, -8, -9, -10, -11, -12, -13, -14, -15, -16, -17, -18, -19, -20, and 2-21 days) every 3, 4, 5, 6, 7, 8, 9, 10, 11, or 12 months. In a particular embodiment, the pharmaceutical composition is administered for one or more days (e.g., any number of consecutives days between and including 1-9 days) every 5 or 6 months.

The total daily dose of a pharmaceutical composition may be delivered as a single dose or as multiple doses on each day of administration. In other certain particular embodiments, when multiple cycles of the pharmaceutical composition are administered, the dose of a pharmaceutical composition administered on a single day may be less than the daily dose administered if only a single treatment course is intended to be administered.

### Combination Therapies

A composition comprising a therapeutically effective amount of an anti-programmed cell death protein 1 agent and an immunotherapeutic agent, may also be used in combination with other therapeutic agents that are selected for their therapeutic value for the condition to be treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, may be within the knowledge of the clinician. The initial administration can be made according to established protocols recognized in the field, and then, based upon the observed effects, the dosage, modes of administration and times of administration can be modified by the clinician.

In certain instances, it may be appropriate to administer at least one pharmaceutical composition described herein in combination with another therapeutic agent. Or, by way of example only, the therapeutic effectiveness of one of the pharmaceutical compositions described herein may be enhanced by administration of an adjuvant, i.e., by itself the adjuvant may have minimal therapeutic benefit, but in combination with another therapeutic agent, the overall therapeutic benefit to the subject is enhanced. Or, by way of example only, the benefit experienced by a subject may be increased by administering one of the pharmaceutical compositions described herein with another therapeutic agent, which also includes a therapeutic regimen that also has therapeutic benefit. In any case, regardless of the disease, disorder or condition being treated, the overall benefit experienced by the subject may simply be additive of the two agents or the subject may experience a synergistic benefit.

The particular choice of pharmaceutical compositions used will depend upon the diagnosis of the attending physicians and their judgment of the condition of the subject and the appropriate treatment protocol. The pharmaceutical compositions may be administered concurrently (e.g., simultaneously, essentially simultaneously or within the same treatment protocol) or sequentially, depending upon the nature of the disease, disorder, or condition, the condition of the subject, and the actual choice of pharmaceutical compositions used. The determination of the order of administration, and the number of repetitions of administration of each therapeutic agent during a treatment protocol is well within the knowledge of the physician after evaluation of the disease being treated and the condition of the subject.

Therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the subject.

For combination therapies described herein, dosages of the co-administered pharmaceutical compositions will vary depending on the type of co-drug employed, on the specific drug employed, on the disease or condition being treated and so forth. In addition, when co-administered with one or more biologically active agents, the pharmaceutical composition provided herein may be administered either simultaneously with the pharmaceutical composition, or sequentially. If administered sequentially, the attending physician will decide on the appropriate sequence of administering the pharmaceutical composition in combination with a biologically active agent(s).

In one embodiment, the composition comprising a therapeutic therapeutically effective amount of an anti-programmed cell death protein 1 agent and an immunotherapeutic agent may be administered in any order or even simultaneously. In one embodiment, the composition comprising a therapeutic therapeutically effective amount of an anti-programmed cell death protein 1 agent and an immunotherapeutic agent may be administered in any order or even simultaneously, wherein the immunotherapeutic agent is tabalumab or a fragment or a salt thereof. If simultaneously, the multiple therapeutic agents may be provided in a single, unified form, or in multiple forms, by way of example only, either as a single pill or as two separate pills. One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; the use of multiple therapeutic combinations are also envisioned.

It is understood that the dosage regimen to treat, prevent, or ameliorate the condition(s) for which relief is sought, can be modified in accordance with a variety of factors. These factors include the disorder or condition from which the subject suffers, as well as the age, weight, sex, diet, and medical condition of the subject. Thus, the dosage regimen actually employed can vary widely and therefore can deviate from the dosage regimens set forth herein.

The pharmaceutical compositions which make up the combination therapy disclosed herein may be a combined dosage form or in separate dosage forms intended for substantially simultaneous administration. The pharmaceutical compositions that make up the combination therapy may also be administered sequentially, with either pharmaceutical composition being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical composition, such as potency, solubility, bioavailability, plasma half-life and kinetic profile of the pharmaceutical composition. Circadian variation of the target molecule concentration may also determine the optimal dose interval.

In addition, the pharmaceutical compositions described herein also may be used in combination with procedures that may provide additional or synergistic benefit to the subject. By way of example only, subjects are expected to find therapeutic and/or prophylactic benefit in the methods described herein, wherein pharmaceutical composition of a pharmaceutical composition disclosed herein and/or combinations with other therapeutics are combined with genetic testing to determine whether that individual is a carrier of a mutant gene that may be known to be correlated with certain diseases or conditions.

The pharmaceutical compositions described herein and combination therapies can be administered before, during or after the occurrence of a disease or condition, and the timing of administering the composition containing a pharmaceutical composition can vary. Thus, for example, the pharmaceutical composition can be used as a prophylactic and can be administered continuously to subjects with a propensity to develop conditions or diseases in order to prevent the occurrence of the disease or condition. The pharmaceutical compositions and compositions can be administered to a subject during or as soon as possible after the onset of the symptoms. The administration of the pharmaceutical compositions can be initiated within the first 48 hours of the onset of the symptoms, such as within the first 48 hours of the onset of the symptoms, such as within the first 6 hours of the onset of the symptoms, such as within 3 hours of the onset of the symptoms. The initial administration can be via any route practical, such as, for example, an intravenous injection, a bolus injection, infusion over about 5 minutes to about 5 hours, a pill, a capsule, transdermal patch, buccal delivery, and the like, or combination thereof. A pharmaceutical composition may be administered as soon as is practicable after the onset of a disease or condition is detected or suspected, and for a length of time necessary for the treatment of the disease, such as, for example, from 1 day to about 3 months. The length of treatment can vary for each subject, and the length can be determined using the known criteria. For example, the pharmaceutical composition or a formulation containing the pharmaceutical composition can be administered for at least 2 weeks, such as about 1 month to about 5 years.

A pharmaceutical composition described herein can reduce the likelihood of a cancer in a subject in need thereof. The pharmaceutical composition described herein can be administered one or more days within a window of treatment. In some embodiments, the treatment window is 1, 2, 3, 4, 5, 6, 7, 8, 9, 10, 11, 12, 13, 14, 15, 16, 17, 18, 19, 20, or 21 days. In some embodiments, a pharmaceutical composition described herein is administered on two or more days within a treatment window of no longer than 7 days or 14 days; on 3 or more days within a treatment window of no longer than 7 days or 14 days; on 4 or more days within a treatment window of no longer than 7 days or 14 days; on 5 or more days within a treatment window of no longer than 7 days or 14 days; or on 6, 7, 8, 9, 10, 11, 12, 13, or 14 days within treatment window of no longer than 7 days or 14 days.

Chemotherapy and radiotherapy treatment regimens can comprise a finite number of cycles of on-drug therapy followed by off-drug therapy, or comprise a finite timeframe in which the chemotherapy or radiotherapy is administered. The protocols can be determined by clinical trials, drug labels, and clinical staff in conjunction with the subject to be treated. The number of cycles of a chemotherapy or radiotherapy or the total length of time of a chemotherapy or radiotherapy regimen can vary depending on the subject's response to the cancer therapy. A pharmaceutical composition described herein can be administered after the treatment regimen of chemotherapy or radiotherapy has been completed.

### Indications

In some embodiments, a method of the current disclosure is used to treat a cancer or a tumor. In some embodiments, the cancer or tumor is malignant. In some embodiments, the cancer or tumor is present in an organ. In some embodiments, the cancer or tumor is present in the head or neck region, the abdominal region, an upper limb, a lower limb, the skin, blood, the digestive tract, a germ cell, or the nervous system of a subject. In some embodiments, the cancer or tumor is present in at least one of: the blood, the lymph, or the cerebral spinal fluid.

In some embodiments, the tumor is a solid tumor. In some embodiments, the tumor is a liquid tumor. Cancers that are liquid tumors can be those that occur, for example, in blood, bone marrow, and lymph nodes, and can include, for example, leukemia, myeloid leukemia, lymphocytic leukemia, lymphoma, Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CML), and hairy cell leukemia. Cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In some embodiments, the condition treated by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

In some embodiments, a method of the current disclosure may be used to treat cancer, wherein the cancer is prostate cancer, testicular cancer, breast cancer, brain cancer, pancreatic cancer, colon cancer, thyroid cancer, stomach cancer, lung cancer, melanoma, multiple myeloma, Hodgkin's lymphoma, or ovarian cancer.

The methods described herein can also be used for inhibiting progression of metastatic cancer tumors. Non-limiting examples of cancers include adrenocortical carcinoma, childhood adrenocortical carcinoma, aids-related cancers, anal cancer, appendix cancer, basal cell carcinoma, childhood basal cell carcinoma, bladder cancer, childhood bladder cancer, bone cancer, brain tumor, childhood astrocytomas, childhood brain stem glioma, childhood central nervous system atypical teratoid/rhabdoid tumor, childhood central nervous system embryonal tumors, childhood central nervous system germ cell tumors, childhood craniopharyngioma brain tumor, childhood ependymoma brain tumor, breast cancer, childhood bronchial tumors, carcinoid tumor, childhood carcinoid tumor, gastrointestinal carcinoid tumor, carcinoma of unknown primary, childhood carcinoma of unknown primary, childhood cardiac tumors, cervical cancer, childhood cervical cancer, childhood chordoma, chronic myeloproliferative disorders, colon cancer, colorectal cancer, childhood colorectal cancer, extrahepatic bile duct cancer, ductal carcinoma in situ (DCIS), endometrial cancer, esophageal cancer, childhood esophageal cancer, childhood esthesioneuroblastoma, eye cancer, malignant fibrous histiocytoma of bone, gallbladder cancer, gastric (stomach) cancer, childhood gastric cancer, gastrointestinal stromal tumors (GIST), childhood gastrointestinal stromal tumors (GIST), childhood extracranial germ cell tumor, extragonadal germ cell tumor, gestational trophoblastic tumor, glioma, head and neck cancer, childhood head and neck cancer, hepatocellular cancer, hypopharyngeal cancer, kidney cancer, renal cell kidney cancer, Wilms tumor, childhood kidney tumors, Langerhans cell histiocytosis, laryngeal cancer, childhood laryngeal cancer, leukemia, acute lymphoblastic leukemia (ALL), acute myeloid leukemia (AML), chronic lymphocytic leukemia (CLL), chronic myelogenous leukemia (cml), hairy cell leukemia, lip cancer, liver cancer (primary), childhood liver cancer (primary), lobular carcinoma in situ (LCIS), lung cancer, non-small cell lung cancer, small cell lung cancer, lymphoma, aids-related lymphoma, burkitt lymphoma, cutaneous t-cell lymphoma, Hodgkin lymphoma, non-Hodgkin lymphoma, primary central nervous system lymphoma (CNS), melanoma, childhood melanoma, intraocular melanoma. Merkel cell carcinoma, malignant mesothelioma, childhood malignant mesothelioma, metastatic squamous neck cancer with occult primary, midline tract carcinoma involving NUT gene, mouth cancer, childhood multiple endocrine neoplasia syndromes, mycosis fungoides, myelodysplastic syndromes, myelodysplastic neoplasms, myeloproliferative neoplasms, multiple myeloma, nasal cavity cancer, nasopharyngeal cancer, childhood nasopharyngeal cancer, neuroblastoma, oral cancer, childhood oral cancer, oropharyngeal cancer, ovarian cancer, childhood ovarian cancer, epithelial ovarian cancer, low malignant potential tumor ovarian cancer, pancreatic cancer, childhood pancreatic cancer, pancreatic neuroendocrine tumors (islet cell tumors), childhood papillomatosis, paraganglioma, paranasal sinus cancer, parathyroid cancer, penile cancer, pharyngeal cancer, pheochromocytoma, pituitary tumor, plasma cell neoplasm, childhood pleumpulmonary blastoma, prostate cancer, rectal cancer, renal pelvis transitional cell cancer, retinoblastoma, salivary gland cancer, childhood salivary gland cancer, Ewing sarcoma family of tumors, Kaposi Sarcoma, osteosarcoma, rhabdomyosarcoma, childhood rhabdomyosarcoma, soft tissue sarcoma, uterine sarcoma, Sezary syndrome, childhood skin cancer, nonmelanoma skin cancer, small intestine cancer, squamous cell carcinoma, childhood squamous cell carcinoma, testicular cancer, childhood testicular cancer, throat cancer, thymoma and thymic carcinoma, childhood thymoma and thymic carcinoma, thyroid cancer, childhood thyroid cancer, ureter transitional cell cancer, urethral cancer, endometrial uterine cancer, vaginal cancer, vulvar cancer, and Waldenström macroglobulinemia.

In some embodiments, the cancer or tumor is squamous non-small cell lung cancer, adeno non-small cell lung cancer, colorectal cancer, head and neck squamous cell carcinoma, breast cancer, or melanoma.

In some embodiments, the cancer or tumor contacts a blood vessel. In some embodiments, the cancer or the tumor is in the interior of a blood vessel.

In some embodiments, at least one of the anti-programmed cell death protein 1 agent, the anti-programmed death ligand 1 agent, the anti-programmed death ligand 2 agent, or a salt of any one thereof, or any combination thereof; and the immunotherapeutic agent or salt thereof is isolated and purified.

In some embodiments, the administration is oral. In some embodiments, the administration is topical, intravenous, intramuscular, or spinal. In some embodiments, the administration is administered directly to the cancer or the tumor. In some embodiments, the administration is an administration at a location different than the cancer or the tumor. In some embodiments, the subject has been previously diagnosed with the cancer or the tumor. In some embodiments, the cancer or the tumor is a solid tumor or a solid cancer. In some embodiments, the cancer or the tumor is a liquid cancer or a liquid tumor. In some embodiments, the cancer or the tumor is malignant. In some embodiments, the cancer or the tumor is present in an organ. In some embodiments, the tumor or the cancer is present in at least one of: blood, lymph, cerebral spinal fluid. In some embodiments, the cancer or the tumor is located in a head or neck region, abdominal region, a upper limb, a lower limb, skin, blood, digestive tract, germ cell, or nervous system.

### Cancer Interactomes

In some embodiments, a method described herein may be used to define a cancer interactome in a tumor microencironment. A method may be used to correlate interaction partners of cells within a tumor.

To define a cancer interactome, a cell or a plurality of cells may be imaged. The cell or plurality of cells may be stained prior to imaging. An image may comprise at least 1, 2, 3, 4, 5, 6, 7, 8, 9, 10, 15, 20, 30, 40, 50, 60, 70, 80, 90, or 100 cells. In some embodiments, an image may comprise 1 to 10 cells, or 2-9 cells, or 3-8 cells. In some embodiments, an image may comprise 6 cells.

A cell image may comprise of a number of pixels. The number of pixels in a cell image may be at least about 100, 1,000, 10,000, 100,000, 1,000,000, 1,500.000, or 2,000,000 pixels. The number of pixels in a cell image may be at least about 1,500,000, pixels.

A matrix may be generated based on the stained plurality of cells. The matrix may be used to define or represent the boundaries of the cells. In some embodiments, an area that is a cell boundary may be defined using the number 1 in a matrix, and an area that is not a cell boundary may be defined using the number 0. Thus, a matrix may represent cellular segmentation.

A matrix may be generated to represent an intensity value of cells in a tumor microenvironment. The matrix may be determined from a fluorescence readout. The matrix may be generated by a human or by a computer or by a software program. The intensity matrix may be predetermined, or may be obtained from a reference sample. Mixed or unmixed images can be used as inputs that can be processed and formatted into unmixed matrixes.

The intensity of each cell may be calculated for total readout intensity. A reference intensity may exist, wherein the reference intensity may be based on a prior sample that has been designated as a standard. The intensity of a cell may be compared to a reference intensity. After comparison of the intensity of a cell to a reference intensity, the cell may be labeled as having a positive intensity. After comparison of the intensity of a cell to a reference intensity, the cell may be labeled as having a negative intensity.

A pixel adjacency graph may be generated. Pixel adjacency may be defined as any two pixels that are within a distance of square-root(2) pixels. Pixels that are directly adjacent or diagonally adjacent may be considered as having adjacency.

A cell adjacency graph may be generated. Cellular adjacency may be defined as any two cells that are within a distance of square-root(2) cells. Cells that are directly adjacent or diagonally adjacent may be considered as having adjacency.

An adjacency heatmap may be generated from a cell adjacency graph. The cell adjacency heatmap generated from a tumor sample of a subject may then be compared to a reference heatmap.

A reference heatmap may be generated prior to diagnosing a subject. Reference heatmaps may be generated and used as references to predict subject treatment outcome. Reference heatmaps may be used as references to diagnose subjects in need of treatment.

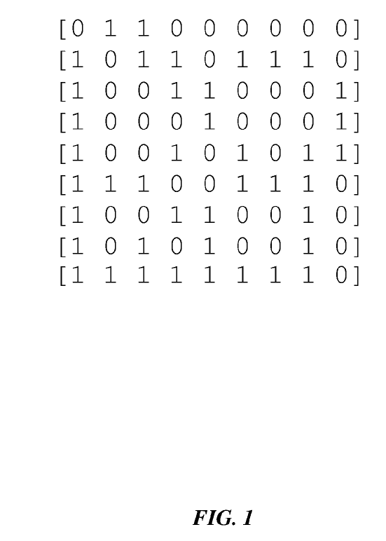

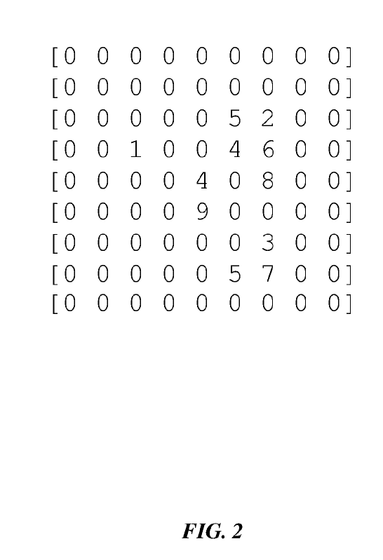

Two inputs may be used to define an interactome. The first is a matrix representing the cellular image where boundaries between cells are defined. FIG. 1 provides a representative example matrix that defines cellular boundaries. The matrix represents a 9×9 image which was determined to be comprised of six cells. FIG. 2 provides a 9×9 representative example which corresponds with FIG. 1 and is an example of an intensity matrix, wherein the relative intensity of each marker of interest per pixel is defined.

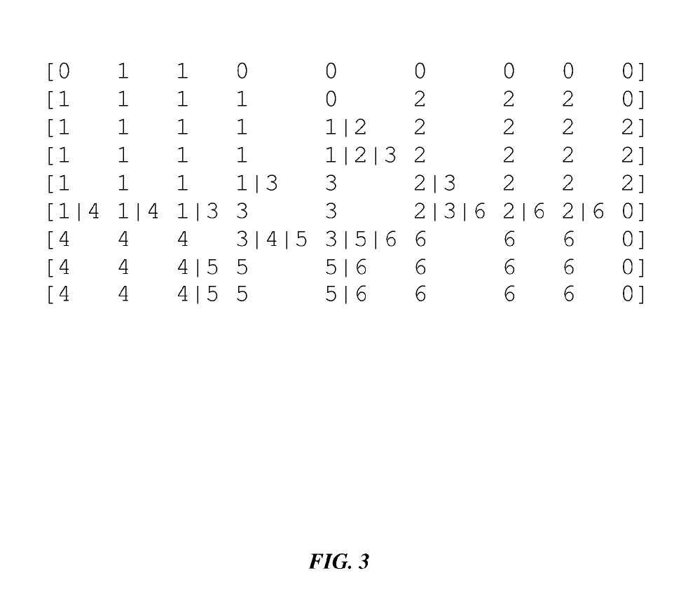

Based on the boundaries defined in the cellular boundary input, each pixel is labeled based on the cellular boundary with which it is contained. Boundary pixels are labeled based on all cells sharing that boundary pixel. An example of pixel labeling is shown in FIG. 3. The matrix represents cell labeling from the boundary information from FIG. 1, wherein the symbol “I” indicates pixels that are boundary pixels and labeled by the cells that are part of that boundary.

### Computer Control Systems

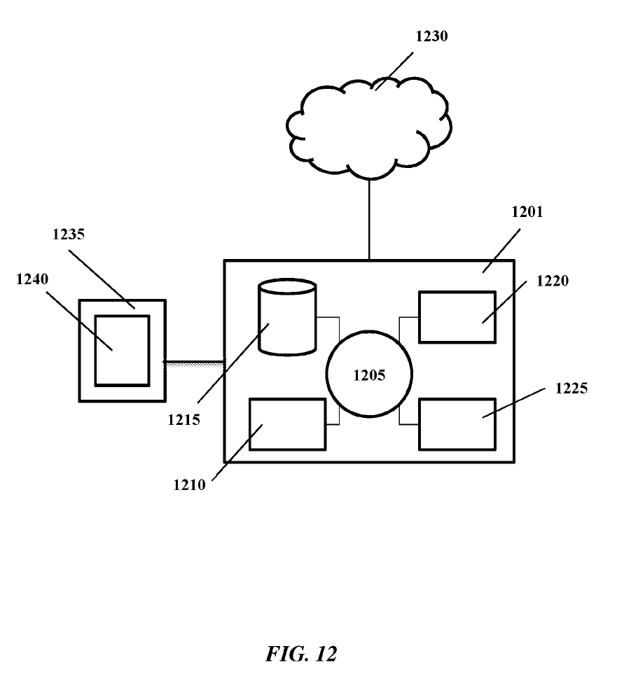

The present disclosure provides computer control systems that are programmed to implement methods of the disclosure. FIG. 12 shows a computer control system 1201. The computer control system 1201 can be implemented on an electronic device of a user or a computer system that is remotely located with respect to the electronic device. The electronic device can be a mobile electronic device.

The computer system 1201 includes a central processing unit (CPU, also “processor” and “computer processor” herein) 1205, which can be a single core or multi core processor, or a plurality of processors for parallel processing. The computer control system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer control system 1201 can be operatively coupled to a computer network (“network”) 1230 with the aid of the communication interface 1220. The network 1230 can be the Internet, an internet and/or extranet, or an intranet and/or extranet that is in communication with the Internet. The network 1230 in some cases is a telecommunication and/or data network. The network 1230 can include one or more computer servers, which can enable distributed computing, such as cloud computing. The network 1230, in some cases with the aid of the computer system 1201, can implement a peer-to-peer network, which may enable devices coupled to the computer system 1201 to behave as a client or a server.

The CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1201 can be included in the circuit. In some cases, the circuit is an application specific integrated circuit (ASIC).

The storage unit 1215 can store files, such as drivers, libraries and saved programs. The storage unit 1215 can store user data. e.g., user preferences and user programs. The computer system 1201 in some cases can include one or more additional data storage units that are external to the computer system 1201, such as located on a remote server that is in communication with the computer system 1201 through an intranet or the Internet.

The computer system 1201 can communicate with one or more remote computer systems through the network 1230. For instance, the computer system 1201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1201 via the network 1230.

Methods as described herein can be implemented by way of machine (e.g., computer processor) executable code stored on an electronic storage location of the computer system 1201, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 can include or be in communication with an electronic display 1235 that comprises a user interface (UI) 1240.

Methods and systems of the present disclosure can be implemented by way of one or more algorithms. An algorithm can be implemented by way of software upon execution by the central processing unit 1205.

### EXAMPLES

### Example 1: Staining a FFPE Tissue Section

A procedure is used to stain a FFPE tissue section mounted on a slide for multiple antibody/TSA-fluorophore conjugates. The immunohistochemistry staining is used with up to 6 different antibodies for multiple antigen detection on a tissue section on a single slide. This procedure allows quantitative analysis of antigens to identify biomarkers within a sample.

“IHC” refers to immunohistochemistry. “FFPE” refers to formalin fixed paraffin embedded. “TBS” refers to tris buffered saline. “TBS-T” refers to tris buffered saline with tween 20. “TSA” refers to tyramide signal amplification. “DAPI” refers to 4′, 6 diamidino-2-phenylindole, dihydrochloride.

A concentrated Hot Rinse at a ratio of 1:25 is formed (1 ml Hot Rinse to 24 ml of Deionized Water). A staining jar or metal slide canister is filled with 50-200 mL (enough volume to cover slides depending on size of staining jar) of 1× Hot Rinse. A second container is filled with 50-200 mL (enough volume to cover slides depending on size of staining jar) of 1× Borg Decloaker RTU. The decloaking Chamber is filled with 500 mL Deionized Water. The Borg Decloaker and Hot Rinse staining jars are placed into the Decloaking Chamber. The slides are placed into a slide rack, then the slide rack is placed into a staining jar containing Borg Decloaker. The decloaking chamber is heated at 110° C. for 15 minutes. The slide are transferred to the slide container with 1× Hot Rinse and dunked vigorously for a minimum of 20 times. A rinse is preformed by gradually adding 500 mL of deionized water to the solution. One fourth of the hot rinse is poured out, and deionized water is added again. This is repeated until all of the deionized water is used.

The primary antibody is prepared prior to use for fluorescent IHC staining.

The slides are transferred to 1×TBS. 2.5 ml of 30% hydrogen peroxide in 247.5 ml of deionized water is added. The slides are transferred to 0.3% hydrogen peroxide and incubated for 10 minutes. A wash in 1×TBS is performed for 3 minutes.

Slides are transferred to 1×TBS-T, then laid flat into staining tray. 3-4 drops of antibody diluent in dispensed onto the slide to cover the tissue section completely, and incubated for at least 10 minutes. The primary antibody is prepared, incubated, and washed. The secondary antibody-HRP is prepared, inbucated, and washed.

### Example 2: Generation of a Spectral Library

A spectral library with the OPAL 7-color fIHC Kit (PerkinElmer, NEL797001kt) is generated to establish baseline fluorescent information for the performance of quantitative image analysis.

Slides on appropriate FFPE tissue are determined by study plan. The slides include: auto-fluorescent slide, DAPI only slide, Opal 520 only slide, Opal 540 only slide, Opal 570 only slide, Opal 620 only slide, Opal 650 only slide, and Opal 690 only slide.

A single antibody stain on FFPE tissue is performed. A heat induced stringent wash is performed.

One the slides have been mounted, a fluorescence image is captured and stored.

### Example 3: Image Acquisition

Images of fluorescent IHC stained slides are captured to visualize immune cells in situ in formalin-fixed paraffin-embedded (FFPE) tissue sections.

The image capture device is manually focused onto the tissue. The entire slide is scanned. The field resolution is set to 20×. An image is captured manually.

### Example 4: Quantitative Image Analysis at the Cell-Segmented Level

Images from stained slides of in vivo and tissue samples are generated and analyzed. Phenotypes are quantifies, discriminated, and analyzed with multiple biomarkers in situ in FFPE.

PerkinElmer Vextra 3 inForm software, PerkinElmer OPAL 7-color fIHC kit, TSA reagent/antibodies, and FFPE control slides images are used.

FFPE control slides are as follows:

| | |
|---|---|
| Positive Control: Normal | Non-tumor antibodies |
| human tissue | |
| Positive Control: Tumor | Tumor-specific antibodies |
| human | |
| Negative Control (non-specific): | Host-specific isotype Antibody in place |
| Normal human tissue | of 1º Antibody, with 2º Antibody and |
| | TSA |
| Auto Fluorescent Control: | No 1º Antibodies, with all 2º Antibody |
| Sample Specific | and TSA non-tumor antibodies |

The fluorophores used in the study are selected. Cell segmentation settings are set. If the system erroneously counted cells, the parameters are adjusted. The image is phenotyped, with the filters adjusted accordingly to the phenotype and stain used. The cells that express the phenotype of interest are selected.

The nature of the native cancer interactome in a tumor microenvironment of tissues may be defined and quantified. For example, tissues of humans, murines, canines, bovines, filines, and equines may be defined and quantified. A method of multiplexed staining, followed by imaging analysis may be used to define and quantify the cancer interactome.

A number of benefits may be provided by quantifying the cancer interactome, such as: defining the relationship between distinct cell-types independently of cellular density, defining interaction partners and measuring changes in interaction partners based on clinical endpoints, defining cell-subpopulations that exist in their native context but remain unknown due to tumor analysis methods based on bulk-derived measurements or single cell assays, studying and determining relationships between phenotypically defined and phenotypically undetermined cell-types that are associated with a specific clinical endpoint (e.g., treatment outcome with anti-programmed cell death protein 1), and determining new insights based on cellular interactions that are not captured using other tumor analysis methods.

The following method may be used to define an interactome:

Step 1: Define Cell Boundaries and Label Matrix Vertices by Cell

Two inputs may be used to define an interactome. The first is a matrix representing the cellular image where boundaries between cells are defined. FIG. 1 provides a representative example matrix that defines cellular boundaries. The matrix represents a 9×9 image which was determined to be comprised of six cells. FIG. 2 provides a 9×9 representative example which corresponds with FIG. 1 and is an example of an intensity matrix, wherein the relative intensity of each marker of interest per pixel is defined.

Based on the boundaries defined in the cellular boundary input, each pixel is labeled based on the cellular boundary with which it is contained. Boundary pixels are labeled based on all cells sharing that boundary pixel. An example of pixel labeling is shown in FIG. 3. The matrix represents cell labeling from the boundary information from FIG. 1, wherein the symbol “I” indicates pixels that are boundary pixels and labeled by the cells that are part of that boundary.

Step 2: Measuring Concordance Between Single-Plex and Multiplex IHC Staining and Imaging

A key challenge in multiplexed IHC staining is determining the impact of multiplexing on the performance of any single antibody during testing. Single-plex and multiplex pixel intensity distributions are overlaid to confirm correspondence of staining intensity scales.

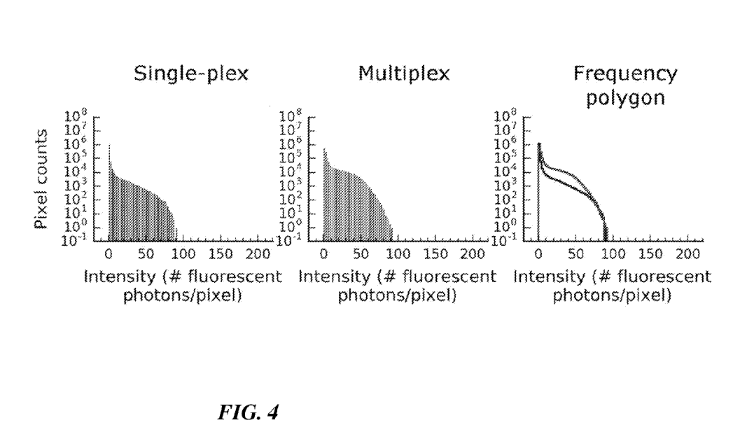

The following method is used to quantify the impact on intensity scales by comparing single-plex and multiplex staining thru frequency polygons. Frequency polygons of pixel staining intensities for example single-plex and multiplex images for a single channel are overlaid and visual inspection by study leadership confirms correspondence. An example is shown in FIG. 4. The distributions of positive pixels determined from step two for a single channel for both single-plex and multiplex are compared using the following method. Median and Median Absolute Deviation (MAD) are calculated for both distributions. The distance between medians is divided by the MAD of both distributions and the larger of the two resulting normalized distances is reported. A maximum normalized distance of less than 2.0 is labeled acceptable, between 2.0 and 4.0 is labeled marginal and above 4.0 is labeled not acceptable.

Step 3: Defining Positive and Negative Intensity Thresholds for Each Fluorophore

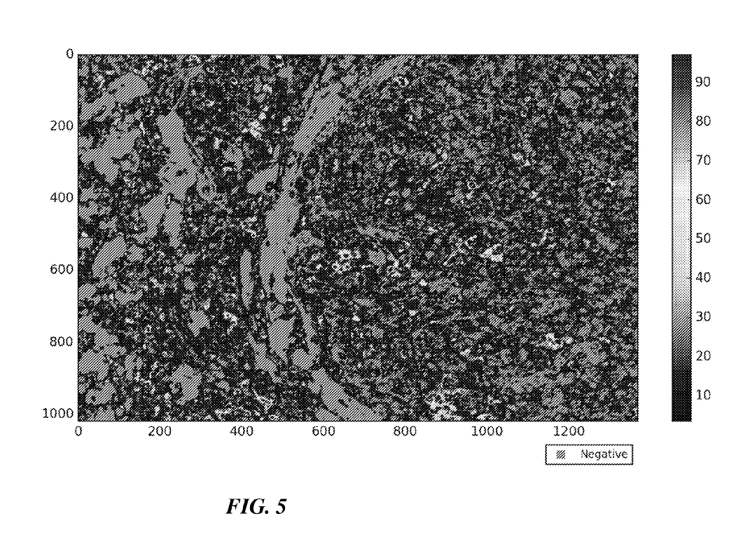

Raw data from fluorescence channels is provided using heatmap visualizations thresholded across the lower range of intensities. An appropriate threshold for positive staining is based on the marker of interest in a given study. FIG. 5 shows an example of positive intensity thresholding with heatmap visualization. A heatmap shows potential positive pixels for an image as intensities colored blue to red. Negative staining is shown in grey. Note that blue ranging 10-20 can also be defined as negative depending on positive and negative controls and the flourophore's range of dynamic signal.

Step 4: Calculate Total Readout Intensity for Each Cell and Each Readout

Intensities of pixels labeled as positive using the thresholds from step 2 within any cellular boundary (including boundary pixels) are summed over the cell area to provide the total marker intensity within the cell for each cell. This values are normalized for the total area of the cell.

Step 5: Label Cells as Positive or Negative for Each Readout

To accurately perform phenotyping, each the definition of positive/negative for a given marker is calibrated based on a reasonable sample of study data input from experimentalists. A set of calibration images is provided in which positive and negative can be performed manually or thru machine based learning. Phenotyping is calibrated by choosing an intensity distribution percentage cutoff where the distribution is calculated using the normalized intensity from step 4 for all cells with any positive intensity in each image. The algorithm is cross-validated within the calibration set and a minimum of 90% accuracy is required to use the calibration.

Step 7: Determine Adjacent Pixel Adjacency Graph

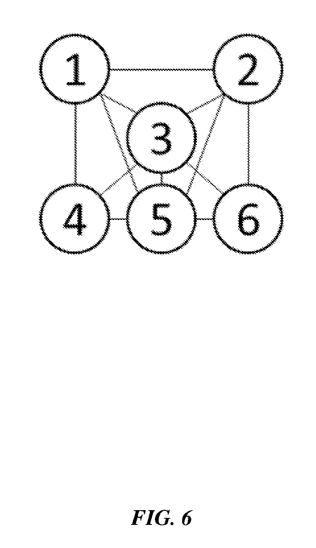

Image data (pixels and cells) can be converted into a mathematical graph that represents spatial adjacency relationships between the objects. Pixel adjacency may be defined as any two pixels that are within a distance of square-root(2) pixels, that is, direct adjacency or diagonal adjacency of any two pixels. A mathematical directed graph is constructed using this data where each node (vertex) represents a single pixel. Edges are added by iterating over all nodes and adding a single edge for each adjacent neighbor where the edge source is the node of interest and the edge target. In the case of pixels, the adjacency graph does not include pixels which are not positive for any marker of interest. An adjacency graph is shown in FIG. 6.

Step 8: Determine Cell Adjacency Graph

Cellular adjacency is defined as two cells where any two pixels with either cell (including boundary pixels) are within a distance of square-root(2) pixels. That is, direct adjacency or diagonal adjacency of any two pixels within the two cells is counted as cellular adjacency. An adjacency graph is created in a similar fashion to pixels where cells counted as adjacent are linked in a directed graph. Unlike the pixel data, cells which are not positive for any marker are included as “Other” cells.

Step 9: Calculate Fraction of Adjacency Between Pixels

Pixels are grouped according to intensity value positivity, where each group is a positive/negative set of one or more study readouts. Pixels can be part of more than one group. Pixel adjacency graphs are pooled across a given image set defined by the study objectives. For a given pair of pixel groups, the fraction of adjacency between a source group and target group is defined as (number edges with source as node in source group and target as node in target group)/(total number of edges with source as node in source group). Different image cohorts may contain different numbers of images and different numbers of pixels, but because fraction of adjacency is a normalized quantity, it is comparable regardless of these differences.

Fraction of adjacencies can be displayed when calculated all-by-all on a set of phenotype groups as heatmap. In these heatmaps, the source phenotype is presented on the rows and the target phenotype is presented on the columns. Because each row shares a source phenotype, all cells have within a row have an equal denominator. Each cell in the heatmap is calculated independently from each other cell.

Step 10: Calculate Fraction of Adjacency Between Cell-Types

Cells are grouped according to phenotypes of interest, where each phenotype is a positive/negative set of one or more study readouts. The adjacency heatmap is calculated using the same method as pixels.

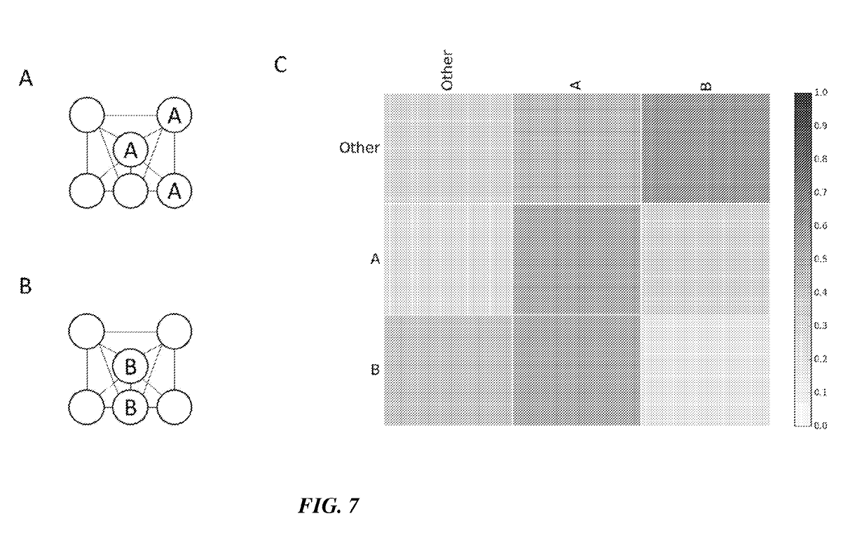

An example calculation and resulting heatmap are presented in FIG. 7 parts A-C. FIG. 7 part A gives an example cell adjacency network from FIG. 4 with cells phenotyped for readout “A”. FIG. 7 part B gives an example cell adjacency network from FIG. 4 with cells phenotyped with readout “B”.

FIG. 7 part C provides a calculated cell adjacency heatmap. It shows closer adjacency from A->A, B->A and from Other->B. Fractions of adjacency in a row may not add to 1 because of cells that are positive for multiple readouts.

Step 11: Calculate Differences in the Fraction of Adjacency Between Sample Groups.

Reference heat maps may be generated based on study objectives. In this instance, a reference heat map for non-responders and a reference heat map for responders may be generated.

If multiple image sets have been defined based on study objectives, fraction of adjacency differences can be calculated for each heatmap cell between two heatmaps of identical markers (either pixels or cells). The difference between fractions of adjacency for each source/target pair between a study reference image set and comparison set can be also reported as a heatmap. P-values for the differences can be calculated using Fisher's exact test by constructing a contingency table of the edge counts consisting of the two image sets.

FIG. 8 shows an adjacency heatmap of a subject that did not respond to treatment, or a non-responder subject. Fraction of adjacency heatmap were calculated on images taken from the invasive margin of subjects who were non-responsive to treatment. “Other” in this heatmap are cells which are not determined positive for any study readout. The heatmap shows high proximity between specific phenotypes such as CD20/CD20, CD19/CD20, and CD19/CD19.

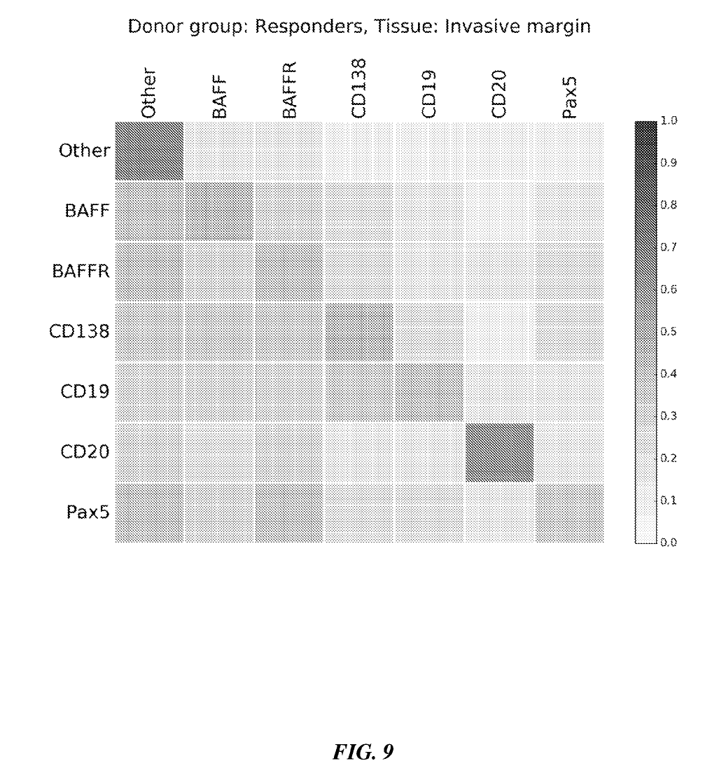

FIG. 9 shows a fraction of adjacency heatmap that was calculated on images taken from the invasive margin of subjects who were responsive to treatment. “Other” in this heatmap are cells which are not determined positive for any study readout.

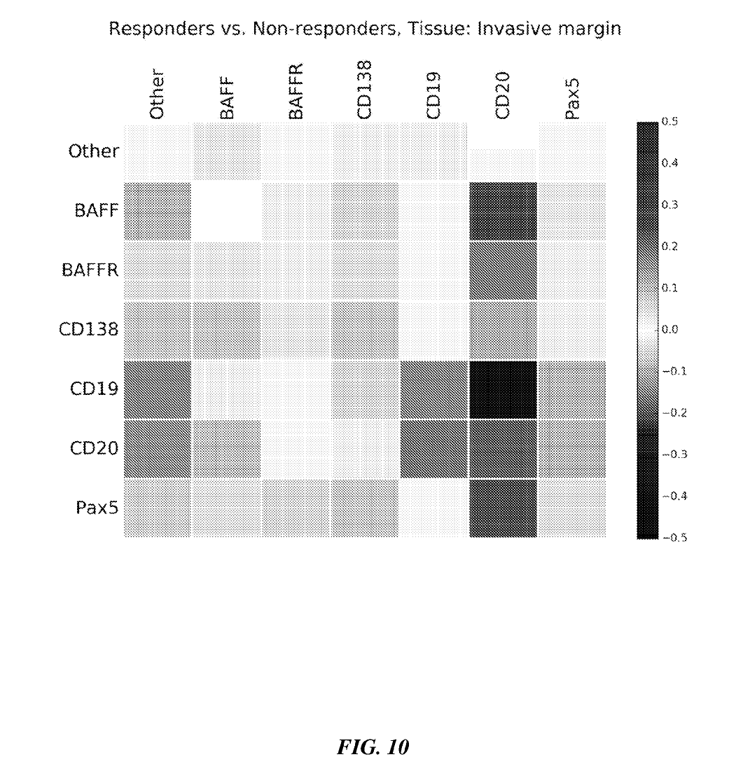

FIG. 10 shows an adjacency heatmap of the difference between the heatmap of a responder minus the heatmap of a non-responder. That is, the heatmap of FIG. 8 is subtracted from the heatmap of FIG. 9.

“Other” in this heatmap are cells which are not determined positive for any study readout. This heatmap shows lower proximity between CD20 and study phenotypes in responders as compared to non-responders and higher proximity between other cells and study phenotypes.

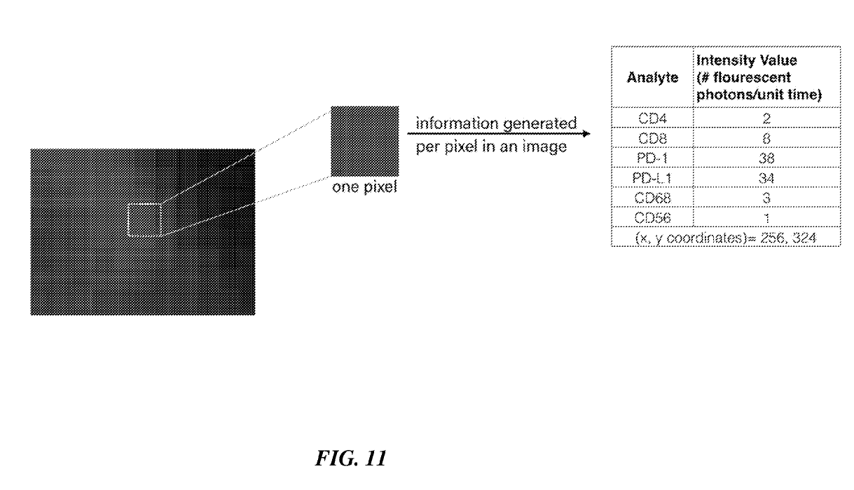

FIG. 11 illustrates the use of pixel-based analytics for diagnostic and therapeutic development. Each image of cells is comprised of approximately 1.5 million pixels where each pixel is defined in space according to x,y coordinate values and measures the relative intensity (e.g., relative expression level) of each analyte. FIG. 11 is a subsection of an image that was acquired on a tissue section tested for CD4, CD8, PD-1, PD-L1, CD68 and CD56 expression.

### Example 5: Antitumor Cell Efficacy of Compositions of the Disclosure

Cytotoxic activity of the compositions against human tumor cells are measured using an in vitro cell culture viability assay. Human A375 melanoma cells are incubated with 10 different composition concentrations between 10 nM and 100 μM for 72 hours and stained with a resazurin-based cell viability reagent to quantify viable cells. Living cells can reduce the non-fluorescent resazurin to a highly fluorescent resorufin, which can be quantified with a spectrophotometric plate reader. The fluorescence signal is normalized between the signals of untreated viable cells and the background signal of dead cells. Then, the half-maximum concentration for the inhibition of resazurin staining is calculated.

### Example 6: Cell Proliferation Assay

The ability of a composition of the present disclosure to inhibit the growth of cells, such as human leukemia, VCaP, LNCaP, 22RV1, DU145, LNCaP-AR, MV4;11, KOPN-8, ML-2, MOLM-13, bone marrow cells (BMCs), MLL-AF9, MLL-ENL, E2A-HLF, HL-60 and NB4 cells, is tested using a cell viability assay, such as the Promega CellTiter-Glo® Luminescent Cell Viability Assay (Promega Technical Bulletin, 2015, “CellTiter-Glo® Luminescent Cell Viability Assay”: 1-15, herein incorporated by reference in its entirety). Cells are plated at relevant concentrations, for example about 1×10⁵-2×10⁵ cells per well in a 96-well plate. A composition of the present disclosure is added at a concentration up to about 2 μM with eight, 2-fold serial dilutions for each composition. Cells are incubated at 37° C. for a period of time, for example, 72 hours, then cells in the control wells are counted. Media is changed to restore viable cell numbers to the original concentration, and compositions are re-supplied. Proliferation is measured about 72 hours later using Promega CellTiter-Glo® reagents, as per kit instructions.

### Example 7: Efficacy Study in Mouse Xenograft Tumor Model

Immunodeficient mice, such as 8-10 week-old female nude (nu/nu) mice, are used for in vivo efficacy studies in accordance with the guidelines approved by IACUC. Leukemia cells, such as human MV4-11 leukemia cells available from ATCC, are implanted subcutaneously via needle into female nude mice (5×10⁶ cells/mouse). When the tumor reaches a size of approximately 150 to 250 mm³ in mice, the tumor-bearing mice are randomly assigned to a vehicle control or composition treatment group (8 animals per group). Animals are treated with a composition of the present disclosure by oral gavage or intraperitoneal injection in an appropriate amount and frequency as can be determined by the skilled artisan without undue experimentation. Subcutaneous tumor volume in nude mice and mice body weight are measured twice weekly. Tumor volumes are calculated by measuring two perpendicular diameters with calipers (V=(length×width²)/2). Percentage tumor growth inhibition (% TGI=1−[change of tumor volume in treatment group/change of tumor volume in control group]*100) is used to evaluate anti-tumor efficacy. Statistical significance is evaluated using a one-tailed, two sample t test. P<0.05 is considered statistically significant.

### Example 8: Synergistic Efficacy Study in Mouse Xenograft Tumor Model

Immunodeficient mice, such as 8-10 week-old female nude (nu/nu) mice, are used for in vivo efficacy studies in accordance with the guidelines approved by IACUC. Leukemia cells, such as human MV4-11 leukemia cells available from ATCC, are implanted subcutaneously via needle into female nude mice (5×10⁶ cells/mouse). When the tumor reaches a size of approximately 150 to 250 mm³ in mice, the tumor-bearing mice are randomly assigned to one or four groups: a vehicle control, an anti-programmed cell death protein 1 monotherapy treatment group, a tabalumab treatment group, or a combination anti-programmed cell death protein 1/tabalumab treatment group (8 animals per group). All animals in the treatment groups are treated by oral gavage or intraperitoneal injection in an appropriate amount and frequency as can be determined by the skilled artisan without undue experimentation. Subcutaneous tumor volume in nude mice and mice body weight are measured twice weekly. Tumor volumes are calculated by measuring two perpendicular diameters with calipers (V=(length×width²)/2). Percentage tumor growth inhibition (% TGI=1−[change of tumor volume in treatment group/change of tumor volume in control group]*100) is used to evaluate anti-tumor efficacy. Statistical significance is evaluated using a one-tailed, two sample t test. P<0.05 is considered statistically significant. The change of tumor volume in the combination treatment group is larger than in the anti-programmed cell death protein 1 monotherapy treatment group, the tabalumab treatment group, and larger than the addition of the change of both groups separately.

### Example 9: Efficacy Study in Non-Small Cell Lung Cancer Xenograft Model

Immunodeficient mice, such as 4-6 week-old male CB17 severe combined immunodeficiency (SCID) mice, are used for in vivo efficacy studies in accordance with the guidelines approved by IACUC. Parental non-small cell lung cancer cells, such as VCaP or LNCaP-AR cells, are implanted subcutaneously into male CB.17.SCID mice (3-4×10⁶ cells in 50% Matrigel). When the tumor reaches a palpable size of approximately 80 mm³, the tumor-bearing mice are randomly assigned to a vehicle control or composition treatment group (6 or more animals per group). Animals are treated with a composition of the present disclosure by intraperitoneal injection in an appropriate amount and frequency as can be determined by the skilled artisan without undue experimentation. In one example, mice are treated with 40 mg/kg of a composition of the present disclosure daily by i.p. injection for two weeks, then 5 days per week thereafter. Subcutaneous tumor volume and mice body weight are measured twice weekly. Tumor volumes are calculated by measuring two perpendicular diameters with calipers (V=(length×width²)/2).

### Example 10: Efficacy Study in Castration-Resistant Non-Small Cell Lung Cancer Tumor Xenograft Model (VCaP)

Immunodeficient mice, such as 4-6 week-old male CB17 severe combined immunodeficiency (SCID) mice, are used for in vivo efficacy studies in accordance with the guidelines approved by IACUC. Parental non-small cell lung cancer cells, such as VCaP cells, are implanted subcutaneously into male CB.17.SCID mice (3-4×10⁶ cells in 50% Matrigel). When the tumor reaches a size of approximately 200-300 mm³, the tumor-hearing mice are physically castrated and tumors observed for regression and regrowth to approximately 150 mm³. The tumor-bearing mice are randomly assigned to a vehicle control or composition treatment group (6 or more animals per group). Animals are treated with a composition of the present disclosure by intraperitoneal injection in an appropriate amount and frequency as can be determined by the skilled artisan without undue experimentation. In one example, mice are treated with 40 mg/kg of a composition of the present disclosure daily by i.p. injection. Subcutaneous tumor volume and mice body weight are measured twice weekly. Tumor volumes are calculated by measuring two perpendicular diameters with calipers (V=(length×width²)/2).

### Example 11: Efficacy Study in Castration-Resistant Non-Small Cell Lung Cancer Xenograft Model (LNCaP-AR)

Immunodeficient mice, such as 4-6 week-old male CB17 severe combined immunodeficiency (SCID) mice, are used for in vivo efficacy studies in accordance with the guidelines approved by IACUC. CB.17.SCID mice are surgically castrated and allowed to recover for 2-3 weeks before implanting parental non-small cell lung cancer cells, such as LNCaP-AR cells, subcutaneously into (3-4×10⁶ cells in 50% Matrigel). When the tumor reaches a size of approximately 80-100 mm³, the tumor-bearing mice are randomly assigned to a vehicle control or composition treatment group (6 or more animals per group). Animals are treated with a composition of the present disclosure by intraperitoneal injection in an appropriate amount and frequency as can be determined by the skilled artisan without undue experimentation. In one example, mice are treated with 60 mg/kg of a composition of the present disclosure daily by i.p. injection for 27 days. Subcutaneous tumor volume and mice body weight are measured twice weekly. Tumor volumes are calculated by measuring two perpendicular diameters with calipers (V=(length×width²)/2).

### Example 12: Conservation of Type II Signature

Tumor samples were obtained from subjects with squamous non-small cell lung cancer, adeno non-small cell lung cancer, colorectal cancer, head and neck squamous cell carcinoma, breast cancer, or melanoma.

Tumor samples were stained and imaged, and determined to possess a Type II Resistant Signature.

### Example 13: Type II Signature

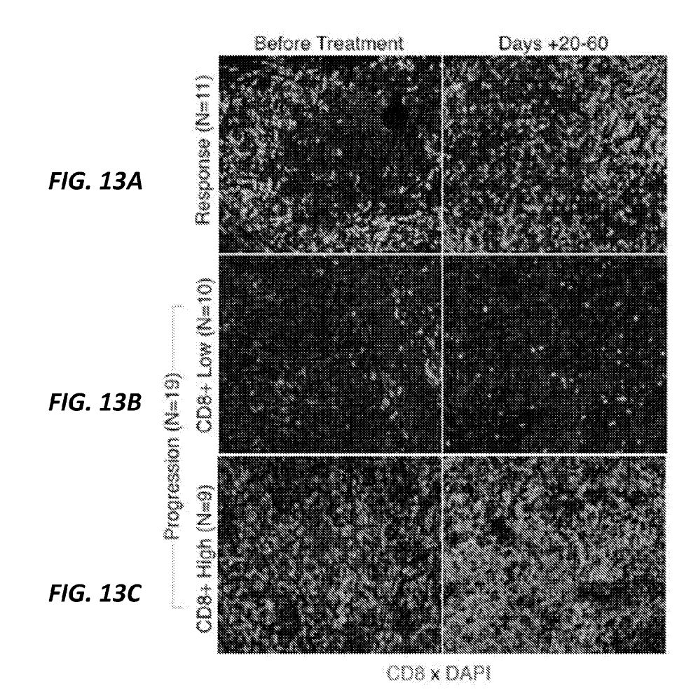

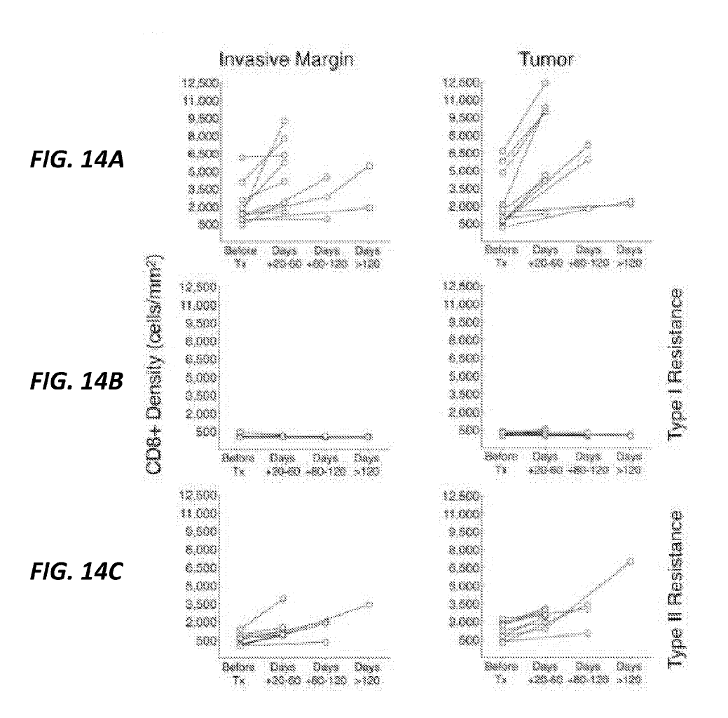

FIGS. 13A, 13B, and 13C, along with FIGS. 14A, 14B, and 14C, illustrate three types of tumors before and after treatment. FIG. 13A illustrates a traditionally “hot” tumor, with a high level of CD8 cells. This “hot” tumor responds to single agent treatment. Before treatment, the “hot” tumor is expected to respond to treatment. As illustrated in FIG. 14A, the amount of CD8 density increase during treatment at both the invasive margin and within the tumor itself.

FIG. 13B illustrates a traditionally “cold” tumor, one that is not expected to, and does not, respond to single agent treatment. The CD8 density at both the invasive margin and within the tumor (FIG. 14B) stays consistent and does not increase.

FIG. 13C illustrates a tumor that would be traditionally labeled as a “hot” tumor because of the high density of CD8 cells, and would be expected to respond to single agent therapy. However, this tumor does not respond to single agent therapy during treatment. The level of CD8 density does increase, as illustrated in FIG. 14C, and mimics a responder (FIG. 14A) pattern. Thus, FIG. 13C and FIG. 14C illustrate a Type II Signature or Type II Resistant tumor.

### Example 14: Proliferation of Type I Resistant Cells

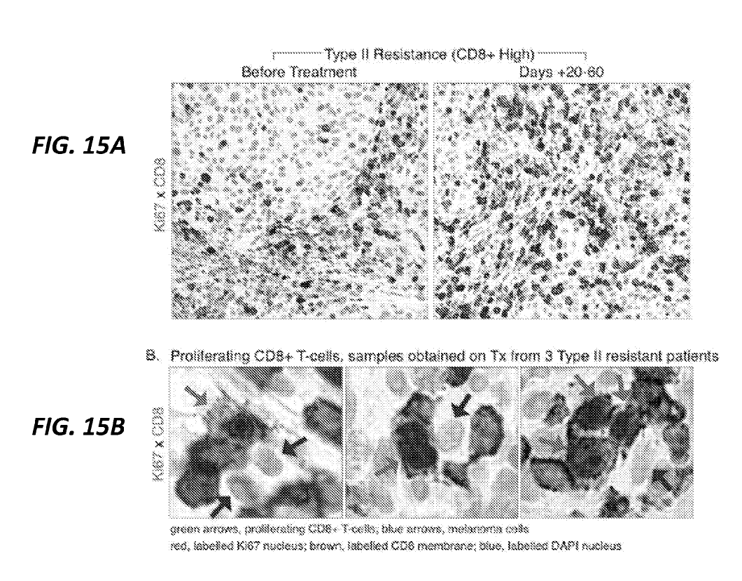

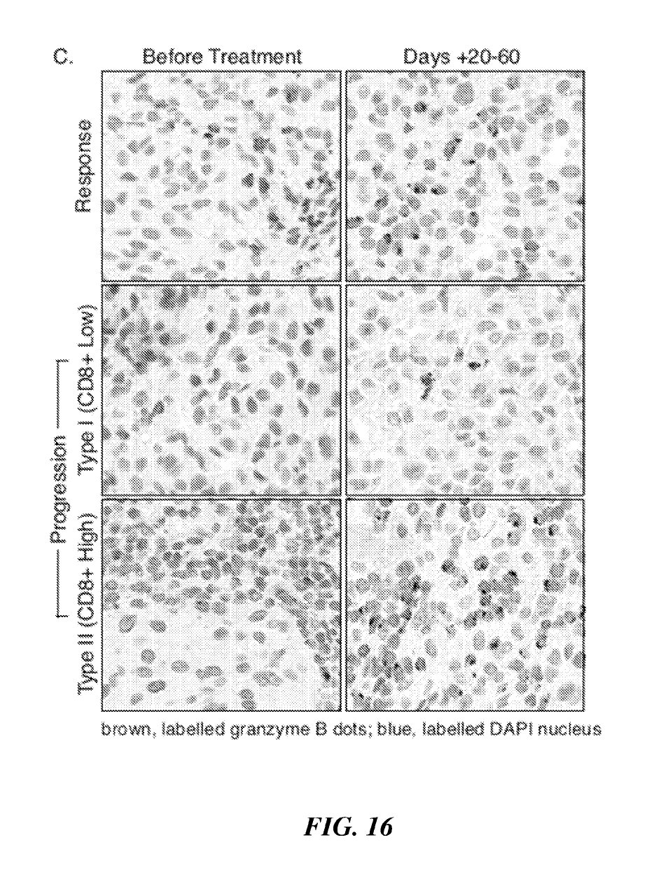

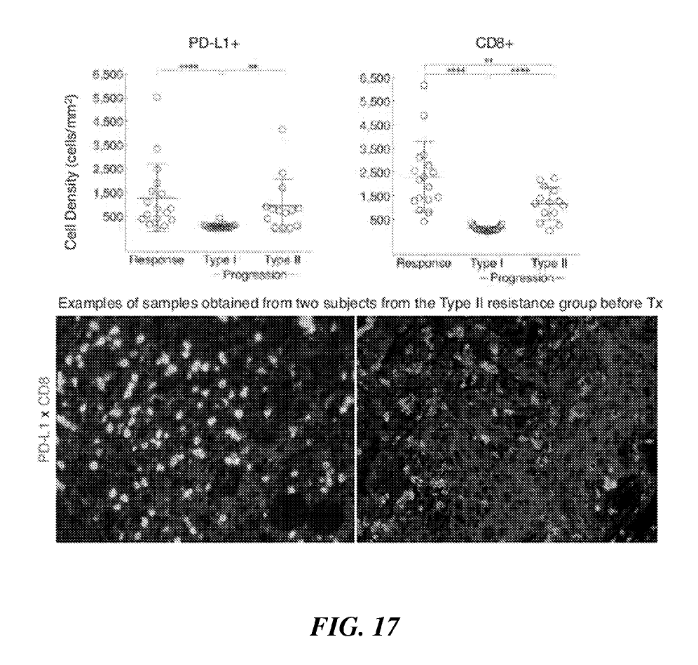

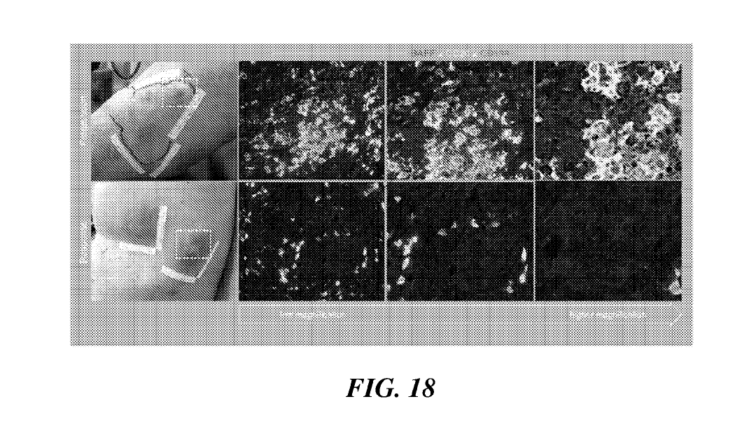

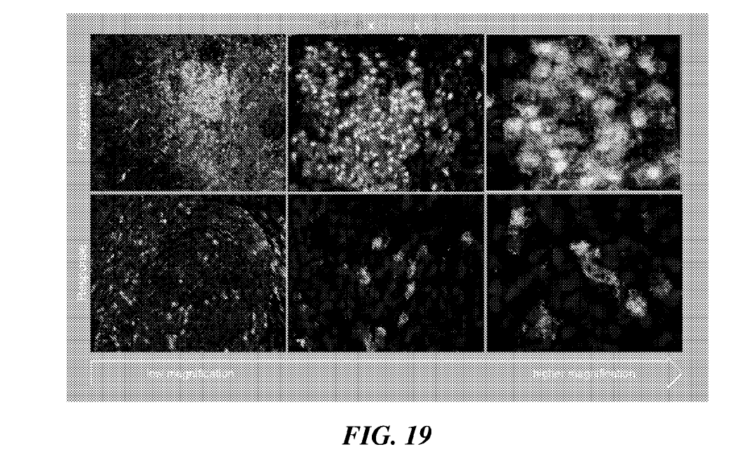

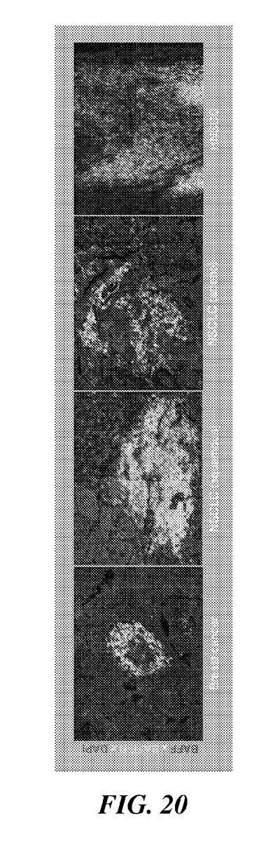

FIG. 15A and FIG. 15B illustrate Type II resistant tumors and the evidence of cell proliferation in these tumors before and during anti-PD1 treatment. FIG. 15B provides evidence for proliferating CD8 T-cells with samples obtained during treatment from three Type 11 resistant subjects. The T-cells are proliferating as well as turned on by secreting granzyme B.

### Example 15: Dewaxing and Antigen Retrieval

Dilute concentrated Hot Rinse at a ratio of 1:25. Fill staining jar (100 mL) or metal slide canister with (250 mL) of 1× Hot Rinse. Fill a second container with 100-250 mL of 1X Borg Decloaker RTU. Fill Decloaking Chamber with 500 mL Deionized Water. Heat to 110° C. for 15 minutes. When the solution reaches 80-125° C., the solution turns a faint purple color and indicates that the high temperature solution is at correct pH. A yellow or red solution indicates an incorrect pH. Transfer slides to the slide container with 1× Hot Rinse and dunk vigorously for a minimum of 20 times. Gently rinse by gradually adding Deionized Water to the solution. Use a total of 500 mL of Deionized Water in this step Pour off ¼ of Hot Rinse out of staining jar. Top off with Deionized Water, dunk vigorously for a minimum of 20 times. Repeat until all of the of Deionized Water is used. Hold slides in fresh Deionized Water until next step. Turn off decloaking chamber.

### Example 16: Endogenous Peroxidase Blocking

Prepare 250 ml of 0.3% Hydrogen Peroxide in a staining jar. Add 2.5 ml 30% Hydrogen Peroxide in 247.5 ml of Deionized Water. Transfer slides to 0.3% Hydrogen Peroxide, and dunk a minimum of 20 times. Incubate for 10 minutes+/−1 minute. Transfer slides to 1×TBS. Dunk vigorously for a minimum of 20 times to wash. Hold slides in 1×TBS until Fluorescent IHC Staining. Slides may be stored overnight at 2-8° C. in a staining jar containing fresh 1×TBS.

### Example 17: Antibody Incubation

Place slides into 1×TBS-T, and agitate at least 5 dips prior to staining. Incubate for at least 30 minutes no more than 1 hour. Remove excess Antibody by aspiration. Wash with fresh 1×TBS-T, and dunk vigorously. Allow slides to equilibrate for at least 3 min. Dunk again. Transfer to a new TBS-T wash, and repeat the dunk-equilibrate-dunk for a total of 4 washes. Hold slides in last wash until Secondary Antibody HRP Incubation.

### Example 18: Secondary Antibody-HRP Incubation

Apply 200 μL of Opal Polymer HRP Ms+Rb (Perkin Elmer). Incubate for at least 10 minutes and no more than 12 minutes. Remove excess Secondary Antibody by aspiration. Wash slides with 1×TBS-T prior to placing into slide holder, or place slides into slide holder and dunk at least 5 dips to pre-wash with 1×TBS-T. To wash, transfer slides to fresh 1×TBS-T, and dunk vigorously for a minimum of 20 times. Allow slides to equilibrate for at least 3 min. Dunk again for a minimum of 20 dips. Transfer to a new TBS-T wash, and repeat the dunk-equilibrate-dunk for a total of 3 washes. Hold slides in last wash until TSA incubation.

### Example 19: TSA Incubation

Apply 150 μL/slide of TSA Reagent. Incubate for at least 10 minutes and no more than 11 minutes. Remove excess TSA Reagent by aspiration. Irrigate slides with 1×TBS-T prior to placing into slide holder, or place slides into slide holder and dunk at least 5 dips to pre-wash with 1×TBS-T. To wash, transfer slides to fresh 1×TBS-T, and dunk vigorously for a minimum of 20 times. Allow slides to equilibrate for at least 3 min. Dunk again for a minimum of 20 dips. Transfer to a new TBS-T wash, and repeat the dunk-equilibrate-dunk for a total of 3 washes. Hold slides in last wash until Heat Induced Stringent Wash.

### Example 20: Heat Induced Stringent Wash

Prepare 250 mL of 1×AR6 Buffer from the 7-color Fluorescent IHC Kit (Perkin Elmer, NEL797001) using Deionized Water. Place the covered staining jar with slides into a 1000-Watt microwave oven, and place a second jar containing 250 mL Deionized Water to balance the heat distribution during the cycle. Heat in microwave. Gently cool by gradually adding 1×TBS to the jar. Pour off ¼ of Citrate buffer out of staining jar. Top off with 1×TBS and agitate a minimum of 10 dips. Repeat until 500 mL of TBS is used. Transfer slides into fresh 1×TBS wash, dip the slide rack up and down at least 5 times. Hold slides in 1×TBS before proceeding with next step. If multiplexing more antibodies proceed to section 15, Sequential IHC Staining for Multiplex.

### Example 21: Nuclear Counterstain

Prepare DAPI solution from the TSA kit at concentration of 2 drops/1 mL TBS-T. DAPI Counterstain. Transfer slides to staining jar filled with 1×TBS-T, and dunk at least 5 times. Apply 200 μL DAPI solution, and incubate in the staining tray for 5 minutes+/−1 minute. Transfer to a new wash of 1×TBS-T. Dunk a minimum of 20 times, and allow slides to equilibrate for 5 min. Dunk slides a minimum of 20 times. Transfer to a new wash of 1×TBS. Dunk a minimum of 20 times, and allow slides to equilibrate for 5 min. Dunk slides a minimum of 20 times. Transfer to a new wash of deionized water, and dunk a minimum of 20 times.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein may be employed in practicing the invention. It is intended that the following claims define the scope of the invention and that methods and structures within the scope of these claims and their equivalents be covered thereby.

1.-74. (canceled)

75. A method of treating a subject having a cancer or a tumor comprising:
a. administering to said subject, a first agent comprising a therapeutically effective amount of a B-cell activating factor (BAFF) inhibitor or a salt thereof, or a B-cell activating factor receptor (BAFF-R) inhibitor or a salt thereof; and
b. administering to said subject, a second agent comprising a therapeutically effective amount of a checkpoint inhibitor or a salt thereof.

76. The method of claim 75, wherein said first agent and said second agent are administered sequentially or concurrently.

77. The method of claim 75, wherein said subject was previously treated with a first checkpoint inhibitor or a salt thereof.

78. The method of claim 77, wherein said cancer or said tumor was at least partially refractive to said first checkpoint inhibitor or a salt thereof.

79. The method of claim 75, wherein said first agent comprises said BAFF inhibitor or said salt thereof.

80. The method of claim 79, wherein said BAFF inhibitor or said salt thereof is tabalumab or a salt thereof, belimumab or a salt thereof, or a combination thereof.

81. The method of claim 75, wherein said first agent comprises said BAFF-R inhibitor or said salt thereof.

82. The method of claim 75, wherein said checkpoint inhibitor or said salt thereof is a PD-1 inhibitor or a salt thereof, PD-L1 inhibitor or a salt thereof, PD-L2 inhibitor or a salt thereof.

83. The method of claim 82, wherein said checkpoint inhibitor or said salt thereof is said PD-1 inhibitor or said salt thereof.

84. The method of claim 83, wherein said PD-1 inhibitor or said salt thereof is nivolumab or a salt thereof, pembrolizumab or a salt thereof, pidilizumab or a salt thereof, or a combination thereof.

85. The method of claim 82, wherein said checkpoint inhibitor is a PD-L1 inhibitor or a salt thereof.

86. The method of claim 85, wherein said PD-L1 inhibitor or said salt thereof is atezolizumab or a salt thereof, avelumab or a salt thereof, durvalumab or a salt thereof, or a combination thereof.

87. The method of claim 75, wherein said cancer or said tumor is a Type II resistant tumor or cancer.

88. A method of selecting a therapeutic regimen, comprising:
a. determining a presence or absence of cluster of differentiation 8 (CD8, and at least one of: B-cell activating factor (BAFF), B-cell activating factor receptor (BAFF-R), or paired box Pax-5 (PAX5) in a cancer or a tumor of a subject;
b. detecting a presence of CD8 and at least one of BAFF, BAFF-R, or PAX5 in said cancer or said tumor and selecting a therapeutic regimen comprising a checkpoint inhibitor or a salt thereof and an immunotherapeutic agent or a salt thereof; or
c. detecting an absence of CD8 and at least one of BAFF, BAFF-R, or PAX5 in said cancer or said tumor and selecting a therapeutic comprising said checkpoint inhibitor or said salt thereof.

89. The method of claim 88, wherein said immunotherapeutic agent or said salt thereof inhibits a B-cell activating factor receptor cascade, a transmembrane activator and calcium-modulator and cyclophilin ligand interactor receptor cascade, a B-cell maturation antigen receptor cascade, or any combination thereof.

90. The method of claim 89, wherein said immunotherapeutic agent or said salt thereof is a B-cell activating factor (BAFF) inhibitor or a salt thereof, a B-cell activating factor receptor (BAFF-R) inhibitor or a salt thereof, or a combination thereof.

91. The method of claim 88, wherein said checkpoint inhibitor or said salt thereof is a PD-1 inhibitor or a salt thereof, PD-L1 inhibitor or a salt thereof, PD-L2 inhibitor or a salt thereof, or a combination thereof.

92. The method of claim 88, further comprising administering said therapeutic regiment or said therapeutic to said subject based on (b) or (c).

93. The method of claim 92, wherein said therapeutic regiment or said therapeutic maintains at least partial remission of said cancer or said tumor.
